(19) United States
(12) Patent Application Publication
(10) Pub. No.: US 2020/0254002 A1
(43) Pub. Date: Aug. 13, 2020

(54) COMBINATION THERAPIES FOR TREATING MUSCULAR DYSTROPHY

(71) Applicants: SAREPTA THERAPEUTICS, INC., Cambridge, MA (US); CATABASIS PHARMACEUTICALS, INC., Cambridge, MA (US)

(72) Inventors: Marco A. PASSINI, Cambridge, MA (US); Jill C. MILNE, Cambridge, MA (US); Andrew J. NICHOLS, Cambridge, MA (US)

(73) Assignees: SAREPTA THERAPEUTICS, INC., Cambridge, MA (US); CATABASIS PHARMACEUTICALS, INC., Cambridge, MA (US)

(21) Appl. No.: 16/649,320

(22) PCT Filed: Sep. 28, 2018
(86) PCT No.: PCT/US2018/053543

§ 371 Date: Mar. 20, 2020

Related U.S. Application Data
(60) Provisional application No. 62/565,016, filed on Sep. 28, 2017.
(60) Provisional application No. 62/737,750, filed on Sep. 27, 2018.

(51) Int. Cl. A61K 31/7125; A61P 21/00

(52) U.S. Cl. CPC A61K 31/7125; A61K 45/06; A61P 21/00

(57) ABSTRACT

The present disclosure relates to methods of treating Duchenne's Muscular Dystrophy by administering an antisense oligonucleotide that induces exon skipping and a non-steroidal anti-inflammatory compound.

### CROSS-REFENCE TO RELATED APPLICATIONS

This application claims priority to U.S. Provisional Application No. 62/565,016, filed Sep. 28, 2017 and U.S. Provisional Application No. 62/737,750, filed Sep. 27, 2018; which applications are each incorporated herein by reference in their entireties.

### FIELD

This disclosure relates to the field of muscular dystrophy, in particular, methods for treating Duchenne muscular dystrophy (DMD) and inducing the production of the protein, dystrophin, the lack of which is associated with the clinical manifestations of DMD.

### BACKGROUND OF THE DISCLOSURE

Duchenne Muscular Dystrophy (DMD) is a serious, progressively debilitating, and ultimately fatal inherited X-linked neuromuscular disease. DMD is caused by mutations in the dystrophin gene characterized by the absence, or near absence, of functional dystrophin protein that disrupt the mRNA reading frame, resulting in a lack of dystrophin, a critically important part of the protein complex that connects the cytoskeletal actin of a muscle fiber to the extracellular matrix. In the absence of dystrophin, patients with DMD follow a predictable disease course. Affected patients, typically boys, develop muscle weakness in the first few years of life, lose the ability to walk during childhood, and usually require respiratory support by their late teens. Loss of functional abilities leads to loss of independence and increasing caregiver burden. Once lost, these abilities cannot be recovered. Despite improvements in the standard of care, such as the use of glucocorticoids, DMD remains an ultimately fatal disease, with patients usually dying of respiratory or cardiac failure in their mid to late 20s.

Progressive loss of muscle tissue and function in DMD is caused by the absence or near absence of functional dystrophin; a protein that plays a vital role in the structure and function of muscle cells. A potential therapeutic approach to the treatment of DMD is suggested by Becker muscular dystrophy (BMD), a milder dystrophinopathy. Both dystrophinopathies are caused by mutations in the DMD gene. In DMD, mutations that disrupt the pre-mRNA reading frame, referred to as “out-of-frame” mutations, prevent the production of functional dystrophin. In BMD, “in-frame” mutations do not disrupt the reading frame and result in the production of internally shortened, functional dystrophin protein.

An important approach for restoring these “out-of-frame” mutations is to utilize an antisense oligonucleotide to exclude or skip the molecular mutation of the DMD gene (dystrophin gene). The DMD or dystrophin gene is one of the largest genes in the human body and consists of 79 exons. Antisense oligonucleotides (AONs) have been specifically designed to target specific regions of the pre-mRNA, typically exons to induce the skipping of a mutation of the DMD gene thereby restoring these out-of-frame mutations in-frame to enable the production of internally shortened, yet functional dystrophin protein.

The skipping of exon 53 in the dystrophin gene has been an area of interest for certain research groups due to it being the most prevalent set of mutations in this disease area, representing 8% of all DMD mutations. A prominent AON being developed by Sarepta Therapeutics, Inc., for DMD patients that are amenable to exon 53 skipping is golodirsen. Golodirsen is a phosphorodiamidate morpholino oligomer, or PMO. Another AON being developed by Nippon Shinyaku CO., LTD., for DMD patients that are amenable to exon 53 skipping is viltolarsen (NS-065 which is a PMO.

Exondys 51® (eteplirsen), is another PMO that was approved in 2016 by the United States Food and Drug Administration (FDA) for the treatment of Duchenne muscular dystrophy (DMD) in patients who have a confirmed mutation of the DMD gene that is amenable to exon 51 skipping. However, the current standard of care guidelines for the treatment of DMD in patients that are not amenable to exon 51 skipping include the administration of glucocorticoids in conjunction with palliative interventions. While glucocorticoids may delay the loss of ambulation, they do not sufficiently ameliorate symptoms, modify the underlying genetic defect or address the absence of functional dystrophin characteristic of DMD.

Previous studies have tested the efficacy of an antisense oligonucleotides (AON) for exon skipping to generate at least partially functional dystrophin in combination with a steroid for reducing inflammation in a DMD patient (see WO 2009/054725 and van Deutekom, et al., N. Engl. J. Med. 2007; 357:2677-86, the contents of which are hereby incorporated herein by reference for all purposes). However, treatment with steroids can result in serious complications, including compromise of the immune system, reduction in bone strength, and growth suppression. Notably, none of the previous studies suggest administering an antisense oligonucleotide for exon skipping with a non-steroidal anti-inflammatory compound to a patient for the treatment of DMD.

Thus, there remains a need for improved methods for treating muscular dystrophy, such as DMD and BMD in patients.

### SUMMARY OF THE DISCLOSURE

It is recognized that the absence of functional dystrophin in DMD patients causes muscle fibers to be more vulnerable to mechanical stress, and results in the activation of the NF-kB pathway. This leads to muscle inflammation, muscle damage and the reduced ability of muscles to regenerate. Nuclear factor κB (NF-κB) is an evolutionarily conserved, polymorphic, and pleiotropic system of transcriptional regulation designed to respond to cellular stress in a rapid and transient manner, promoting cell survival. Canonical NF-κB (cNF-κB) signaling involves activation of p65-p50 heterodimers by IKK-mediated release from IκB. During this process, IκB is phosphorylated by the IKK complex and is rapidly degraded by the proteasome to release the p65-p50 heterodimer, allowing nuclear translocation and subsequent transcriptional activation of NF-κB-responsive genes. Typical cNF-κB-induced genes include inflammatory cytokines and cNF-κB feedback regulatory products to counter p65-dependent activity. An IκB-independent, alternative NF-κB pathway (altNF-κB) exists that involves the activation of RelB-p52 heterodimers by IKKα-induced proteolytic cleavage of p100 into p52. Additionally, phosphorylation of a pool of IκB-independent p65 on Ser536 has been reported to result in p65-p65 homodimer formation and activation of genes distinct from cNF-κB activation; however, recent evidence suggests this modification serves as a brake on p65-dependent transcription.

Though these pathways are essential to organism survival and adaptation, chronic activation of the NF-κB system results in uncontrolled inflammatory pathology. Such is the case in dystrophin-deficient muscle, where chronic activation of cNF-κB occurs in the muscle of dystrophic mice and DMD patients. In agreement with NF-κB-dependent pathogenesis, genetic haploinsufficiency experiments in the mdx mouse model of DMD have confirmed that reduction of p65, but not p50, improves the dystrophic phenotype and affects both the muscle fibers and immune infiltrate. Accordingly, inhibition of NF-κB in dystrophic muscle via gene therapy with a dominant-negative IKKα or IKKβ or peptide-based IKKγ inhibitors has impressive therapeutic potential; however, both of these strategies are problematic for immediate translation.

An inhibitor of NF-kB of particular interest is edasalonexent, also known as CAT-1004. Edasalonexent is a small molecule conjugate of salicylate and docosahexaenoic acid (DHA) in development to treat inflammation associated with DMD by modulating the NF-kB pathway. A clinical trial (NCT02439216) is underway to determine if edasalonexent has beneficial effects in DMD patients with a determination of muscle composition and inflammation as measured by MRI being a primary outcome measure. Edasalonexent was shown to be safe, well tolerated, and inhibited activated NF-kB pathways in a phase I clinical program in adults (see Donovan et al., The Journal of Clinical Pharmacology, 2017, 57(5), 627-637, incorporated herein by reference). Another inhibitor of NF-kB of particular interest is CAT-1041, a conjugate of salicylate and EPA. CAT-1041 is a surrogate and analog of CAT-1004.

In one aspect, the present disclosure is directed to a method for treating Duchenne muscular dystrophy (DMD) in a patient in need thereof having a mutation of the DMD gene that is amenable to skipping exon 53, comprising administering to the patient an effective amount of golodirsen and an effective amount of a non-steroidal anti-inflammatory compound, thereby treating the patient with DMD.

In one aspect, the present disclosure is directed to a method for treating Duchenne muscular dystrophy (DMD) in a patient in need thereof having a mutation of the DMD gene that is amenable to skipping exon 53, comprising administering to the patient an effective amount of viltolarsen and an effective amount of a non-steroidal anti-inflammatory compound, thereby treating the patient with DMD.

In another aspect, the present disclosure provides a method for inducing or increasing dystrophin protein production in a patient with Duchenne muscular dystrophy (DMD) in need thereof who has a mutation of the DMD gene that is amenable to skipping exon 53, comprising administering to the patient an effective amount of golodirsen and an effective amount of a non-steroidal anti-inflammatory compound, thereby inducing or increasing dystrophin protein production in the patient. In another aspect, the present disclosure provides a method for inducing or increasing dystrophin protein production in a patient with Duchenne muscular dystrophy (DMD) in need thereof who has a mutation of the DMD gene that is amenable to skipping exon 53, comprising administering to the patient an effective amount of viltolarsen and an effective amount of a non-steroidal anti-inflammatory compound, thereby inducing or increasing dystrophin protein production in the patient.

In one aspect, the present disclosure is directed to a method for treating Duchenne muscular dystrophy (DMD) in a patient in need thereof having a mutation of the DMD gene that is amenable to skipping exon 53, comprising administering to the patient an effective amount of an antisense oligomer conjugate of the Formula

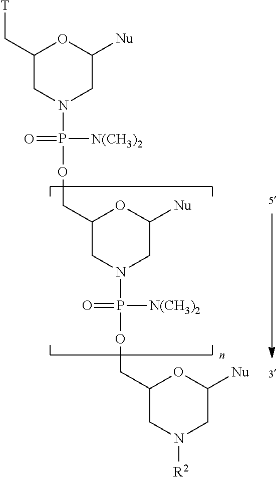

or a pharmaceutically acceptable salt thereof, wherein:

each Nu is a nucleobase which taken together form a targeting sequence; and

T is a moiety selected from:

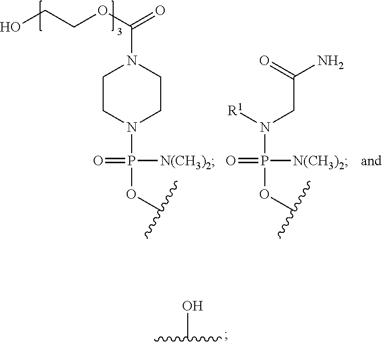

and

R¹ is Cₗ-C₆ alkyl,

R² is selected from H, acetyl or a cell penetrating peptide comprising a sequence selected from one of SEQ ID NO:11-19 and

n is from 16 to 28;

wherein the targeting sequence is selected from one of SEQ ID NO:1-10 and 20; and an effective amount of a non-steroidal anti-inflammatory compound, thereby treating the patient with DMD. In one aspect, R² is a cell penetrating peptide consisting of SEQ ID NO: 19. In one aspect, n is 23 and the targeting sequence is SEQ ID NO:1.

In another aspect, the present disclosure provides a method for inducing or increasing dystrophin protein production in a patient with Duchenne muscular dystrophy (DMD) in need thereof who has a mutation of the DMD gene that is amenable to skipping exon 53, comprising administering to the patient an effective amount of an antisense oligomer conjugate of the Formula

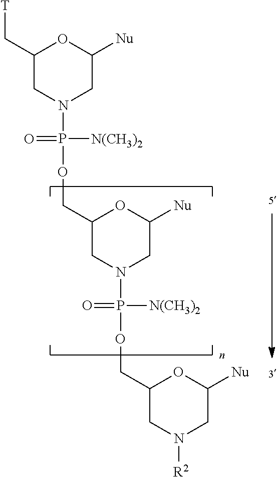

or a pharmaceutically acceptable salt thereof, wherein:

each Nu is a nucleobase which taken together form a targeting sequence; and

T is a moiety selected from:

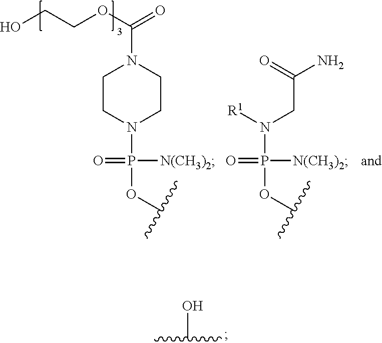

and

R¹ is Cₗ-C₆ alkyl,

R² is selected from H, acetyl or a cell penetrating peptide comprising a sequence selected from one of SEQ ID NO:11-19 and

n is from 16 to 28;

wherein the targeting sequence is selected from one of SEQ ID NO:1-10 and 20; and an effective amount of a non-steroidal anti-inflammatory compound, thereby treating the patient with DMD. In one aspect, R² is a cell penetrating peptide consisting of SEQ ID NO: 19. In one aspect, n is 23the targeting sequence is SEQ ID NO:1.

In some embodiments, the non-steroidal anti-inflammatory compound is an NF-kB inhibitor. For example, in some embodiments, the NF-kB inhibitor is edasalonexent, also referred to herein as CAT-1004, or a pharmaceutically acceptable salt thereof. In various embodiments, the NF-kB inhibitor may be a conjugate of salicylate and DHA. In some embodiments, the NF-kB inhibitor is CAT-1041 or a pharmaceutically acceptable salt thereof. In certain embodiments, the NF-kB inhibitor is a conjugate of salicylate and EPA. In various embodiments, the NF-kB inhibitor is

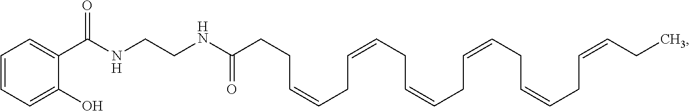 [SMILES: CC/C=C\C/C=C\C/C=C\C/C=C\C/C=C\C/C=C\CCC(=O)NCCNC(=O)c1ccccc1O]

or a pharmaceutically acceptable salt thereof.

In some embodiments, golodirsen is administered at a dose of 30 mg/kg weekly.

In some embodiments, viltolarsen is administered at a dose of 40 mg/kg. In some embodiments, viltolarsen is administered at a dose of 80 mg/kg.

In some embodiments, the antisense oligomer is administered at a dose of 30 mg/kg weekly. In some embodiments, the antisense oligomer is administered at a dose of 10 mg/kg weekly. In some embodiments, the antisense oligomer is administered at a dose of 20 mg/kg weekly.

In some embodiments, the antisense oligomer, such as golodirsen, is administered weekly for at least 12 weeks.

In various embodiments, CAT-1004 is administered at a dose of 33 mg/kg/day, 67 mg/kg/day, or 100 mg/kg/day.

In certain embodiments, the non-steroidal anti-inflammatory compound is administered for at least 12 weeks.

In various embodiments, the non-steroidal anti-inflammatory compound is administered prior to, in conjunction with, or subsequent to administration of the antisense oligomer, such as golodirsen. In some embodiments, the antisense oligomer and the non-steroidal anti-inflammatory compound are administered simultaneously. In some embodiments, the antisense oligomer and the non-steroidal anti-inflammatory compound are administered sequentially. In certain embodiments, the antisense oligomer is administered prior to administration of the non-steroidal anti-inflammatory compound. In various embodiments, the non-steroidal anti-inflammatory compound is administered prior to administration of the antisense oligomer.

In some embodiments, the antisense oligomer, such as golodirsen, is administered intravenously. In some embodiments, the antisense oligomer is administered as an intravenous infusion over 35 to 60 minutes.

In some embodiments, the non-steroidal anti-inflammatory compound is administered orally.

In various embodiments, the patient is seven years of age or older. In certain embodiments, the patient is between about 6 months and about 4 years of age. In some embodiments, the patient is between about 4 years of age and 7 years of age.

In some embodiments, combination treatment with the antisense oligomer, such as golodirsen, and a non-steroidal anti-inflammatory compound induces or increases novel dystrophin production, delays disease progression, slows or reduces the loss of ambulation, reduces muscle inflammation, reduces muscle damage, improves muscle function, reduces loss of pulmonary function, and/or enhances muscle regeneration, and any combination thereof. In some embodiments, treatment maintains, delays, or slows disease progression. In some embodiments, treatment maintains ambulation or reduces the loss of ambulation. In some embodiments, treatment maintains pulmonary function or reduces loss of pulmonary function. In some embodiments, treatment maintains or increases a stable walking distance in a patient, as measured by, for example, the 6 Minute Walk Test (6MWT). In some embodiments, treatment maintains, improves, or reduces the time to walk/run 10 meters (i.e., the 10 meter walk/run test). In some embodiments, treatment maintains, improves, or reduces the time to stand from supine (i.e, time to stand test). In some embodiments, treatment maintains, improves, or reduces the time to climb four standard stairs (i.e., the four-stair climb test). In some embodiments, treatment maintains, improves, or reduces muscle inflammation in the patient, as measured by, for example, MRI (e.g., MRI of the leg muscles). In some embodiments, MRI measures a change in the lower leg muscles. In some embodiments, MRI measures T2 and/or fat fraction to identify muscle degeneration. MRI can identify changes in muscle structure and composition caused by inflammation, edema, muscle damage and fat infiltration. In some embodiments, muscle strength is measured by the North Star Ambulatory Assessment. In some embodiments, muscle strength is measured by the pediatric outcomes data collection instrument (PODCI).

In some embodiments, combination treatment with the antisense oligomer, such as golodirsen, and a non-steroidal anti-inflammatory compound of the disclosure reduces muscle inflammation, reduces muscle damage, improves muscle function, and/or enhances muscle regeneration. For example, treatment may stabilize, maintain, improve, or reduce inflammation in the subject. Treatment may also, for example, stabilize, maintain, improve, or reduce muscle damage in the subject. Treatment may, for example, stabilize, maintain, or improve muscle function in the subject. In addition, for example, treatment may stabilize, maintain, improve, or enhance muscle regeneration in the subject. In some embodiments, treatment maintains, improves, or reduces muscle inflammation in the patient, as measured by, for example, magnetic resonance imaging (MRI) (e.g., MRI of the leg muscles) that would be expected without treatment.

In some embodiments, combination treatment with the antisense oligomer, such as golodirsen, and a non-steroidal anti-inflammatory compound of the disclosure results in reduced muscle inflammation in the patient relative to either the antisense oligomer or the non-steroidal anti-inflammatory compound alone. In some embodiments, combination treatment with the antisense oligomer and a non-steroidal anti-inflammatory compound of the disclosure results in reduced muscle fibrosis in the patient relative to either the antisense oligomer or the non-steroidal anti-inflammatory compound alone. In some embodiments, combination treatment with the antisense oligomer and a non-steroidal anti-inflammatory compound of the disclosure results in increased dystrophin. In some aspects, treatment results in increased dystrophin in quadricep muscle of the patient. In some aspects, treatment results in increased dystrophin in heart muscle of the patient. In some aspects, treatment results in increased dystrophin in diaphragm muscle of the patient.

In some embodiments, treatment is measured by assaying the serum of DMD patients for markers of inflammation. In some embodiments, the treatment results in a reduction in the levels of one or more, or a combination of biomarkers of inflammation. For example, in some embodiments, the biomarkers of inflammation are one or more or a combination of the following: cytokines (such as IL-1, IL-6, TNF-α), C-reactive protein (CRP), leptin, adiponectin, and creatine kinase (CK). In some embodiments, biomarkers of inflammation are assayed by methods known in the art; for example, see Rocio Cruz-Guzman et al., BioMed Research International, 2015, incorporated herein by reference. It is contemplated that treatment results in a reduction in the level of one or more of the foregoing biomarkers by at least 5%, 10%, 15%, 20%, 25%, 30%, 35%, 40%, 45%, 50%, 55%, 60%, 65%, 70%, 75%, 80%, 85%, 90%, 95%, 99%, or 100% relative to the level of the biomarker prior to treatment.

In some embodiments, treatment is measured by the 6 Minute Walk Test (6MWT). In some embodiments, treatment is measured by the 10 Meter Walk/Run Test. In various embodiments, the treatment results in a reduction or decrease in muscle inflammation in the patient. In certain embodiments, muscle inflammation in the patient is measured by MRI imaging. In some embodiments, the treatment is measured by the 4-stair climb test. In various embodiments, treatment is measured by the time to stand test. In some embodiments, treatment is measured by the North Star Ambulatory Assessment.

In some embodiments, the method of the disclosure further comprises administering to the patient a corticosteroid. In certain embodiments, the corticosteroid is Betamethasone, Budesonide, Cortisone, Dexamethasone, Hydrocortisone, Methylprednisolone, Prednisolone, Prednisone, or Deflazacort. In various embodiments, the corticosteroid is administered prior to, in conjunction with, or subsequent to administration of the antisense oligomer, such as golodirsen.

In some embodiments, the method of the disclosure further comprises confirming that the patient has a mutation in the DMD gene that is amenable to exon 53 skipping. In certain embodiments, the method of the disclosure further comprises confirming that the patient has a mutation in the DMD gene that is amenable to exon 53 skipping prior to administering the antisense oligomer, such as golodirsen.

In some embodiments, the patient has lost the ability to rise independently from supine. In some embodiments, the patient loses the ability to rise independently from supine at least one year prior to treatment with the antisense oligomer, such as golodirsen. In various embodiments, the patient loses the ability to rise independently from supine within one year of commencing treatment with the antisense oligomer. In certain embodiments, the patient loses the ability to rise independently from supine within two years of commencing treatment with the antisense oligomer.

In some embodiments, the patient maintains ambulation for at least 24 weeks after commencing treatment with the antisense oligomer, such as golodirsen. In certain embodiments, the patient has a reduction in the loss of ambulation for at least 24 weeks immediately after commencing treatment with the antisense oligomer as compared to a placebo control.

In some embodiments, dystrophin protein production is measured by reverse transcription polymerase chain reaction (RT-PCR), western blot analysis, or immunohistochemistry (IHC).

In other aspects, the disclosure provides use of the antisense oligomer, such as golodirsen, and an optional pharmaceutically acceptable carrier, in the manufacture of a medicament for treating or delaying progression of DMD in a patient, wherein the medicament comprises the antisense oligomer and an optional pharmaceutically acceptable carrier, and wherein the treatment comprises administration of the medicament in combination with edasalonexent, and an optional pharmaceutically acceptable carrier.

In other aspects, the disclosure provides the antisense oligomer, such as golodirsen, and an optional pharmaceutically acceptable carrier, for use in treating or delaying progression of DMD in a patient, wherein the treatment comprises administration of the antisense oligomer in combination with a second composition, wherein the second composition comprises edasalonexent and an optional pharmaceutically acceptable carrier.

In yet other aspects, the disclosure provides a kit comprising a container comprising edasalonexent, and an optional pharmaceutically acceptable carrier, and a package insert comprising instructions for administration of edasalonexent in combination with the antisense oligomer, such as golodirsen, an optional pharmaceutically acceptable carrier for treating or delaying progression of DMD in a patient.

In other aspects, the disclosure provides a kit which comprises a first container, a second container and a package insert, wherein the first container comprises at least one dose of a medicament comprising the antisense oligomer, such as golodirsen, the second container comprises at least one dose of a medicament comprising edasalonexent, and the package insert comprises instructions for treating a DMD patient by administration of the medicaments.

In some embodiments, the instructions provide for simultaneous administration of the antisense oligomer, such as golodirsen, and edasalonexent. In some embodiments, the instructions provide for sequential administration of the antisense oligomer and edasalonexent. In some embodiments, the instructions provide for administration of the antisense oligomer prior to administration of edasalonexent. In some embodiments, the instructions provide for administration of edasalonexent prior to administration of the antisense oligomer.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts the structure of a Phosphorodiamidate Morpholino Oligomer (PMO) and the structure of a Phosphorothioate (PSO).

FIG. 2 depicts a section of normal Dystrophin Pre-mRNA.

FIG. 3 depicts a section of abnormal Dystrophin pre-mRNA (example of DMD).

FIG. 4 depicts eteplirsen restoration of “In-frame” reading of pre-mRNA.

FIG. 5 depicts inflammation and fibrosis in muscle samples taken from the quadriceps in wild-type mice treated with saline, Mdx mice treated with saline, Mdx mice treated with CAT-1004, Mdx mice treated with the M23D PMO, and Mdx mice treated with the M23D PMO in combination with CAT-1004.

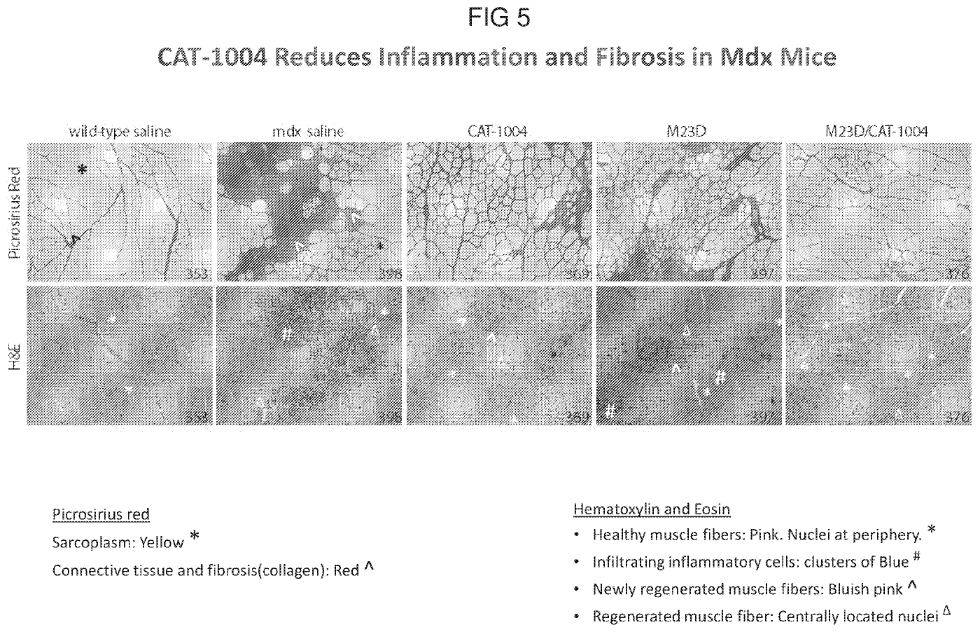

FIG. 6 graphically depicts exon skipping in mice treated with the M23D PMO and the M23D PMO in combination with CAT-1004 in quadriceps, diaphragm, and heart.

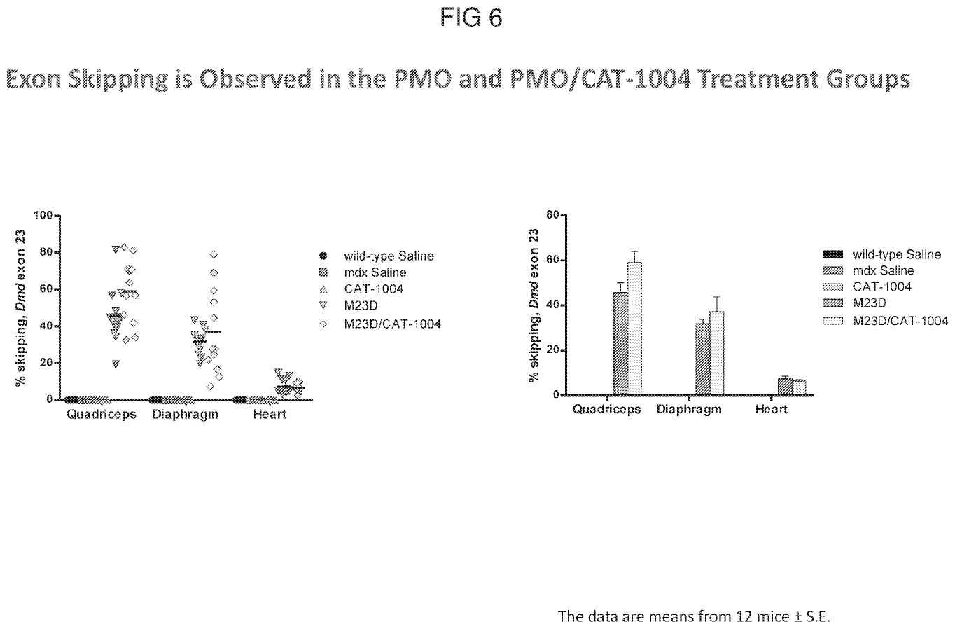

FIG. 7 depicts the levels of dystrophin in quadriceps, heart, and diaphragm treated with CAT-1004, the M23D PMO, or the M23D PMO in combination with CAT-1004.

FIG. 8 depicts the immunohistochemical analysis of dystrophin expression in quadriceps. Increased dystrophin expression was observed in mice treated with the M23D PMO in combination with CAT-1004.

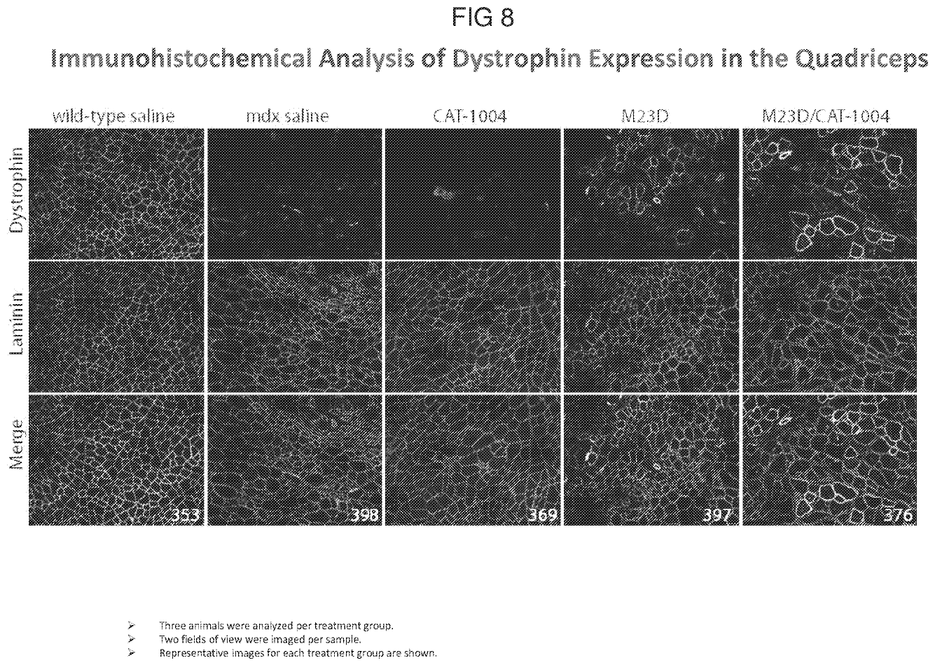

### DETAILED DESCRIPTION OF THE DISCLOSURE

The present disclosure is directed to improved methods for treating Muscular Dystrophy, such as DMD and BMD, by administering to a patient an antisense oligonucleotide that is designed to induce exon skipping in the human dystrophin pre-mRNA in combination with a non-steroidal anti-inflammatory compound. Without being bound by theory it is believed that combination therapy by administration of a dystrophin restoring agent, such as antisense oligonucleotide that is designed to induce exon skipping in the human dystrophin pre-mRNA and an NF-kB inhibitor, such as CAT-1004 may downregulate TNFα and allow for enhanced dystrophin expression in Becker muscular dystrophy patients by inhibiting TNFα-mediated increases in dystrophin regulating microRNAs (Fiorillo et al. Cell reports 2015).

Duchenne muscular dystrophy (DMD) is a rare, serious, life threatening, degenerative neuromuscular disease with a recessive X-linked inheritance. Caused by mutations in the dystrophin gene, DMD is characterized by the absence, or near absence, of functional dystrophin protein, leading to relentlessly progressive deterioration of skeletal muscle function from early childhood, and premature death, usually by 30 years of age.

To remedy this condition, the antisense compounds of the present disclosure hybridize to selected regions of a pre-processed RNA of a mutated human dystrophin gene, induce exon skipping and differential splicing in that otherwise aberrantly spliced dystrophin mRNA, and thereby allow muscle cells to produce an mRNA transcript that encodes a functional dystrophin protein. In certain embodiments, the resulting dystrophin protein is not necessarily the “wild-type” form of dystrophin, but is rather a truncated, yet functional or semi-functional, form of dystrophin.

By increasing the levels of functional dystrophin protein in muscle cells, these and related embodiments are useful in the prophylaxis and treatment of muscular dystrophy, especially those forms of muscular dystrophy, such as DMD and BMD, that are characterized by the expression of defective dystrophin proteins due to aberrant mRNA splicing.

Golodirsen, a phosphorodiamidate morpholino oligomer (PMO) which is being developed by Sarepta Therapeutics, Inc., for patients who have a confirmed mutation of exon 53 of the DMD gene has been the subject of clinical studies to test its safety and efficacy and clinical development is ongoing. The nucleobase sequence of Golodirsen has previously been described. See, for example, International Patent Application Publication No. WO 2014/153240, which is assigned to Sarepta Therapeutics, Inc.

Viltolarsen, a phosphorodiamidate morpholino oligomer (PMO) which is being developed by Nippon Shinyaku CO., LTD>, for patients who are amenable to exon 53 skipping has been the subject of clinical studies and clinical development is ongoing. The nucleobase sequence of viltolarsen has previously been described. See, for example, WHO Drug Information, Vol. 31, No. 4, 2017.

In some embodiments, dystrophin levels in muscle tissue are assessed by Western blot.

Edasalonexent belongs to a novel class of orally bioavailable NF-κB inhibitors for the treatment of dystrophic muscle. This class of compounds are composed of a polyunsaturated fatty acid (PUFA) and salicylic acid, which individually inhibit the activation of cNF-κB, conjugated together by a linker that is only susceptible to hydrolysis by intracellular fatty acid hydrolase.

Edasalonexent, [N-(2-[(4Z,7Z,10Z,13Z,16Z,19Z)-docosa-4,7,10,13,16,19-hexaenamido] ethyl)-2-hydroxybenzamide], is an orally administered small molecule in which salicylic acid and docosahexaenoic acid (DHA) are covalently conjugated through an ethylenediamine linker and that is designed to synergistically leverage the ability of both of these compounds to inhibit NF-κB. Edasalonexent was shown to significantly inhibit NF-κB p65-dependent inflammatory responses as well as downstream proinflammatory genes modulated by p65 in the golden retriever DMD model (Hammers et al., JCI Insight, 2016; 1(21):e90341). These studies also demonstrated that administration of edasalonexent, or the related analogue CAT-1041 in which DHA is replaced by eicosapentaenoic acid, reduced inflammation and fibrosis and resulted in increased exercise endurance in mdx mice and improved diaphragm function in both the mouse and dog DMD model. Edasalonexent was shown to be safe, well tolerated, and inhibited activated NF-κB pathways in a phase I clinical program that included three placebo-controlled studies in adults (see Donovan et al., The Journal of Clinical Pharmacology, 2017, 57(5), 627-637, incorporated herein by reference). Currently, a phase 1/2 clinical trial in children with DMD is under way (NCT02439216) to assess the safety and efficacy of edasalonexent.

Accordingly, the improved methods described herein may be used to reduce inflammation in a DMD patient and induce exon skipping in mutated forms of the human dystrophin gene, such as the mutated genes found in DMD and BMD, thereby treating the patient.

The methods described herein further provide improved treatment options for patients with muscular dystrophy and offer significant and practical advantages over alternate methods of treating relevant forms of muscular dystrophy. For example, in some embodiments, the improved methods relate to increased dystrophin production when an exon skipping compound (e.g., PMO) is administered in combination with an NF-kB inhibitor (e.g., CAT-1004) as compared to the administration of either agent as a monotherapy. For example, in some embodiments, the improved methods relate to administration of an antisense compound for inducing exon skipping in the human dystrophin pre-mRNA at a higher dose and/or for a longer duration than prior approaches when administered with a non-steroidal anti-inflammatory compound. In other embodiments, the improved methods relate to the administration of an antisense compound for inducing exon skipping in the human dystrophin pre-mRNA at a lower dose and/or for shorter durations than prior approaches when administered with a non-steroidal anti-inflammatory compound.

Thus, the disclosure relates to improved methods for treating muscular dystrophy such as DMD and BMD, by inducing exon skipping in a patient and reducing muscle inflammation and/or fibrosis. In some embodiments, exon skipping is induced by administering an effective amount of an antisense oligomer composition which includes a charge-neutral, phosphorodiamidate morpholino oligomer (PMO), such as golodirsen, which selectively binds to a target sequence in an exon of dystrophin pre-mRNA in combination with an effective amount of a non-steroidal anti-inflammatory compound, in particular an NF-κB inhibitor, such as edasalonexent.

In one aspect, the antisense oligomer contains a T moiety attached to the 5′ end of the antisense oligomer, wherein the T moiety is selected from:

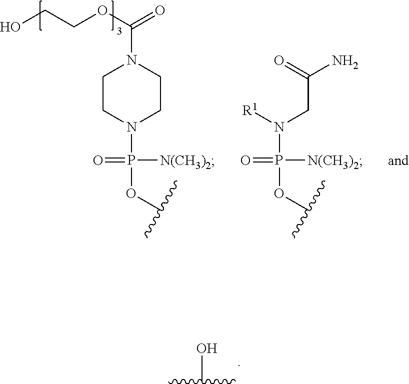

In certain embodiments, the antisense oligomer is conjugated to one or more cell-penetrating peptides (referred to herein as “CPP”). In certain embodiments, one or more CPPs are attached to a terminus of the antisense oligomer. In certain embodiments, at least one CPP is attached to the 5′ terminus of the antisense oligomer. In certain embodiments, at least one CPP is attached to the 3′ terminus of the antisense oligomer. In certain embodiments, a first CPP is attached to the 5′ terminus and a second CPP is attached to the 3′ terminus of the antisense oligomer.

In some embodiments, the CPP is an arginine-rich peptide. The term “arginine-rich” refers to a CPP having at least 2, and preferably 2, 3, 4, 5, 6, 7, or 8 arginine residues, each optionally separated by one or more uncharged, hydrophobic residues, and optionally containing about 6-14 amino acid residues. As explained herein, a CPP is preferably linked at its carboxy terminus to the 3′ and/or 5′ end of an antisense oligonucleotide through a linker, which may also be one or more amino acids, and is preferably also capped at its amino terminus by a substituent R^{a} with R^{a} selected from H, acyl, acetyl, benzoyl, or stearoyl. In some embodiments, R^{a} is acetyl.

As seen in Table 1 below, non-limiting examples of CPP's for use herein include —(RXR)₄—R^{a}, R—(FFR)₃—R^{a}, —B—X—(RXR)₄—R^{a}, —B—X—R—(FFR)₃—R^{a}, -GLY-R—(FFR)₃—R^{a}, -GLY-R₅—R^{a}, —R₅—R^{a}, -GLY-R₆—R^{a} and —R₆—R^{a}, wherein R^{a} is selected from H, acyl, benzoyl, and stearoyl, and wherein R is arginine, X is 6-aminohexanoic acid, B is β-alanine, F is phenylalanine and GLY (or G) is glycine. The CPP “R₅” is meant to indicate a peptide of five (5) arginine residues linked together via amide bonds (and not a single substituent e.g., R⁵). The CPP “R₆” is meant to indicate a peptide of six (6) arginine residues linked together via amide bonds (and not a single substituent e.g. R⁶). In some embodiments, R^{a} is acetyl.

Exemplary CPPs are provided in Table 1 (SEQ ID NOS: 11-19).

TABLE 1
Exemplary Cell-Penetrating Peptides

| Name | Sequence | SEQ ID NO: |
|---|---|---|
| (RXR)₄ | RXRRXRRXRRXR | 11 |
| (RFF)₃R | RFFRFFRFFR | 12 |
| (RXR)₄XB | RXRRXRRXRRXRXB | 13 |
| (RFF)₃RXB | RFFRFFRFFRXB | 14 |
| (RFF)₃RG | RFFRFFRFFR | 15 |
| R₅G | RRRRRG | 16 |
| R₅ | RRRRR | 17 |
| R₆G | RRRRRRG | 18 |
| R₆ | RRRRRR | 19 |

R is arginine; X is 6-aminohexanoic acid; B is β-alanine; F is phenylalanine; G is glycine

CPPs, their synthesis, and methods of conjugating to an oligomer are further described in U.S. Application Publication No. US 2012/0289457 and International Patent Application Publication Nos. WO 2004/097017, WO 2009/005793, and WO 2012/150960, the disclosures of which are incorporated herein by reference in their entirety.

In some embodiments, an antisense oligonucleotide comprises a substituent “Z,” defined as the combination of a CPP and a linker. The linker bridges the CPP at its carboxy terminus to the 3′-end and/or the 5′-end of the oligonucleotide. In various embodiments, an antisense oligonucleotide may comprise only one CPP linked to the 3′ end of the oligomer. In other embodiments, an antisense oligonucleotide may comprise only one CPP linked to the 5′ end of the oligomer.

The linker within Z may comprise, for example, 1, 2, 3, 4, or 5 amino acids.

In particular embodiments, Z is selected from:

—C(O)(CH₂)₅NH—CPP;

—C(O)(CH₂)₂NH—CPP;

—C(O)(CH₂)₂NHC(O)(CH₂)₅NH—CPP;

—C(O)CH₂NH—CPP, and the formula:

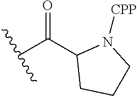

wherein the CPP is attached to the linker moiety by an amide bond at the CPP carboxy terminus.

In various embodiments, the CPP is an arginine-rich peptide as described herein and seen in Table 1. In certain embodiments, the arginine-rich CPP is —R₅-1e, (i.e., five arginine residues; SEQ ID NO: 17), wherein R^{a} is selected from H, acyl, acetyl, benzoyl, and stearoyl. In certain embodiments, R^{a} is acetyl. In various embodiments, the CPP is selected from SEQ ID NOS: 11, 12, or 1746, and the linker is selected from the group consisting of —C(O)(CH₂)₅NH—, —C(O)(CH₂)₂NH—, —C(O)(CH₂)₂NHC(O)(CH₂)₅NH—, —C(O)CH₂NH—, and

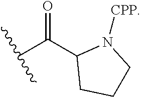

In some embodiments, the linker comprises 1, 2, 3, 4, or 5 amino acids.

In some embodiments, the CPP is SEQ ID NO: 17 and the linker is Gly. In some embodiments, the CPP is SEQ ID NO: 16.

In certain embodiments, the arginine-rich CPP is —R₆—R^{a}, (i.e., six arginine residues; SEQ ID NO: 19), wherein R^{a} is selected from H, acyl, acetyl, benzoyl, and stearoyl. In certain embodiments, R^{a} is acetyl. In various embodiments, the CPP is selected from SEQ ID NOS: 11, 12, or 19, and the linker is selected from the group consisting of —C(O)(CH₂)₅NH—, —C(O)(CH₂)₂NH—, —C(O)(CH₂)₂NHC(O)(CH₂)₅NH—, —C(O)CH₂NH—, and

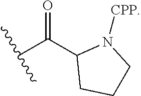

In some embodiments, the linker comprises 1, 2, 3, 4, or 5 amino acids.

In some embodiments, the CPP is SEQ ID NO: 19 and the linker is Gly. In some embodiments, the CPP is SEQ ID NO: 18.

In certain embodiments, Z is —C(O)CH₂NH—R₆—R^{a} covalently bonded to an antisense oligomer of the disclosure at the 5′ and/or 3′ end of the oligomer, wherein R^{a} is H, acyl, acetyl, benzoyl, or stearoyl to cap the amino terminus of the R₆. In certain embodiments, R^{a} is acetyl. In these non-limiting examples, the CPP is —R₆—R^{a} and the linker is —C(O)CH₂NH—, (i.e. GLY). This particular example of Z═—C(O)CH₂NH—R₆—R^{a} is also exemplified by the following structure:

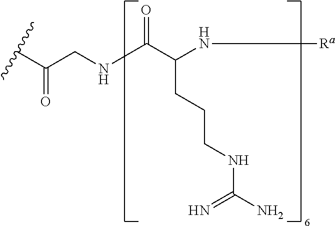

wherein R^{a} is selected from H, acyl, acetyl, benzoyl, and stearoyl.

In various embodiments, the CPP is —R₆—R^{a}, also exemplified as the following formula:

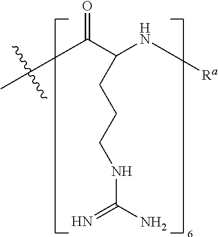

wherein R^{a} is selected from H, acyl, acetyl, benzoyl, and stearoyl. In certain embodiments, the CPP is SEQ ID NO: 18. In some embodiments, R^{a} is acetyl.

In some embodiments, the CPP is —(RXR)₄13 R^{a}, also exemplified as the following formula:

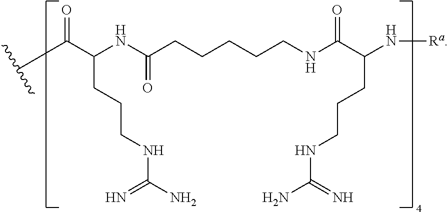

In various embodiments, the CPP is —R—(FFR)₃—R^{a}, also exemplified as the following formula:

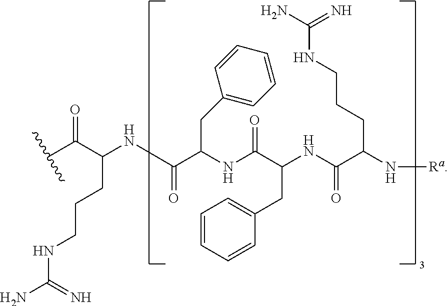

In various embodiments, Z is selected from:

—C(O)(CH₂)₅NH—CPP;

—C(O)(CH₂)₂NH—CPP;

—C(O)(CH₂)₂NHC(O)(CH₂)₅NH—CPP;

—C(O)CH₂NH—CPP; and the formula:

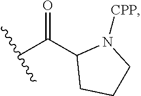

wherein the CPP is attached to the linker moiety by an amide bond at the CPP carboxy terminus, and wherein the CPP is selected from:

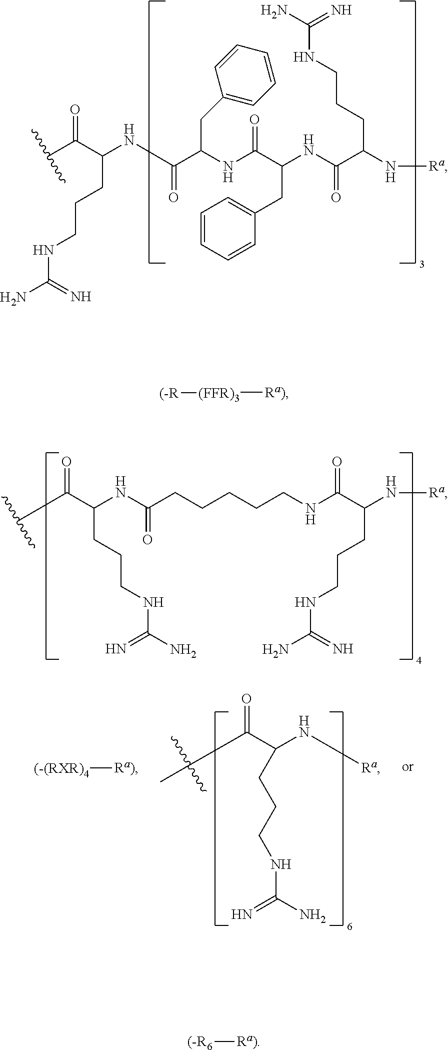

In some embodiments, R^{a} is acetyl.

A. Definitions

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

The term “alkyl,” as used herein, unless otherwise specified, refers to a saturated straight or branched hydrocarbon. In certain embodiments, the alkyl group is a primary, secondary, or tertiary hydrocarbon. In certain embodiments, the alkyl group includes one to ten carbon atoms, i.e., C₁ to C₁₀ alkyl. In certain embodiments, the alkyl group includes one to six carbon atoms, i.e., C₁ to C₆ alkyl. The term includes both substituted and unsubstituted alkyl groups, including halogenated alkyl groups. In certain embodiments, the alkyl group is a fluorinated alkyl group. Non-limiting examples of moieties with which the alkyl group can be substituted are selected from the group consisting of halogen (fluoro, chloro, bromo, or iodo), hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference. In certain embodiments, the alkyl group is selected from the group consisting of methyl, CF₃, CCl₃, CFCl₂, CF₂Cl, ethyl, CH₂CF₃, CF₂CF₃, propyl, isopropyl, butyl, isobutyl, sec-butyl, t-butyl, pentyl, isopentyl, neopentyl, hexyl, isohexyl, 3-methylpentyl, 2,2-dimethylbutyl, and 2,3-dimethylbutyl.

“Amenable to exon 53 skipping” as used herein with regard to a subject or patient is intended to include subjects and patients having one or more mutations or duplications in the dystrophin gene which, absent the skipping of exon 53 of the dystrophin pre-mRNA, either causes the reading frame to be out-of-frame thereby disrupting translation of the pre-mRNA, or causes transcription of the duplicate exon, leading to an inability of the subject or patient to produce functional or semi-functional dystrophin. Determining whether a patient has a mutation in the dystrophin gene that is amenable to exon skipping is well within the purview of one of skill in the art (see, e.g., Aartsma-Rus et al. (2009) Hum Mutat. 30:293-299; Gurvich et al., Hum Mutat. 2009; 30(4) 633-640; and Fletcher et al. (2010) Molecular Therapy 18(6) 1218-1223.).

The terms “antisense oligomer” and “antisense compound” and “antisense oligonucleotide” and “oligomer” and “oligonucleotide” are used interchangeably in this disclosure and refer to a sequence of subunits connected by intersubunit linkages. Each subunit consists of: (i) a ribose sugar or a derivative thereof; and (ii) a nucleobase bound thereto, such that the order of the base-pairing moieties forms a base sequence that is complementary to a target sequence in a nucleic acid (typically an RNA) by Watson-Crick base pairing, to form a nucleic acid:oligomer heteroduplex within the target sequence with the proviso that either the subunit, the intersubunit linkage, or both are not naturally occurring. In certain embodiments, the oligomer is a PMO. In other embodiments, the antisense oligonucleotide is a 2′-O-methyl phosphorothioate. In other embodiments, the antisense oligonucleotide of the disclosure is a peptide nucleic acid (PNA), a locked nucleic acid (LNA), or a bridged nucleic acid (BNA) such as 2′-O,4′-C-ethylene-bridged nucleic acid (ENA). Additional exemplary embodiments are described.

The terms “morpholino,” “morpholino oligomer,” or “PMO” refer to a phosphorodiamidate morpholino oligomer of the following general structure:

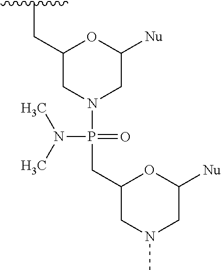

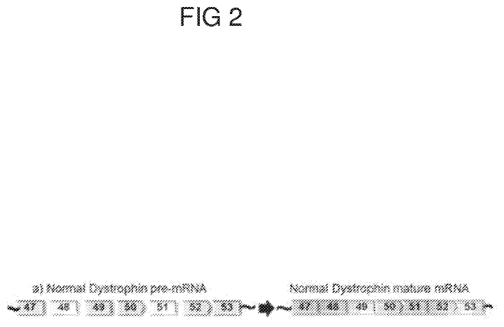

and as described in FIG. 2 of Summerton, J., et al., Antisense & Nucleic Acid Drug Development, 7: 187-195 (1997). Morpholinos as described herein are intended to cover all stereoisomers and configurations of the foregoing general structure. The synthesis, structures, and binding characteristics of morpholino oligomers are detailed in U.S. Pat. Nos. 5,698,685, 5,217,866, 5,142,047, 5,034,506, 5,166,315, 5,521,063, 5,506,337, 8,076,476, and 8,299,206, all of which are incorporated herein by reference.

In certain embodiments, a morpholino is conjugated at the 5′ or 3′ end of the oligomer with a “tail” moiety to increase its stability and/or solubility. Exemplary tails include:

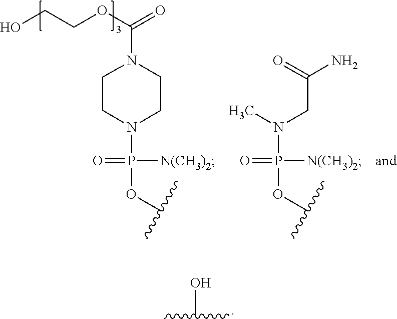

Of the above exemplary tail moieties, “TEG” or “EG3” refers to the following tail moiety:

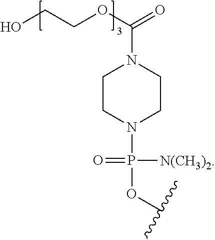

Of the above exemplary tail moieties, “GT” refers to the following tail moiety:

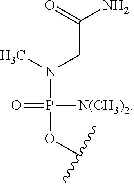 [SMILES: *OP(=O)(N(C)C)N(C)CC(N)=O]

As used herein, the terms “-G-R₅” and “-G-R₅—Ac” are used interchangeably and refer to a peptide moiety conjugated to an antisense oligomer of the disclosure. In various embodiments, “G” represents a glycine residue conjugated to “R₅” by an amide bond, and each “R” represents an arginine residue conjugated together by amide bonds such that “R₅” means five (5) arginine residues conjugated together by amide bonds. The arginine residues can have any stereo configuration, for example, the arginine residues can be L-arginine residues, D-arginine residues, or a mixture of D- and L-arginine residues. In certain embodiments, “-G-R₅” or “-G-R₅—Ac” is linked to the distal —OH or NH₂ of the “tail” moiety. In certain embodiments, “-G-R₅” or “-G-R₅—Ac” is conjugated to the morpholine ring nitrogen of the 3′ most morpholino subunit of a PMO antisense oligomer of the disclosure. In some embodiments, “-G-R₅” or “-G-R₅—Ac” is conjugated to the 3′ end of an antisense oligomer of the disclosure and is of the following formula:

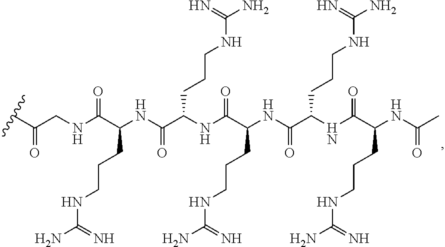 [SMILES: *C(=O)CNC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(C)=O]

or a pharmaceutically acceptable salt thereof, or

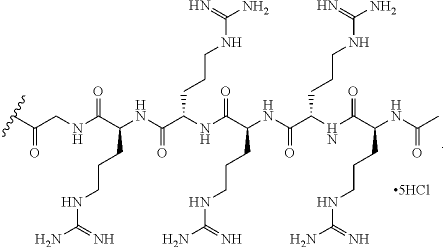 [SMILES: *C(=O)CNC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(C)=O]

As used herein, the terms “-G-R₆” and “-G-R₆—Ac” are used interchangeably and refer to a peptide moiety conjugated to an antisense oligomer of the disclosure. In various embodiments, “G” represents a glycine residue conjugated to “R₆” by an amide bond, and each “R” represents an arginine residue conjugated together by amide bonds such that “R₆” means six (6) arginine residues conjugated together by amide bonds. The arginine residues can have any stereo configuration, for example, the arginine residues can be L-arginine residues, D-arginine residues, or a mixture of D- and L-arginine residues. In certain embodiments, “-G-R₆” or “-G-R₆—Ac” is linked to the distal —OH or NH₂ of the “tail” moiety. In certain embodiments, “-G-R₆” or “-G-R₆—Ac” is conjugated to the morpholine ring nitrogen of the 3′ most morpholino subunit of a PMO antisense oligomer of the disclosure. In some embodiments, “-G-R₆” or “-G-R₆—Ac” is conjugated to the 3′ end of an antisense oligomer of the disclosure and is of the following formula:

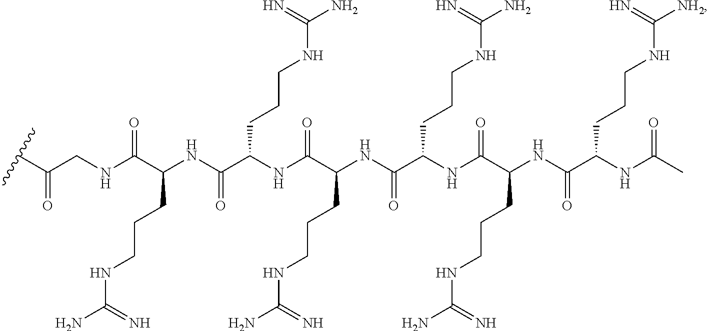 [SMILES: *C(=O)CNC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(C)=O]

or a pharmaceutically acceptable salt thereof, or

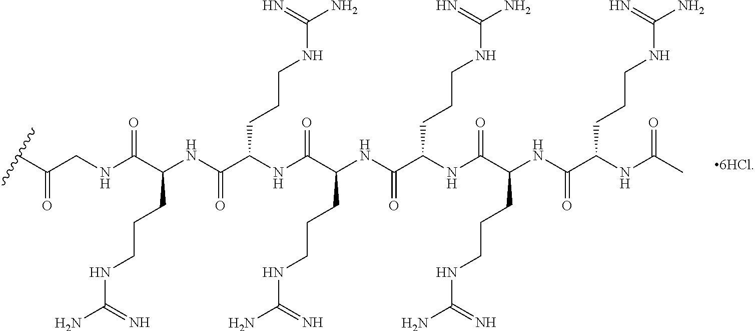 [SMILES: *C(=O)CNC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(C)=O]

The terms “nucleobase” (Nu), “base pairing moiety” or “base” are used interchangeably to refer to a purine or pyrimidine base found in naturally occurring, or “native” DNA or RNA (e.g., uracil, thymine, adenine, cytosine, and guanine), as well as analogs of these naturally occurring purines and pyrimidines, that may confer improved properties, such as binding affinity to the oligomer. Exemplary analogs include hypoxanthine (the base component of inosine); 2,6-diaminopurine; 5-methyl cytosine; C5-propynyl-modified pyrimidines; 10-(9-(aminoethoxy)phenoxazinyl) (G-clamp) and the like.

Further examples of base pairing moieties include, but are not limited to, uracil, thymine, adenine, cytosine, guanine and hypoxanthine (inosine) having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). The modified nucleobases disclosed in Chiu and Rana, RNA, 2003, 9, 1034-1048, Limbach et al. Nucleic Acids Research, 1994, 22, 2183-2196 and Revankar and Rao, Comprehensive Natural Products Chemistry, vol. 7, 313, are also contemplated, the contents of which are incorporated herein by reference.

Further examples of base pairing moieties include, but are not limited to, expanded-size nucleobases in which one or more benzene rings has been added. Nucleic base replacements described in the Glen Research catalog (www.glenresearch.com); Krueger A T et al., Acc. Chem. Res., 2007, 40, 141-150; Kool, E T, Acc. Chem. Res., 2002, 35, 936-943; Benner S. A., et al., Nat. Rev. Genet., 2005, 6, 553-543; Romesberg, F. E., et al., Curr. Opin. Chem. Biol., 2003, 7, 723-733; Hirao, I., Curr. Opin. Chem. Biol., 2006, 10, 622-627, the contents of which are incorporated herein by reference, are contemplated as useful for the synthesis of the oligomers described herein. Examples of expanded-size nucleobases are shown below:

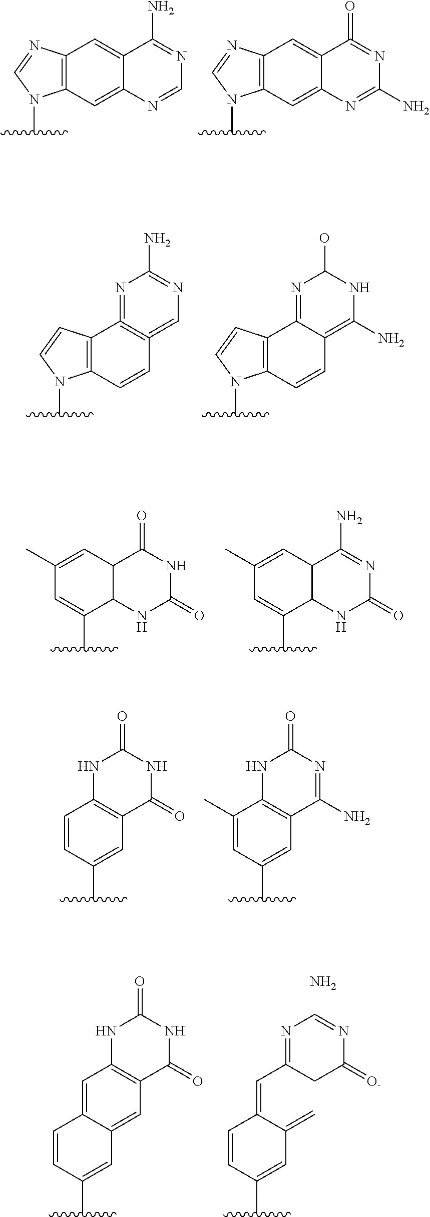

“Golodirsen”, also known by its code name “SRP-4053” is a PMO having the base sequence 5′- GTTGCCTCCGGTTCTGAAGGTGTTC-3′ (SEQ ID NO:1). Golodirsen is registered under CAS Registry Number 1422959-91-8. Chemical names include: all-P-ambo-[P,2′,3′-trideoxy-P-(dimethylamino)-2′,3′-imino-2′,3′-seco](2′a□5′)(G-T-T-G-C-C-T-C-C-G-G-T-T-C-T-G-A-A-G-G-T-G-T-T-C) 5′-[4-({2-[2-(2-hydroxyethoxy)ethoxy]ethoxy}carbonyl)-N,N-dimethylpiperazine-1-phosphonamidate]

Golodirsen has the following structure:

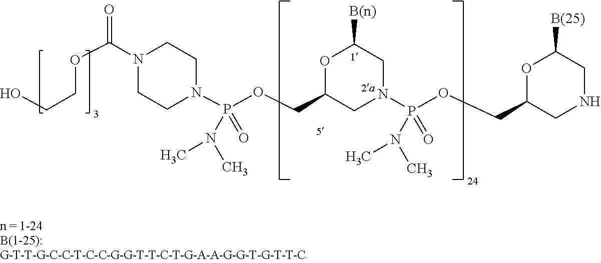

And also is represented by the following chemical structure:

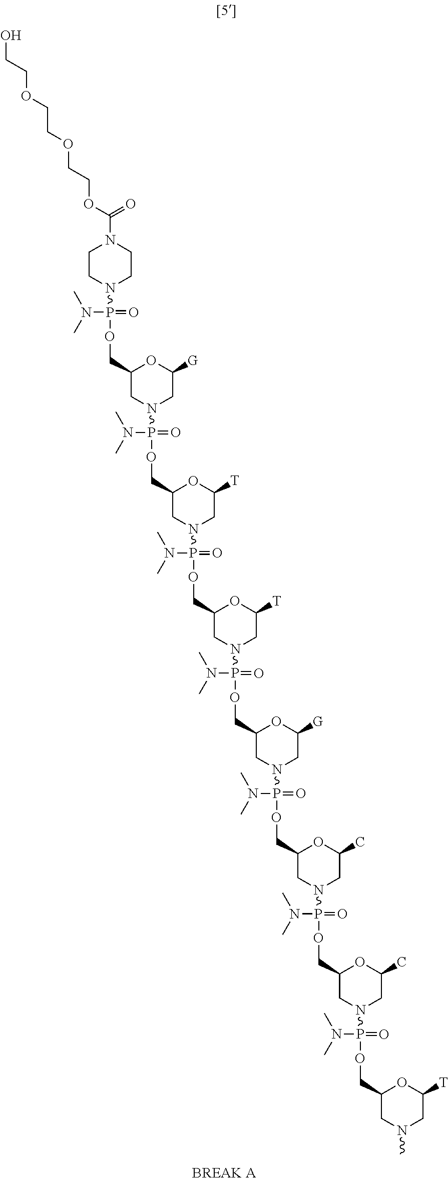

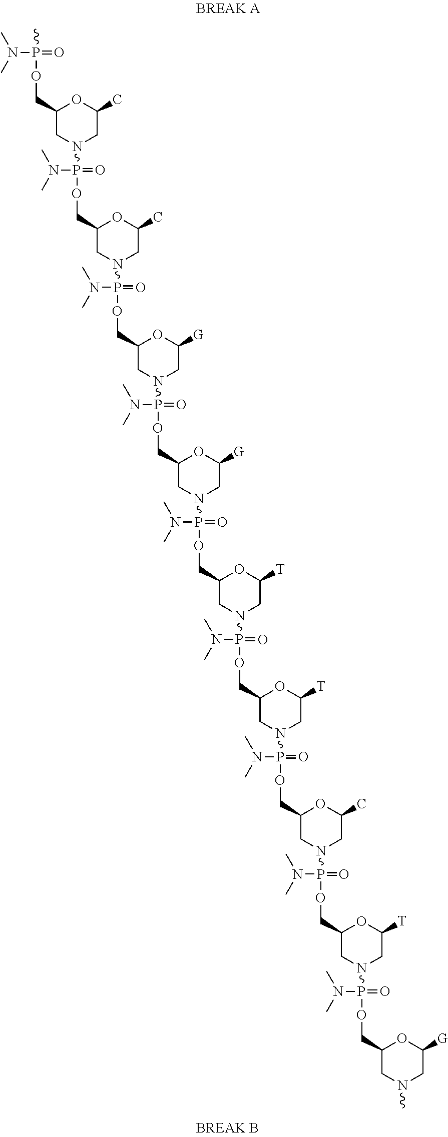

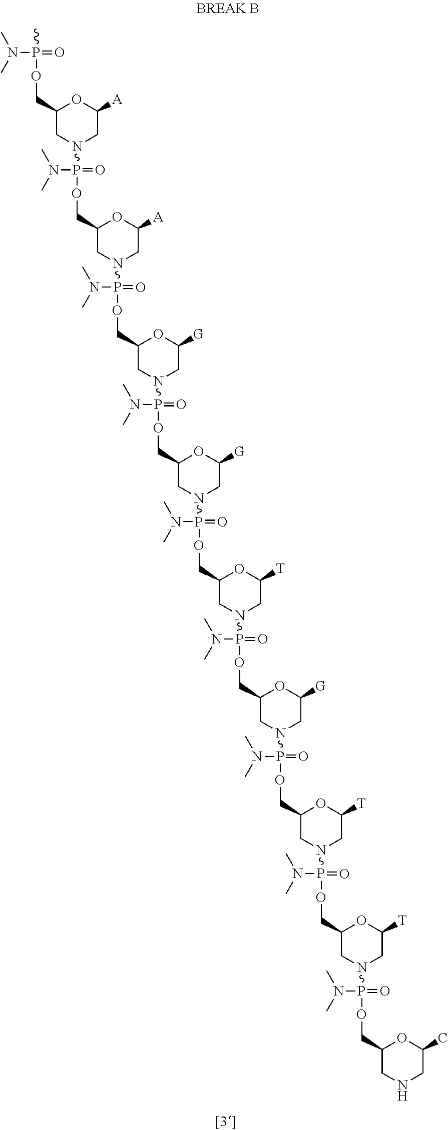

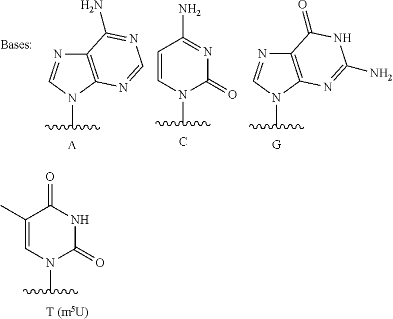

The sequence of bases from the 5′ end to the 3′ end is:

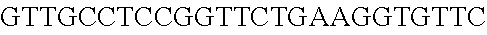

For clarity, structures of the disclosure including, for example, the above Formula, are continuous from 5′ to 3′, and, for the convenience of depicting the entire structure in a compact form, various illustration breaks labeled “BREAK A” and “BREAK B,” have been included. As would be understood by the skilled artisan, for example, each indication of “BREAK A” shows a continuation of the illustration of the structure at these points. The skilled artisan understands that the same is true for each instance of “BREAK A” and for “BREAK B” in the structures above. None of the illustration breaks, however, are intended to indicate, nor would the skilled artisan understand them to mean, an actual discontinuation of the structure above.

“Viltolarsen”, also known by its code name “NS-065” is a PMO having the base sequence 5′-CCTCCGGTTCTGAAGGTGTTC-3′ (SEQ ID NO: 20). Viltolarsen is registered under CAS Registry Number 2055732-84-6. Chemical names include: all-P-ambo-[2′,3′-azanediyl-P,2′,3′-trideoxy-P-(dimethylamino)-2′,3′-seco](2′-N→5′)(CCTCCGGTTCTGAAGGTGTTC).

Viltolarsen has the following structure:

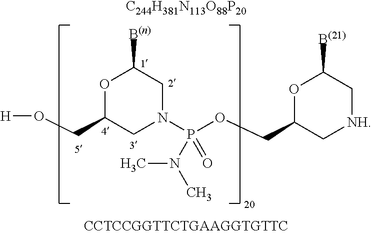

As used herein, a set of brackets used within a structural formula indicate that the structural feature between the brackets is repeated. In some embodiments, the brackets used can be “[” and “],” and in certain embodiments, brackets used to indicate repeating structural features can be “(” and “).” In some embodiments, the number of repeat iterations of the structural feature between the brackets is the number indicated outside the brackets such as 2, 3, 4, 5, 6, 7, and so forth. In various embodiments, the number of repeat iterations of the structural feature between the brackets is indicated by a variable indicated outside the brackets such as “Z”.

As used herein, a bond draw to chiral carbon or phosphorous atom within a straight bond or a squiggly bond structural formula indicates that the stereochemistry of the chiral carbon or phosphorous is undefined and is intended to include all forms of the chiral center. Examples of such illustrations are depicted below:

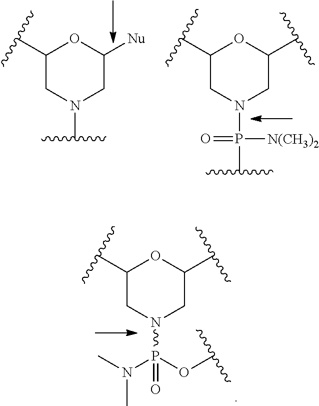

As used herein, the term “M23D” means AVI-4225, which is a PMO which hybridizes to mouse dystrophin exon 23 pre-mRNA having a TEG tail moiety at the 5′ end and which has the sequence GGC CAA ACC TCG GCT TAC CTG AAA T (SEQ ID NO: 10).

The term “non-steroidal anti-inflammatory compound” refers to an anti-inflammatory compound or drug that is not a steroid, corticosteroid, glucocorticoid, anabolic steroid or mineralcorticoid. In certain embodiments, non-steroidal anti-inflammatory compounds are NF-κB inhibitors. In some embodiments, an NF-kB inhibitor is composed of a polyunsaturated fatty acid (PUFA) and salicylic acid. In some embodiments, the NF-kB inhibitor is CAT-1004 or CAT-1041. The term “CAT-1004” is used interchangeably with the term “edasalonexent” [N-(2-[(4Z,7Z,10Z,13Z,16Z,19Z)-docosa-4,7,10,13,16,19-hexaenamido] ethyl)-2-hydroxybenzamide].

“Dystrophin” is a rod-shaped cytoplasmic protein, and a vital part of the protein complex that connects the cytoskeleton of a muscle fiber to the surrounding extracellular matrix through the cell membrane. Dystrophin contains multiple functional domains. For instance, dystrophin contains an actin binding domain at about amino acids 14-240 and a central rod domain at about amino acids 253-3040. This large central domain is formed by 24 spectrin-like triple-helical elements of about 109 amino acids, which have homology to alpha-actinin and spectrin. The repeats are typically interrupted by four proline-rich non-repeat segments, also referred to as hinge regions. Repeats 15 and 16 are separated by an 18 amino acid stretch that appears to provide a major site for proteolytic cleavage of dystrophin. The sequence identity between most repeats ranges from 10-25%. One repeat contains three alpha-helices: 1, 2 and 3. Alpha-helices 1 and 3 are each formed by 7 helix turns, probably interacting as a coiled-coil through a hydrophobic interface. Alpha-helix 2 has a more complex structure and is formed by segments of four and three helix turns, separated by a Glycine or Proline residue. Each repeat is encoded by two exons, typically interrupted by an intron between amino acids 47 and 48 in the first part of alpha-helix 2. The other intron is found at different positions in the repeat, usually scattered over helix-3. Dystrophin also contains a cysteine-rich domain at about amino acids 3080-3360), including a cysteine-rich segment (i.e., 15 Cysteines in 280 amino acids) showing homology to the C-terminal domain of the slime mold (Dictyostelium discoideum) alpha-actinin. The carboxy-terminal domain is at about amino acids 3361-3685.

The amino-terminus of dystrophin binds to F-actin and the carboxy-terminus binds to the dystrophin-associated protein complex (DAPC) at the sarcolemma. The DAPC includes the dystroglycans, sarcoglycans, integrins and caveolin, and mutations in any of these components cause autosomally inherited muscular dystrophies. The DAPC is destabilized when dystrophin is absent, which results in diminished levels of the member proteins, and in turn leads to progressive fibre damage and membrane leakage. In various forms of muscular dystrophy, such as Duchenne's muscular dystrophy (DMD) and Becker's muscular dystrophy (BMD), muscle cells produce an altered and functionally defective form of dystrophin, or no dystrophin at all, mainly due to mutations in the gene sequence that lead to incorrect splicing. The predominant expression of the defective dystrophin protein, or the complete lack of dystrophin or a dystrophin-like protein, leads to rapid progression of muscle degeneration, as noted above. In this regard, a “defective” dystrophin protein may be characterized by the forms of dystrophin that are produced in certain subjects with DMD or BMD, as known in the art, or by the absence of detectable dystrophin.

An “exon” refers to a defined section of nucleic acid that encodes for a protein, or a nucleic acid sequence that is represented in the mature form of an RNA molecule after either portions of a pre-processed (or precursor) RNA have been removed by splicing. The mature RNA molecule can be a messenger RNA (mRNA) or a functional form of a non-coding RNA, such as rRNA or tRNA. The human dystrophin gene has about 79 exons.

An “intron” refers to a nucleic acid region (within a gene) that is not translated into a protein. An intron is a non-coding section that is transcribed into a precursor mRNA (pre-mRNA), and subsequently removed by splicing during formation of the mature RNA.

An “effective amount” or “therapeutically effective amount” refers to an amount of therapeutic compound, such as an antisense oligonucleotide or a non-steroidal anti-inflammatory compound, that when administered to a human subject, either as a single dose or as part of a series of doses, is effective to produce a desired therapeutic effect.

For an antisense oligonucleotide, this effect is typically brought about by inhibiting translation or natural splice-processing of a selected target sequence, or producing a clinically meaningful amount of dystrophin (statistical significance). In some embodiments, an effective amount is at least 10 mg/kg or at least 20 mg/kg of a composition including an antisense oligonucleotide for a period of time to treat the subject. In some embodiments, an effective amount is at least 10 mg/kg or at least 20 mg/kg of a composition including an antisense oligonucleotide to increase the dystrophin levels in a subject, as measured by, for example, the percent normal dystrophin in a subject following treatment relative to baseline dystrophin levels prior to treatment. In certain embodiments, an effective amount is at least 10 mg/kg or at least 20 mg/kg of a composition including an antisense oligonucleotide to stabilize, maintain, or improve walking distance from a 20% deficit, for example in a 6 MWT, in a patient, relative to a healthy peer. In various embodiments, an effective amount is is at least 10 mg/kg to about 20 mg/kg, at least 20 mg/kg to about 30 mg/kg, about 25 mg/kg to about 30 mg/kg, or about 30 mg/kg to about 50 mg/kg. In some embodiments, an effective amount is about 30 mg/kg or about 50 mg/kg. In another aspect, an effective amount is at least 20 mg/kg, about 25 mg/kg, about 30 mg/kg, or about 30 mg/kg to about 50 mg/kg, for at least 24 weeks, at least 36 weeks, or at least 48 weeks, to thereby increase the dystrophin levels in a subject, as measured by, for example, the percent normal dystrophinin a subject following treatment relative to baseline dystrophin levels prior to treatment, and stabilize or improve walking distance from a 20% deficit, for example in a 6 MWT, in the patient relative to a healthy peer. In some embodiments, treatment increases the percent normal dystrophin to 0.01-0.05%, 0.01-0.1%, 0.01-0.15%, 0.01-0.2%, 0.01-0.25%, 0.01-0.28%, 0.01-0.3%, 0.01-0.35%, 0.01-0.4%, 0.01-0.45%, 0.01-0.5%, 0.01-0.6%, 0.01-0.7%, 0.01-0.8%, 0.01-0.9%, 0.01-1%, 0.01-1.25%, 0.01-1.5%, 0.01-2%, 0.01-2.5%, 0.03-0.05%, 0.03-0.1%, 0.03-0.15%, 0.03-0.2%, 0.03-0.25%, 0.03-0.28%, 0.03-0.3%, 0.03-0.35%, 0.03-0.4%, 0.03-0.45%, 0.03-0.5%, 0.03-0.6%, 0.03-0.7%, 0.03-0.8%, 0.03-0.9%, 0.03-1%, 0.03-1.25%, 0.03-1.5%, 0.03-2%, 0.03-2.5%, 0.05-0.1%, 0.05-0.15%, 0.05-0.2%, 0.05-0.25%, 0.05-0.28%, 0.05-0.3%, 0.05-0.35%, 0.05-0.4%, 0.05-0.45%, 0.05-0.5%, 0.05-0.6%, 0.05-0.7%, 0.05-0.8%, 0.05-0.9%, 0.05-1%, 0.05-1.25%, 0.05-1.5%, 0.05-2%, 0.05-2.5%, 0.1-0.15%, 0.1-0.2%, 0.1-0.25%, 0.1-0.28%, 0.1-0.3%, 0.1-0.35%, 0.1-0.4%, 0.1-0.45%, 0.1-0.5%, 0.1-0.6%, 0.1-0.7%, 0.1-0.8%, 0.1-0.9%, 0.1-1%, 0.1-1.25%, 0.1-1.5%, 0.1-2%, 0.1-2.5%, 0.2-0.25%, 0.2-0.28%, 0.2-0.3%, 0.2-0.35%, 0.2-0.4%, 0.2-0.45%, 0.2-0.5%, 0.2-0.6%, 0.2-0.7%, 0.2-0.8%, 0.2-0.9%, 0.2-1%, 0.2-1.25%, 0.2-1.5%, 0.2-2%, 0.2-2.5%, 0.25-0.3%, 0.25-0.35%, 0.25-0.4%, 0.25-0.45%, 0.25-0.5%, 0.25-0.6%, 0.25-0.7%, 0.25-0.8%, 0.25-0.9%, 0.25-1%, 0.25-1.25%, 0.25-1.5%, 0.25-2%, 0.25-2.5%, 0.3-0.35%, 0.3-0.4%, 0.3-0.45%, 0.3-0.5%, 0.3-0.6%, 0.3-0.7%, 0.3-0.8%, 0.3-0.9%, 0.3-1%, 0.3-1.25%, 0.3-1.5%, 0.3-2%, 0.3-2.5%, 0.4-0.5%, 0.4-0.6%, 0.4-0.7%, 0.4-0.8%, 0.4-0.9%, 0.4-1%, 0.4-1.25%, 0.4-1.5%, 0.4-2%, 0.4-2.5%, 0.5-0.6%, 0.5-0.7%, 0.5-0.8%, 0.5-0.9%, 0.5-1%, 0.5-1.25%, 0.5-1.5%, 0.5-2%, 0.5-2.5%, 1-2%, 1-2.5%, 2-2.5%, 1-3%, 1-5%, 2-3%, 2-5%, 5-10%, 10-20%, 20-60%, or 30-50% in the patient.

In some embodiments, the antisense oligomers of the present disclosure are administered in doses generally from about 10-160 mg/kg or 20-160 mg/kg. In some cases, doses of greater than 160 mg/kg may be necessary. In some embodiments, doses for i.v. administration are from about 0.5 mg to 160 mg/kg. In some embodiments, the antisense oligomer conjugates are administered at doses of about 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg. In some embodiments, the antisense oligomer conjugates are administered at doses of about 10 mg/kg, 11 mg/kg, 12 mg/kg, 15 mg/kg, 18 mg/kg, 20 mg/kg, 21 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg 50 mg/kg, 51 mg/kg, 52 mg/kg, 53 mg/kg, 54 mg/kg, 55 mg/kg, 56 mg/kg, 57 mg/kg, 58 mg/kg, 59 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, including all integers in between. In some embodiments, the oligomer is administered at 10 mg/kg. In some embodiments, the oligomer is administered at 20 mg/kg. In some embodiments, the oligomer is administered at 30 mg/kg. In some embodiments, the oligomer is administered at 40 mg/kg. In some embodiments, the oligomer is administered at 60 mg/kg. In some embodiments, the oligomer is administered at 80 mg/kg. In some embodiments, the oligomer is administered at 160 mg/kg. In some embodiments, the oligomer is administered at 50 mg/kg.

In some embodiments, treatment increases sarcolemma-associated dystrophin protein expression and distribution.

For non-steroidal anti-inflammatory compounds, this effect is typically brought about by reducing inflammation, muscle mass, muscle density and/or enhancing muscle regeneration. In some embodiments, an effective amount of the non-steroidal anti-inflammatory compound is between about 10 mg/kg and about 1000 mg/kg, one to three times per day, once every other day, once per week, biweekly, once per month, or bimonthly. In some embodiments, an effective amount is about 33 mg/kg, about 67 mg/kg, or about 100 mg/kg, one to three times per day, once every other day, once per week, biweekly, once per month, or bimonthly.

As used herein, the terms “function” and “functional” and the like refer to a biological, enzymatic, or therapeutic function.

A “functional” dystrophin protein refers generally to a dystrophin protein having sufficient biological activity to reduce the progressive degradation of muscle tissue that is otherwise characteristic of muscular dystrophy, typically as compared to the altered or “defective” form of dystrophin protein that is present in certain subjects with DMD or BMD. In certain embodiments, a functional dystrophin protein may have about 10%, 20%, 30%, 40%, 50%, 60%, 70%, 80%, 90%, or 100% (including all integers in between) of the in vitro or in vivo biological activity of wild-type dystrophin, as measured according to routine techniques in the art. As one example, dystrophin-related activity in muscle cultures in vitro can be measured according to myotube size, myofibril organization (or disorganization), contractile activity, and spontaneous clustering of acetylcholine receptors (see, e.g., Brown et al., Journal of Cell Science. 112:209-216, 1999). Animal models are also valuable resources for studying the pathogenesis of disease, and provide a means to test dystrophin-related activity. Two of the most widely used animal models for DMD research are the mdx mouse and the golden retriever muscular dystrophy (GRMD) dog, both of which are dystrophin negative (see, e.g., Collins & Morgan, Int J Exp Pathol 84: 165-172, 2003). These and other animal models can be used to measure the functional activity of various dystrophin proteins. Included are truncated forms of dystrophin, such as those forms that are produced by certain of the exon-skipping antisense compounds of the present disclosure.

The terms “induction” or “restoration” of dystrophin synthesis or production refers generally to the production of a dystrophin protein including truncated forms of dystrophin in a patient with muscular dystrophy following treatment with an antisense oligonucleotide as described herein. In some embodiments, treatment results in an increase in novel dystrophin production in a patient by 0.1%, 0.5%, 1%, 1.5%, 2%, 2.5%, 3%, 3.5%, 4%, 4.5%, 5%, 10%, 20%, 30%, 40%, or 50%, (including all integers in between). In some embodiments, treatment results in an increase in novel dystrophin production in a patient by about 0.01-0.05%, 0.01-0.1%, 0.01-0.15%, 0.01-0.2%, 0.01-0.25%, 0.01-0.28%, 0.01-0.3%, 0.01-0.35%, 0.01-0.4%, 0.01-0.45%, 0.01-0.5%, 0.01-0.6%, 0.01-0.7%, 0.01-0.8%, 0.01-0.9%, 0.01-1%, 0.01-1.25%, 0.01-1.5%, 0.01-2%, 0.01-2.5%, 0.03-0.05%, 0.03-0.1%, 0.03-0.15%, 0.03-0.2%, 0.03-0.25%, 0.03-0.28%, 0.03-0.3%, 0.03-0.35%, 0.03-0.4%, 0.03-0.45%, 0.03-0.5%, 0.03-0.6%, 0.03-0.7%, 0.03-0.8%, 0.03-0.9%, 0.03-1%, 0.03-1.25%, 0.03-1.5%, 0.03-2%, 0.03-2.5%, 0.05-0.1%, 0.05-0.15%, 0.05-0.2%, 0.05-0.25%, 0.05-0.28%, 0.05-0.3%, 0.05-0.35%, 0.05-0.4%, 0.05-0.45%, 0.05-0.5%, 0.05-0.6%, 0.05-0.7%, 0.05-0.8%, 0.05-0.9%, 0.05-1%, 0.05-1.25%, 0.05-1.5%, 0.05-2%, 0.05-2.5%, 0.1-0.15%, 0.1-0.2%, 0.1-0.25%, 0.1-0.28%, 0.1-0.3%, 0.1-0.35%, 0.1-0.4%, 0.1-0.45%, 0.1-0.5%, 0.1-0.6%, 0.1-0.7%, 0.1-0.8%, 0.1-0.9%, 0.1-1%, 0.1-1.25%, 0.1-1.5%, 0.1-2%, 0.1-2.5%, 0.2-0.25%, 0.2-0.28%, 0.2-0.3%, 0.2-0.35%, 0.2-0.4%, 0.2-0.45%, 0.2-0.5%, 0.2-0.6%, 0.2-0.7%, 0.2-0.8%, 0.2-0.9%, 0.2-1%, 0.2-1.25%, 0.2-1.5%, 0.2-2%, 0.2-2.5%, 0.25-0.3%, 0.25-0.35%, 0.25-0.4%, 0.25-0.45%, 0.25-0.5%, 0.25-0.6%, 0.25-0.7%, 0.25-0.8%, 0.25-0.9%, 0.25-1%, 0.25-1.25%, 0.25-1.5%, 0.25-2%, 0.25-2.5%, 0.3-0.35%, 0.3-0.4%, 0.3-0.45%, 0.3-0.5%, 0.3-0.6%, 0.3-0.7%, 0.3-0.8%, 0.3-0.9%, 0.3-1%, 0.3-1.25%, 0.3-1.5%, 0.3-2%, 0.3-2.5%, 0.4-0.5%, 0.4-0.6%, 0.4-0.7%, 0.4-0.8%, 0.4-0.9%, 0.4-1%, 0.4-1.25%, 0.4-1.5%, 0.4-2%, 0.4-2.5%, 0.5-0.6%, 0.5-0.7%, 0.5-0.8%, 0.5-0.9%, 0.5-1%, 0.5-1.25%, 0.5-1.5%, 0.5-2%, 0.5-2.5%, 1-2%, 1-2.5%, 2-2.5%, 1-3%, 1-5%, 2-3%, 2-5%, 5-10%, 10-20%, 20-60%, or 30-50%.

In some embodiments, treatment increases the percent normal dystrophin to at least 0.01%, about 0.02%, about 0.03%, about 0.04%, about 0.05%, about 0.06%, about 0.07%, about 0.08%, about 0.09%, about 0.1%, about 0.2%, about 0.25%, about 0.28%, about 0.3%, about 0.4%, about 0.5%, about 1%, about 1.5%, about 2%, about 2.5%, about 3%, about 3.5%, about 4%, about 4.5, about 5%, about 10%, about 15%, about 20%, about 30%, about 40%, or about 50% in the subject. In other embodiments, treatment increases the percent normal dystrophin to about 0.01% to about 0.1%, about 0.01% to about 0.2%, about 0.01% to about 0.3%, about 0.01% to about 0.04%, about 0.01% to about 0.05%, about 0.1% to about 1%, about 0.01% to about 0.15%, about 0.5° A to about 1%, about 1° A to about 1.5%, 1° A to about 2%, about 1° A to about 2.5%, about 1.5% to about 2.5%, about 0.5% to about 2.5%, about 0.5% to about 5%, about 1% to about 5%, or about 1% to about 10% of the subject. In some embodiments, treatment increases sarcolemma-associated dystrophin protein expression and distribution. The percent normal dystrophin and/or sarcolemma-associated dystrophin protein expression and distribution in a patient following treatment can be determined following muscle biopsy using known techniques, such as Western blot analysis. For example, a muscle biopsy may be taken from a suitable muscle, such as the biceps brachii muscle in a patient. Analysis of the levels of dystrophin and/or sarcolemma-associated dystrophin protein expression and distribution may be performed pre-treatment and/or post-treatment or at time points throughout the course of treatment. In some embodiments, a post-treatment biopsy is taken from the contralateral muscle from the pre-treatment biopsy. Pre- and post-treatment dystrophin expression studies may be performed using any suitable assay for dystrophin. In some embodiments, immunohistochemical detection is performed on tissue sections from the muscle biopsy using an antibody that is a marker for dystrophin, such as a monoclonal or a polyclonal antibody. For example, the MANDYS106 antibody can be used which is a highly sensitive marker for dystrophin. Any suitable secondary antibody may be used.

In some embodiments, the levels of dystrophin are determined by Western blot analysis. Normal muscle samples have 100% dystrophin. Therefore, the levels of dystrophin can be expressed as a percentage of normal. To control for the presence of trace levels of dystrophin in the pretreatment muscle as well as revertant muscle a baseline can be set using pre-treatment muscles from each patient when determining percent normal dystrophin in post-treatment muscles. This may be used as a threshold for determining percent normal dystrophin in post-treatment muscle in that patient. In some embodiments, Western blot analysis with monoclonal or polyclonal anti-dystrophin antibodies can be used to determine the percent normal dystrophin. For example, the anti-dystrophin antibody NCL-Dysl from Novacastra may be used. The percent normal dystrophincan also be analyzed by determining the expression of the components of the sarcoglycan complex (β,γ) and/or neuronal NOS.

In some embodiments, treatment with an antisense oligonucleotide of the disclosure, such as golodirsen, slows or reduces the progressive respiratory muscle dysfunction and/or failure in patients with DMD that would be expected without treatment. In some embodiments, treatment with an antisense oligonucleotide of the disclosure may reduce or eliminate the need for ventilation assistance that would be expected without treatment. In some embodiments, measurements of respiratory function for tracking the course of the disease, as well as the evaluation of potential therapeutic interventions include Maximum inspiratory pressure (MIP), maximum expiratory pressure (MEP) and forced vital capacity (FVC). MIP and MEP measure the level of pressure a person can generate during inhalation and exhalation, respectively, and are sensitive measures of respiratory muscle strength. MIP is a measure of diaphragm muscle weakness.

In some embodiments, MEP may decline before changes in other pulmonary function tests, including MIP and FVC. In certain embodiments, MEP may be an early indicator of respiratory dysfunction. In certain embodiments, FVC may be used to measure the total volume of air expelled during forced exhalation after maximum inspiration. In patients with DMD, FVC increases concomitantly with physical growth until the early teens. However, as growth slows or is stunted by disease progression, and muscle weakness progresses, the vital capacity enters a descending phase and declines at an average rate of about 8 to 8.5 percent per year after 10 to 12 years of age. In certain embodiments, MIP percent predicted (MIP adjusted for weight), MEP percent predicted (MEP adjusted for age) and FVC percent predicted (FVC adjusted for age and height) are supportive analyses.

As used herein, “sufficient length” refers to an antisense oligonucleotide that is complementary to at least 8, more typically 8-30, contiguous nucleobases in a target dystrophin pre-mRNA. In some embodiments, an antisense of sufficient length includes at least 8, 9, 10, 11, 12, 13, 14, or 15 contiguous nucleobases in the target dystrophin pre-mRNA. In other embodiments an antisense of sufficient length includes at least 16, 17, 18, 19, 20, 21, 22, 23, 24, 25, 26, 27, 28, 29, or 30 contiguous nucleobases in the target dystrophin pre-mRNA. In various embodiments, an oligonucleotide of sufficient length is from about 10 to about 50 nucleotides in length, including oligonucleotides of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 and 40 or more nucleotides. In some embodiments, an oligonucleotide of sufficient length is from 10 to about 30 nucleotides in length. In various embodiments, an oligonucleotide of sufficient length is from 15 to about 25 nucleotides in length. In certain embodiments, an oligonucleotide of sufficient length is from 20 to 30, or 20 to 50, nucleotides in length. In various embodiments, an oligonucleotide of sufficient length is from 25 to 28 nucleotides in length.

The terms “mismatch” or “mismatches” refer to one or more nucleobases (whether contiguous or separate) in an oligomer nucleobase sequence that are not matched to a target pre-mRNA according to base pairing rules. While perfect complementarity is often desired, some embodiments can include one or more but preferably 6, 5, 4, 3, 2, or 1 mismatches with respect to the target pre-mRNA. Variations at any location within the oligomer are included. In certain embodiments, antisense oligomers of the disclosure include variations in nucleobase sequence near the termini variations in the interior, and if present are typically within about 6, 5, 4, 3, 2, or 1 subunits of the 5′ and/or 3′ terminus. In certain embodiments, one, two, or three nucleobases can be removed and still provide on-target binding.

By “enhance” or “enhancing,” or “increase” or “increasing,” or “stimulate” or “stimulating,” refers generally to the ability of one or antisense compounds or compositions to produce or cause a greater physiological response (i.e., downstream effects) in a cell or a subject, as compared to the response caused by either no antisense compound or a control compound. A measurable physiological response may include increased expression of a functional form of a dystrophin protein, or increased dystrophin-related biological activity in muscle tissue, among other responses apparent from the understanding in the art and the description herein. Increased muscle function can also be measured, including increases or improvements in muscle function by about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100%. The levels of functional dystrophin can also be measured, including increased dystrophin expression in about 1%, 2%, %, 15%, 16%, 17%, 18%, 19%, 20%, 25%, 30%, 35%, 40%, 45%, 50%, 55%, 60%, 65%, 70%, 75%, 80%, 85%, 90%, 95%, or 100% of muscle. For instance, it has been shown that around 40% of muscle function improvement can occur if there is 25-30% dystrophin (see, e.g., DelloRusso et al, Proc Natl Acad Sci USA 99: 12979-12984, 2002). An “increased” or “enhanced” amount is typically a “statistically significant” amount, and may include an increase that is 1.1, 1.2, 2, 3, 4, 5, 6, 7, 8, 9, 10, 15, 20, 30, 40, 50 or more times (e.g., 500, 1000 times) (including all integers and decimal points in between and above 1), e.g., 1.5, 1.6, 1.7, 1.8, etc.) the amount produced by no antisense compound (the absence of an agent) or a control compound. The term “reduce” or “inhibit” may relate generally to the ability of one or more antisense compounds of the disclosure to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art, and may include reductions in the symptoms or pathology of muscular dystrophy, or reductions in the expression of defective forms of dystrophin, such as the altered forms of dystrophin that are expressed in individuals with DMD or BMD. A “decrease” in a response may be statistically significant as compared to the response produced by no antisense compound or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between. Also included are vector delivery systems that are capable of expressing the oligomeric, dystrophin-targeting sequences of the present disclosure, such as vectors that express a polynucleotide sequence comprising any one or more of the sequences shown as SEQ ID Nos. 1-10 and 20 in Table 3, and variants thereof, as described herein. By “vector” or “nucleic acid construct” is meant a polynucleotide molecule, preferably a DNA molecule derived, for example, from a plasmid, bacteriophage, yeast or virus, into which a polynucleotide can be inserted or cloned. A vector may contain one or more unique restriction sites and can be capable of autonomous replication in a defined host cell including a target cell or tissue or a progenitor cell or tissue thereof, or be integrated with the genome of the defined host such that the cloned sequence is reproducible. Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated.

“Treatment” of an individual (e.g. a mammal, such as a human) or a cell is any type of intervention used in an attempt to alter the natural course of the individual or cell. Treatment includes, but is not limited to, administration of a pharmaceutical composition or combination therapy, and may be performed either prophylactically or subsequent to the initiation of a pathologic event or contact with an etiologic agent. Treatment includes any desirable effect on the symptoms or pathology of a disease or condition associated with the dystrophin protein, as in certain forms of muscular dystrophy, and may include, for example, minimal changes or improvements in one or more measurable markers of the disease or condition being treated. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

In some embodiments, treatment with an antisense oligonucleotide of the disclosure in combination with a non-steroidal anti-inflammatory compound induces or increases novel dystrophin production, delays disease progression, slows or reduces the loss of ambulation, reduces muscle inflammation, reduces muscle damage, improves muscle function, reduces loss of pulmonary function, and/or enhances muscle regeneration, or any combination thereof, that would be expected without treatment. In some embodiments, treatment maintains, delays, or slows disease progression. In some embodiments, treatment maintains ambulation or reduces the loss of ambulation. In some embodiments, treatment maintains pulmonary function or reduces loss of pulmonary function. In some embodiments, treatment maintains or increases a stable walking distance in a patient, as measured by, for example, the 6 Minute Walk Test (6MWT). In some embodiments, treatment maintains, improves, or reduces the time to walk/run 10 meters (i.e., the 10 meter walk/run test). In some embodiments, treatment maintains, improves, or reduces the time to stand from supine (i.e, time to stand test). In some embodiments, treatment maintains, improves, or reduces the time to climb four standard stairs (i.e., the four-stair climb test). In some embodiments, treatment maintains, improves, or reduces muscle inflammation in the patient, as measured by, for example, MRI (e.g., MRI of the leg muscles). In some embodiments, MRI measures a change in the lower leg muscles. In some embodiments, MRI measures T2 and/or fat fraction to identify muscle degeneration. MRI can identify changes in muscle structure and composition caused by inflammation, edema, muscle damage and fat infiltration. In some embodiments, muscle strength is measured by the North Star Ambulatory Assessment. In some embodiments, muscle strength is measured by the pediatric outcomes data collection instrument (PODCI).

In some embodiments, treatment with an antisense oligonucleotide of the disclosure in combination with a non-steroidal anti-inflammatory compound of the disclosure reduces muscle inflammation, reduces muscle damage, improves muscle function, and/or enhances muscle regeneration. For example, treatment may stabilize, maintain, improve, or reduce inflammation in the subject. Treatment may also, for example, stabilize, maintain, improve, or reduce muscle damage in the subject. Treatment may, for example, stabilize, maintain, or improve muscle function in the subject. In addition, for example, treatment may stabilize, maintain, improve, or enhance muscle regeneration in the subject. In some embodiments, treatment maintains, improves, or reduces muscle inflammation in the patient, as measured by, for example, magnetic resonance imaging (MRI) (e.g., MRI of the leg muscles) that would be expected without treatment.

In some embodiments, treatment with an antisense oligonucleotide of the disclosure in combination a non-steroidal anti-inflammatory compound of the disclosure increases novel dystrophin production and slows or reduces the loss of ambulation that would be expected without treatment. For example, treatment may stabilize, maintain, improve or increase walking ability (e.g., stabilization of ambulation) in the subject. In some embodiments, treatment maintains or increases a stable walking distance in a patient, as measured by, for example, the 6 Minute Walk Test (6MWT), described by McDonald, et al. (Muscle Nerve, 2010; 42:966-74, herein incorporated by reference). A change in the 6 Minute Walk Distance (6MWD) may be expressed as an absolute value, a percentage change or a change in the %-predicted value. In some embodiments, treatment maintains or improves a stable walking distance in a 6MWT from a 20% deficit in the subject relative to a healthy peer. The performance of a DMD patient in the 6MWT relative to the typical performance of a healthy peer can be determined by calculating a %-predicted value. For example, the %-predicted 6MWD may be calculated using the following equation for males: 196.72 +(39.81×age)−(1.36×age²)+(132.28×height in meters). For females, the %-predicted 6MWD may be calculated using the following equation: 188.61+(51.50×age)−(1.86×age²)+(86.10×height in meters) (Henricson et al. PLoS Curr., 2012, version 2, herein incorporated by reference). In some embodiments, treatment with an antisense oligonucleotide increases the stable walking distance in the patient from baseline to greater than 3, 5, 6, 7, 8, 9, 10, 15, 20, 25, 30 or 50 meters (including all integers in between).

Loss of muscle function in patients with DMD may occur against the background of normal childhood growth and development. Indeed, younger children with DMD may show an increase in distance walked during 6MWT over the course of about 1 year despite progressive muscular impairment. In some embodiments, the 6MWD from patients with DMD is compared to typically developing control subjects and to existing normative data from age and sex matched subjects. In some embodiments, normal growth and development can be accounted for using an age and height based equation fitted to normative data. Such an equation can be used to convert 6MWD to a percent-predicted (%-predicted) value in subjects with DMD. In certain embodiments, analysis of %-predicted 6MWD data represents a method to account for normal growth and development, and may show that gains in function at early ages (e.g., less than or equal to age 7) represent stable rather than improving abilities in patients with DMD (Henricson et al. PLoS Curr., 2012, version 2, herein incorporated by reference).

“Co-administration” or “co-administering” or “combination therapy” as used herein, generally refers to the administration of a DMD exon-skipping antisense oligonucleotide in combination with one or more non-steroidal anti-inflammatory compounds disclosed herein. In other words, the terms “co-administering” or “co-administration” or “combination therapy” means administration of the DMD exon-skipping antisense oligonucleotide, such as golodirsen, concomitantly in a pharmaceutically acceptable dosage form with one or more non-steroidal anti-inflammatory compounds and optionally one or more glucocorticoids disclosed herein. Each therapeutic agent in a combination therapy disclosed herein may be administered either alone or in a medicament (also referred to herein as a pharmaceutical composition) which comprises the therapeutic agent and one or more pharmaceutically acceptable carriers, excipients and diluents, according to standard pharmaceutical practice. Each therapeutic agent may be prepared by formulating a compound or pharmaceutically acceptable salt thereof separately, and the both may be administered either at the same time or separately. Further, the two formulations may be placed in a single package, to provide the so called kit formulation. In some configurations, both compounds may be contained in a single formulation.

Each therapeutic agent in a combination therapy disclosed herein may be administered simultaneously (i.e., in the same medicament), concurrently (i.e., in separate medicaments administered one right after the other in any order) or sequentially in any order. Sequential administration is particularly useful when the therapeutic agents in the combination therapy are in different dosage forms (one agent is a tablet or capsule and another agent is a sterile liquid) and/or are administered on different dosing schedules, e.g., tablet or capsule formulated for daily administration and a composition formulated for parenteral administration, such as once weekly, once every two weeks, or once every three weeks.

In some embodiments, the terms “co-administering” or “co-administration” or “combination therapy” mean the administration of the DMD exon-skipping antisense oligonucleotide, such as golodirsen, concomitantly in a pharmaceutically acceptable dosage form with one or more non-steroidal anti-inflammatory compounds and optionally one or more glucocorticoids disclosed herein: (i) in the same dosage form, e.g., the same tablet or pharmaceutical composition, meaning a pharmaceutical composition comprising a DMD exon-skipping antisense oligonucleotide, such as golodirsen, one or more non-steroidal anti-inflammatory compounds disclosed herein, and optionally one or more glucocorticoids and a pharmaceutically acceptable carrier; (ii) in a separate dosage form having the same mode of administration, e.g., a kit comprising a first pharmaceutical composition suitable for parenteral administration comprising a DMD exon-skipping antisense oligonucleotide, such as golodirsen, and a pharmaceutically acceptable carrier, a second pharmaceutical composition suitable for parenteral administration comprising one or more non-steroidal anti-inflammatory compounds disclosed herein and a pharmaceutically acceptable carrier, and optionally a third pharmaceutical composition suitable for parenteral administration comprising one or more glucocorticoids disclosed herein and a pharmaceutically acceptable carrier; and (iii) in a separate dosage form having different modes of administration, e.g., a kit comprising a first pharmaceutical composition suitable for parenteral administration comprising a DMD exon-skipping antisense oligonucleotide, such as golodirsen, and a pharmaceutically acceptable carrier, a second pharmaceutical composition suitable for oral administration comprising one or more non-steroidal anti-inflammatory compounds disclosed herein and a pharmaceutically acceptable carrier, and optionally a third pharmaceutical composition suitable for oral administration comprising one or more glucocorticoids disclosed herein and a pharmaceutically acceptable carrier.

Further, those of skill in the art given the benefit of the present disclosure will appreciate that when more than one non-steroidal anti-inflammatory compound disclosed herein is being administered, the agents need not share the same mode of administration, e.g., a kit comprising a first pharmaceutical composition suitable for parenteral administration comprising a DMD exon-skipping antisense oligonucleotide, such as golodirsen, and a pharmaceutically acceptable carrier, a second pharmaceutical composition suitable for oral administration comprising a first non-steroidal anti-inflammatory compound disclosed herein and a pharmaceutically acceptable carrier. Those of skill in the art will appreciate that the concomitant administration referred to above in the context of “co-administering” or “co-administration” means that the pharmaceutical composition comprising DMD exon-skipping antisense oligonucleotide and a pharmaceutical composition(s) comprising the non-steroidal anti-inflammatory compound can be administered on the same schedule, i.e., at the same time and day, or on a different schedule, i.e., on different, although not necessarily distinct, schedules.

In that regard, when the pharmaceutical composition comprising a DMD exon-skipping antisense oligonucleotide and a pharmaceutical composition(s) comprising the non-steroidal anti-inflammatory compound is administered on a different schedule, such a different schedule may also be referred to herein as “background” or “background administration.” For example, the pharmaceutical composition comprising a DMD exon-skipping antisense oligonucleotide may be administered in a certain dosage form twice a day, and the pharmaceutical composition(s) comprising the non-steroidal anti-inflammatory compound may be administered once a day, such that the pharmaceutical composition comprising the DMD exon-skipping antisense oligonucleotide may but not necessarily be administered at the same time as the pharmaceutical composition(s) comprising the non-steroidal anti-inflammatory compound during one of the daily administrations. Other suitable variations to “co-administering”, “co-administration” or “combination therapy” will be readily apparent to those of skill in the art given the benefit of the present disclosure and are part of the meaning of this term.

“Chronic administration,” as used herein, refers to continuous, regular, long-term administration, Le., periodic administration without substantial interruption. For example, daily, for a period of time of at least several weeks or months or years, for the purpose of treating muscular dystrophy in a patient. For example, weekly, for a period of time of at least several months or years, for the purpose of treating muscular dystrophy in a patient (e,g., weekly for at least six weeks, weekly for at least 12 weeks, weekly for at least 24 weeks, weekly for at least 48 weeks, weekly for at least 72 weeks, weekly for at least 96 weeks, weekly for at least 120 weeks, weekly for at least 144 weeks, weekly for at least 168 weeks, weekly for at least 180 weeks, weekly for at least 192 weeks, weekly for at least 216 weeks, or weekly for at least 240 weeks).

“Periodic administration,” as used herein, refers to administration with an interval between doses. For example, periodic administration includes administration at fixed intervals (e.g., weekly, monthly) that may be recurring,

“Placebo,” as used herein, refers to a substance that has no effect and may be used as a control.

“Placebo control,” as used herein, refers to a subject or patient that receives a placebo rather than the combination therapy, antisense oligonucleotide, non-steroidal anti-inflammatory compound, and/or another pharmaceutical composition. The placebo control may have the same mutation status, be of similar age, similar ability to ambulate, and or receive the same concomitant medications (including steroids, etc.), as the subject or patient.

A “subject,” or “patient” as used herein, includes any animal that exhibits a symptom, or is at risk for exhibiting a symptom, which can be treated with an antisense compound of the disclosure, such as a subject that has or is at risk for having DMD or BMD, or any of the symptoms associated with these conditions (e.g., muscle fibre loss). Suitable subjects (patients) include laboratory animals (such as mouse, rat, rabbit, or guinea pig), farm animals, and domestic animals or pets (such as a cat or dog). Non-human primates and, in some embodiments, human patients, are included.

A “pediatric patient” as used herein is a patient from age 1 to 21, inclusive.

An antisense molecule nomenclature system was proposed and published to distinguish between the different antisense molecules (see Mann et al., (2002) J Gen Med 4, 644-654). This nomenclature became especially relevant when testing several slightly different antisense molecules, all directed at the same target region, as shown below:

### H#A/D(x:y).

The first letter designates the species (e.g. H: human, M: murine, C: canine). “#” designates target dystrophin exon number. “A/D” indicates acceptor or donor splice site at the beginning and end of the exon, respectively. (x y) represents the annealing coordinates where “−” or “+” indicate intronic or exonic sequences respectively. For example, A(−6+18) would indicate the last 6 bases of the intron preceding the target exon and the first 18 bases of the target exon. The closest splice site would be the acceptor so these coordinates would be preceded with an “A”. Describing annealing coordinates at the donor splice site could be D(+2−18) where the last 2 exonic bases and the first 18 intronic bases correspond to the annealing site of the antisense molecule. Entirely exonic annealing coordinates that would be represented by A(+65+85), that is the site between the 65th and 85th nucleotide from the start of that exon.

B. Antisense Oligonucleotides and Uses Thereof

Antisense oligonucleotides that target the pre-mRNA of the dystrophin gene to effect the skipping of exon 53 are used accordance with the methods of this disclosure.

Such an antisense oligomer can be designed to block or inhibit translation of mRNA or to inhibit natural pre-mRNA splice processing, and may be said to be “directed to” or “targeted against” a target sequence with which it hybridizes. The target sequence is typically a region including an AUG start codon of an mRNA, a Translation Suppressing Oligomer, or splice site of a pre-processed mRNA, a Splice Suppressing Oligomer (SSO). The target sequence for a splice site may include an mRNA sequence having its 5′ end 1 to about 25 base pairs downstream of a normal splice acceptor junction in a preprocessed mRNA. In some embodiments, a target sequence may be any region of a preprocessed mRNA that includes a splice site or is contained entirely within an exon coding sequence or spans a splice acceptor or donor site. An oligomer is more generally said to be “targeted against” a biologically relevant target, such as a protein, virus, or bacteria, when it is targeted against the nucleic acid of the target in the manner described above.

In certain embodiments, the antisense oligonucleotide specifically hybridizes to a target region of exon 53 of the human dystrophin pre-mRNA and induces exon 53 skipping. For example, the antisense oligonucleotide is golodirsen.

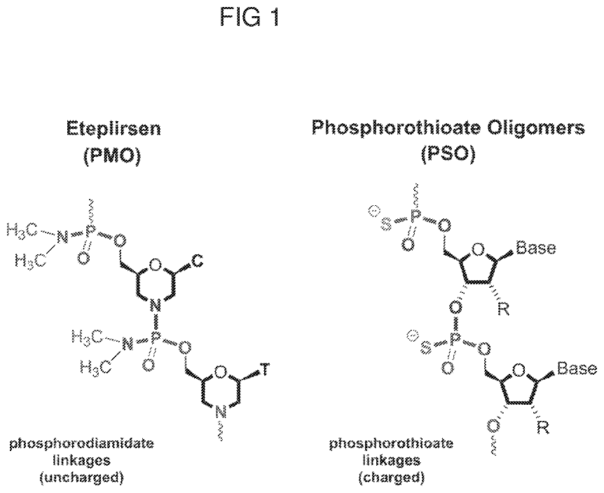

Golodirsen belongs to a distinct class of novel synthetic antisense RNA therapeutics called Phosphorodiamidate Morpholino Oligomers (PMO), which are a redesign of the natural nucleic acid structure (FIG. 1). Golodirsen is a PMO that hybridizes to an exon 53 target region of the Dystrophin pre-mRNA and induces exon 53 skipping. PMOs offer potential clinical advantages based on in vivo nonclinical observations.

PMOs incorporate modifications to the sugar ring of RNA that protect it from enzymatic degradation by nucleases in order to ensure stability in vivo. PMOs are distinguished from natural nucleic acids and other antisense oligonucleotide classes in part through the use of 6-membered synthetic morpholino rings, which replace the 5-membered ribofuranosyl rings found in RNA, DNA and many other synthetic antisense RNA oligonucleotides.

The uncharged phosphorodiamidate linkages specific to PMOs are considered to potentially confer reduced off-target binding to proteins. PMOs have an uncharged phosphorodiamidate linkage that links each morpholino ring instead of the negatively charged phosphorothioate linkage used in other clinical-stage synthetic antisense RNA oligonucleotides.

A potential approach to the treatment of DMD caused by out-of-frame mutations in the DMD gene is suggested by the milder form of dystrophinopathy known as BMD, which is caused by in-frame mutations. The ability to convert an out-of-frame mutation to an in-frame mutation would hypothetically preserve the mRNA reading frame and produce an internally shortened yet functional dystrophin protein. Golodirsen was designed to accomplish this.

Golodirsen targets dystrophin pre-mRNA and induces skipping of exon 53, so it is excluded or skipped from the mature, spliced mRNA transcript. By skipping exon 53, the disrupted reading frame is restored to an in-frame mutation. While DMD is comprised of various genetic subtypes, golodirsen was specifically designed to skip exon 53 of dystrophin pre-mRNA. DMD mutations amenable to skipping exon 53 include deletions of exons contiguous to exon 53 (i.e. including deletion of exon 52 or exon 54), and comprise a subgroup of DMD patients (8%).

The sequence of golodirsen's 25 nucleobases is designed to be complementary to a specific target region at (+36+60) within exon 53 of dystrophin pre-mRNA. Each morpholino ring in golodirsen is linked to one of four heterocyclic nucleobases found in DNA (adenine, cytosine, guanine, and thymine).

Hybridization of golodirsen with the targeted pre-mRNA sequence interferes with formation of the pre-mRNA splicing complex and deletes exon 53 from the mature mRNA. The structure and conformation of golodirsen allows for sequence-specific base pairing to the complementary sequence. For example, eteplirsen, which is a PMO that was designed to skip exon 51 of dystrophin pre-mRNA allows for sequence-specific base pairing to the complementary sequence contained in exon 51 of dystrophin pre-mRNA.

In certain embodiments, an antisense oligomer conjugate of the disclosure is according to the Formula:

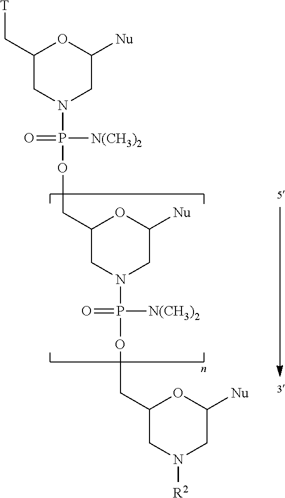

wherein:

each Nu is a nucleobase which taken together form a targeting sequence; and

T is a moiety selected from:

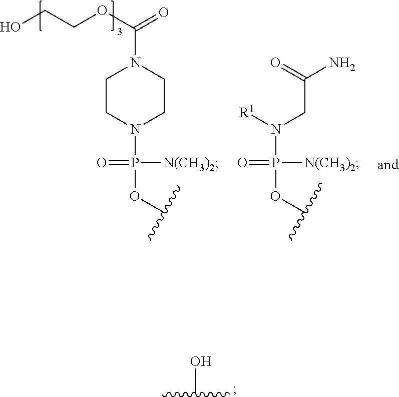

and

R¹ is Cₗ-C₆ alkyl, R² is selected from H, acetyl or a cell penetrating peptide comprising a sequence selected from one of SEQ ID NO:11-19 and n is from 16 to 28;

wherein the targeting sequence is selected from one of SEQ ID NO:1-10 and 20; and an effective amount of a non-steroidal anti-inflammatory compound, thereby treating the patient with DMD. In one aspect, R² is a cell penetrating peptide consisting of SEQ ID NO: 19. In one aspect, n is 23 and the targeting sequence is SEQ ID NO: 1.

C. Oligomer Chemistry Features

The antisense oligomers of the disclosure can employ a variety of antisense oligomer chemistries. Examples of oligomer chemistries include, without limitation, morpholino oligomers, phosphorothioate modified oligomers, 2′-O-methyl modified oligomers, peptide nucleic acid (PNA), locked nucleic acid (LNA), phosphorothioate oligomers, 2′-O-MOE modified oligomers, 2′-fluoro-modified oligomers, 2′-O,4′C-ethylene-bridged nucleic acids (ENAs), tricyclo-DNAs, tricyclo-DNA phosphorothioate subunits, 2′-O-[2-(N-methylcarbamoyl)ethyl]modified oligomers, including combinations of any of the foregoing. Phosphorothioate and 2′-O-Me-modified chemistries can be combined to generate a 2′-O-Me-phosphorothioate backbone. See, e.g., PCT Publication Nos. WO/2013/112053 and WO/2009/008725, which are hereby incorporated by reference in their entireties. Exemplary embodiments of oligomer chemistries of the disclosure are further described below.

1. Peptide Nucleic Acids (PNAs)

Peptide nucleic acids (PNAs) are analogs of DNA in which the backbone is structurally homomorphous with a deoxyribose backbone, consisting of N-(2-aminoethyl) glycine units to which pyrimidine or purine bases are attached. PNAs containing natural pyrimidine and purine bases hybridize to complementary oligomers obeying Watson-Crick base-pairing rules, and mimic DNA in terms of base pair recognition . The backbone of PNAs is formed by peptide bonds rather than phosphodiester bonds, making them well-suited for antisense applications (see structure below). The backbone is uncharged, resulting in PNA/DNA or PNA/RNA duplexes that exhibit greater than normal thermal stability. PNAs are not recognized by nucleases or proteases. A non-limiting example of a PNA is depicted below.

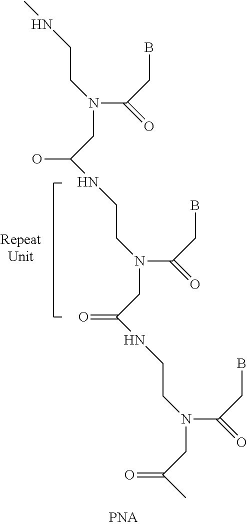

Despite a radical structural change to the natural structure, PNAs are capable of sequence-specific binding in a helix form to DNA or RNA. Characteristics of PNAs include a high binding affinity to complementary DNA or RNA, a destabilizing effect caused by single-base mismatch, resistance to nucleases and proteases, hybridization with DNA or RNA independent of salt concentration and triplex formation with homopurine DNA. PANAGENE™ has developed its proprietary Bts PNA monomers (Bts; benzothiazole-2-sulfonyl group) and proprietary oligomerization process. The PNA oligomerization using Bts PNA monomers is composed of repetitive cycles of deprotection, coupling and capping. PNAs can be produced synthetically using any technique known in the art. See, e.g., U .S. Pat. Nos.: 6,969,766; 7,211,668; 7,022,851; 7,125,994; 7,145,006; and 7,179,896. See also U.S. Pat. Nos.: 5,539,082; 5,714,331; and 5,719,262 for the preparation of PNAs. Further teaching of PNA compounds can be found in Nielsen et al., Science, 254:1497-1500, 1991. Each of the foregoing is incorporated by reference in its entirety.

In certain embodiments, the antisense oligonucleotides of SEQ ID Nos: 1-10and 20 in Table 3 may be PNA oligomers. In certain embodiments, the antisense oligonucleotide of SEQ ID NO: 1 is a PNA oligomer.

2. Locked Nucleic Acids (LNAs)

Antisense oligomers may also contain “locked nucleic acid” subunits (LNAs). “LNAs” are a member of a class of modifications called bridged nucleic acid (BNA). BNA is characterized by a covalent linkage that locks the conformation of the ribose ring in a C30-endo (northern) sugar pucker. For LNA, the bridge is composed of a methylene between the 2′-O and the 4′-C positions. LNA enhances backbone preorganization and base stacking to increase hybridization and thermal stability.

The structures of LNAs can be found, for example, in Wengel, et al., Chemical Communications (1998) 455; Koshkin et al., Tetrahedron (1998) 54:3607; Jesper Wengel, Accounts of Chem. Research (1999) 32:301; Obika, et al., Tetrahedron Letters (1997) 38:8735; Obika, et al., Tetrahedron Letters (1998) 39:5401; and Obika, et al., Bioorganic Medicinal Chemistry (2008) 16:9230, which are hereby incorporated by reference in their entirety. A non-limiting example of an LNA is depicted below.

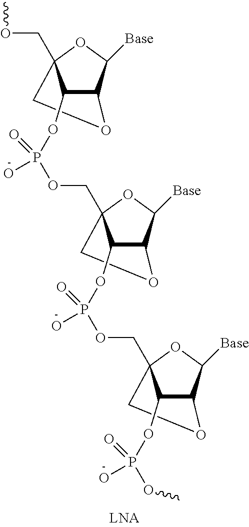

Antisense oligomers of the disclosure may incorporate one or more LNAs; in some cases, the antisense oligomers may be entirely composed of LNAs. Methods for the synthesis of individual LNA nucleoside subunits and their incorporation into oligomers are described, for example, in U.S. Pat. Nos. 7,572,582; 7,569,575; 7,084,125; 7,060,809; 7,053,207; 7,034,133; 6,794,499; and 6,670,461; each of which is incorporated by reference in its entirety. Typical intersubunit linkers include phosphodiester and phosphorothioate moieties; alternatively, non-phosphorous containing linkers may be employed. Further embodiments include an LNA containing antisense oligomer where each LNA subunit is separated by a DNA subunit. Certain antisense oligomers are composed of alternating LNA and DNA subunits where the intersubunit linker is phosphorothioate.

2′O,4′C-ethylene-bridged nucleic acids (ENAs) are another member of the class of BNAs. A non-limiting example is depicted below.

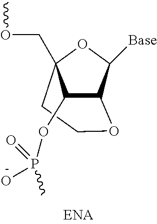

ENA oligomers and their preparation are described in Obika et al., Tetrahedron Lett (1997) 38 (50): 8735, which is hereby incorporated by reference in its entirety. Antisense oligomers of the disclosure may incorporate one or more ENA subunits.

In certain embodiments, the antisense oligonucleotides of SEQ ID Nos: 1-10 and 20 in Table 3 may be LNA oligomers. In certain embodiments, the antisense oligonucleotide of SEQ ID NO: 1 is a LNA oligomer. In certain embodiments, the antisense oligonucleotides of SEQ ID Nos: 1-10 and 20 in Table 3 may be BNA oligomers. In certain embodiments, the antisense oligonucleotide of SEQ ID NO: 1 is a BNA oligomer. In certain embodiments, the antisense oligonucleotides of SEQ ID Nos: 1-10 and 20 in Table 3 may be ENA oligomers. In certain embodiments, the antisense oligonucleotide of SEQ ID NO: 1 is an ENA oligomer.

3. Unlocked Nucleic Acid (UNA)

Antisense oligomers may also contain unlocked nucleic acid (UNA) subunits. UNAs and UNA oligomers are an analogue of RNA in which the C2′-C3′ bond of the subunit has been cleaved. Whereas LNA is conformationally restricted (relative to DNA and RNA), UNA is very flexible. UNAs are disclosed, for example, in WO 2016/070166. A non-limiting example of an UNA is depicted below.

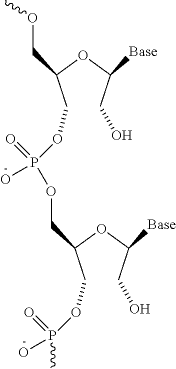

Typical intersubunit linkers include phosphodiester and phosphorothioate moieties; alternatively, non-phosphorous containing linkers may be employed.

In certain embodiments, the antisense oligonucleotides of SEQ ID Nos: 1-10 and 20 in Table 3 may be UNA oligomers. In certain embodiments, the antisense oligonucleotide of SEQ ID NO: 1 is a UNA oligomer.

4. Phosphorothioates

“Phosphorothioates” (or S-oligos) are a variant of normal DNA in which one of the nonbridging oxygens is replaced by a sulfur. A non-limiting example of a phosphorothioate is depicted below.

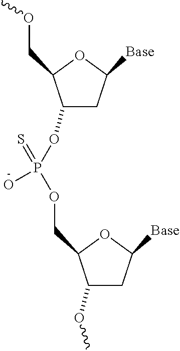

The sulfurization of the internucleotide bond reduces the action of endo-and exonucleases including 5′ to 3′ and 3′ to 5′ DNA POL 1 exonuclease, nucleases S1 and P1, RNases, serum nucleases and snake venom phosphodiesterase. Phosphorothioates are made by two principal routes: by the action of a solution of elemental sulfur in carbon disulfide on a hydrogen phosphonate, or by the method of sulfurizing phosphite triesters with either tetraethylthiuram disulfide (TETD) or 3H-1,2-benzodithiol-3-one 1,1-dioxide (BDTD) (see, e.g., Iyer et al., J. Org. Chem. 55, 4693-4699, 1990, which is hereby incorporated by reference in its entirety). The latter methods avoid the problem of elemental sulfur's insolubility in most organic solvents and the toxicity of carbon disulfide. The TETD and BDTD methods also yield higher purity phosphorothioates.

In certain embodiments, the antisense oligonucleotides of SEQ ID Nos: 1-10 and 20 in Table 3 may be phosphorothioate oligomers. In certain embodiments, the antisense oligonucleotide of SEQ ID NO: 1 is a phosphorothioate oligomer.

5. Triclyclo-DNAs and Tricyclo-Phosphorothioate Subunits

Tricyclo-DNAs (tc-DNA) are a class of constrained DNA analogs in which each nucleotide is modified by the introduction of a cyclopropane ring to restrict conformational flexibility of the backbone and to optimize the backbone geometry of the torsion angle γ. Homobasic adenine- and thymine-containing tc-DNAs form extraordinarily stable A-T base pairs with complementary RNAs. Tricyclo-DNAs and their synthesis are described in International Patent Application Publication No. WO 2010/115993, which is hereby incorporated by reference in its entirety. Antisense oligomers of the disclosure may incorporate one or more tricycle-DNA subunits; in some cases, the antisense oligomers may be entirely composed of tricycle-DNA subunits.

Tricyclo-phosphorothioate subunits are tricyclo-DNA subunits with phosphorothioate intersubunit linkages. Tricyclo-phosphorothioate subunits and their synthesis are described in International Patent Application Publication No. WO 2013/053928, which is hereby incorporated by reference in its entirety. Antisense oligomers of the disclosure may incorporate one or more tricycle-DNA subunits; in some cases, the antisense oligomers may be entirely composed of tricycle-DNA subunits. A non-limiting example of a tricycle-DNA/tricycle- phosphorothioate subunit is depicted below.

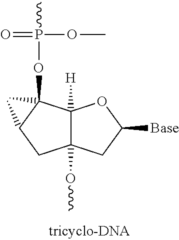

In certain embodiments, the antisense oligonucleotides of SEQ ID Nos: 1-10 and 20 in Table 3 may be tricyclo-phosphorothioate oligomers. In certain embodiments, the antisense oligonucleotide of SEQ ID NO: 1 is a tricylco-phosphorothioate oligomer.

6. 2′-O-Methyl, 2′-O-MOE, and 2′-F Oligomers “2′-O-Me oligomer” molecules carry a methyl group at the 2′-OH residue of the ribose molecule. 2′-O-Me-RNAs show the same (or similar) behavior as DNA, but are protected against nuclease degradation. 2′-O-Me-RNAs can also be combined with phosphorothioate oligomers (PTOs) for further stabilization. 2′O-Me oligomers (phosphodiester or phosphorothioate) can be synthesized according to routine techniques in the art (see, e.g., Yoo et al., Nucleic Acids Res. 32:2008-16, 2004, which is hereby incorporated by reference in its entirety). A non-limiting example of a 2′-O-Me oligomer is depicted below.

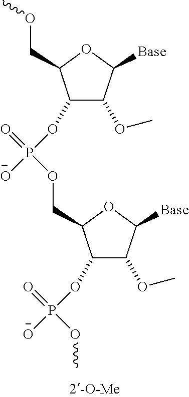

2′-O-Methoxyethyl Oligomers (2′-O-MOE) carry a methoxyethyl group at the 2′-OH residue of the ribose molecule and are discussed in Martin et al., Helv. Chim. Acta, 78, 486-504, 1995, which is hereby incorporated by reference in its entirety. A non-limiting example of a 2′-O-MOE subunit is depicted below.

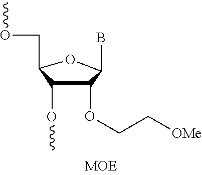

2′-Fluoro (2′-F) oligomers have a fluoro radical in at the 2′ position in place of the 2′-OH. A non-limiting example of a 2′-F oligomer is depicted below.

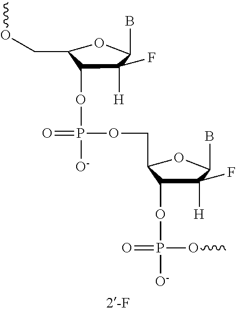

2′-fluoro oligomers are further described in WO 2004/043977, which is hereby incorporated by reference in its entirety.

2′-O-Methyl, 2′-O-MOE, and 2′-F oligomers may also comprise one or more phosphorothioate (PS) linkages as depicted below.

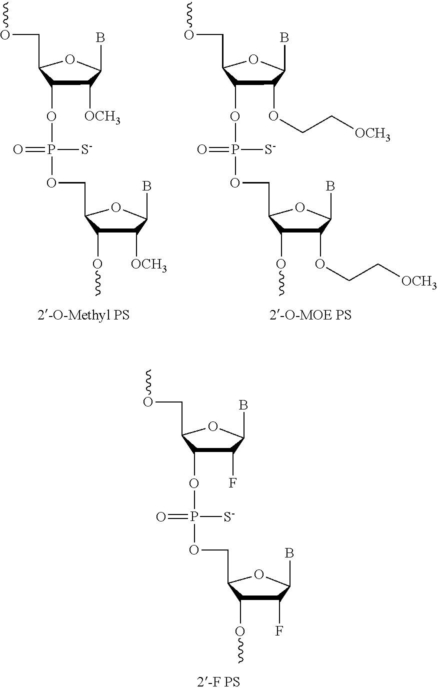

Additionally, 2′-O-Methyl, 2′-O-MOE, and 2′-F oligomers may comprise PS intersubunit linkages throughout the oligomer, for example, as in'the 2′-O-methyl PS oligomer drisapersen depicted below.

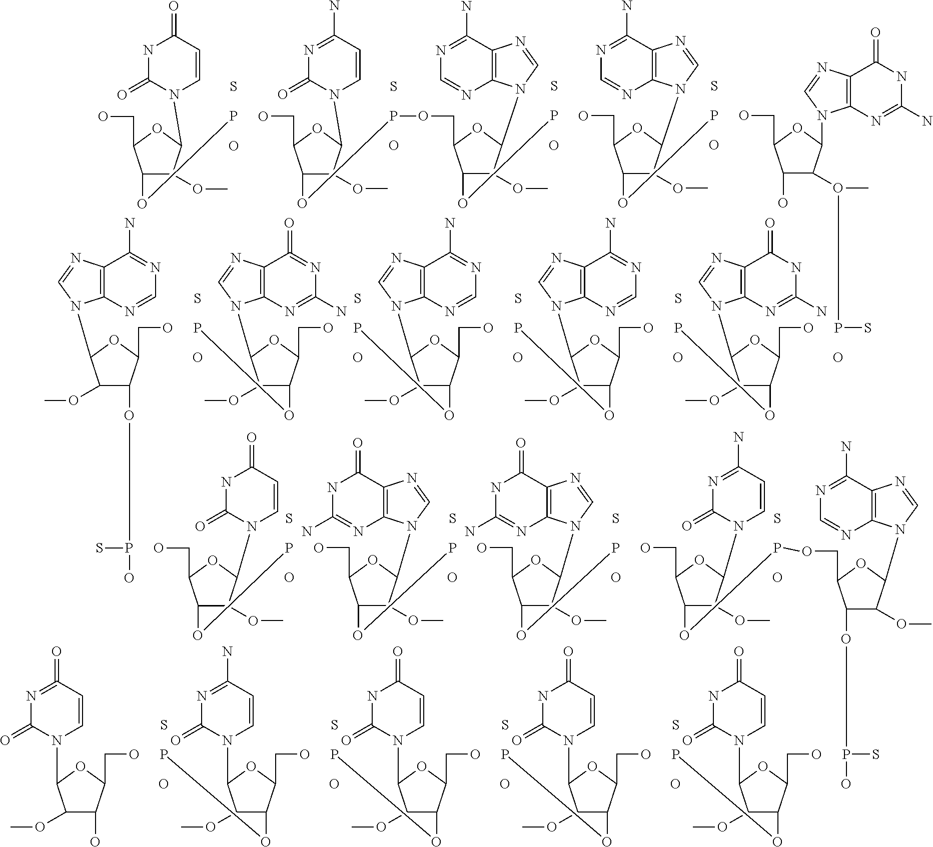

Alternatively, 2′-O-Methyl, 2′-O-MOE, and/or 2′-F oligomers may comprise PS linkages at the ends of the oligomer, as depicted below.

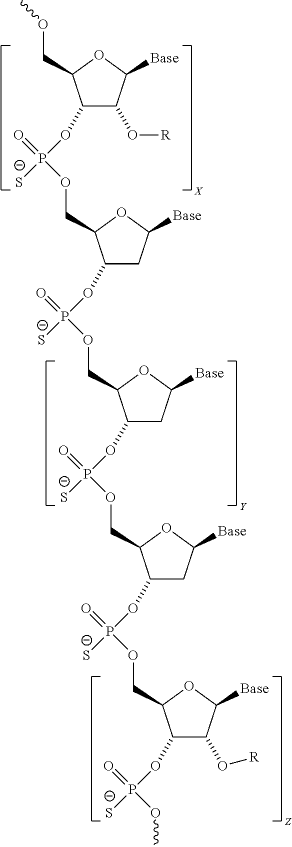

where:

R is CH₂CH₂OCH₃ (methoxyethyl or MOE); and

X, Y, and Z denote the number of nucleotides contained within each of the designated 5′-wing, central gap, and 3′-wing regions, respectively.

Antisense oligomers of the disclosure may incorporate one or more 2′-O-Methyl, 2′-O-MOE, and 2′-F subunits and may utilize any of the intersubunit linkages described here. In some instances, an antisense oligomer of the disclosure may be composed of entirely 2′-O-Methyl, 2′-O-MOE, or 2′-F subunits. One embodiment of an antisense oligomers of the disclosure is composed entirely of 2′-O-methyl subunits.

In certain embodiments, the antisense oligonucleotides of SEQ ID Nos: 1-10 and 20 in Table 3 may be 2′-O-Me oligomers. In certain embodiments, the antisense oligonucleotide of SEQ ID NO: 1 is a 2′-O-Me oligomer. In certain embodiments, the antisense oligonucleotides of SEQ ID Nos: 1-10 and 20 in Table 3 may be 2′-O-Methoxyethyl oligomers. In certain embodiments, the antisense oligonucleotide of SEQ ID NO: 1 is a 2′-O-Methoxyethyl oligomer. In certain embodiments, the antisense oligonucleotides of SEQ ID Nos: 1-10 and 20 in Table 3 may be 2′-Fluoro oligomers. In certain embodiments, the antisense oligonucleotide of SEQ ID NO: 1 is a 2′-Fluoro oligomer.

7. 2′-O-[2-(N-methylcarbamoyl)ethyl] Oligomers (MCEs)

MCEs are another example of 2′-O modified ribonucleosides useful in the antisense oligomers of the disclosure. Here, the 2′-OH is derivatized to a 2-(N-methylcarbamoyl)ethyl moiety to increase nuclease resistance. A non-limiting example of an MCE oligomer is depicted below.

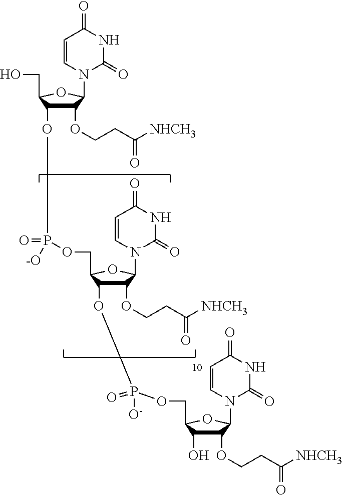

MCEs and their synthesis are described in Yamada et al., J. Org. Chem. (2011) 76(9):3042-53, which is hereby incorporated by reference in its entirety. Antisense oligomers of the disclosure may incorporate one or more MCE subunits.

In certain embodiments, the antisense oligonucleotides of SEQ ID Nos: 1-10 and 20 in Table 3 may be MCE oligomers. In certain embodiments, the antisense oligonucleotide of SEQ ID NO: 1 is a MCE oligomer.

8. Stereo Specific Oligomers

Stereo specific oligomers are those in which the stereo chemistry of each phosphorous-containing linkage is fixed by the method of synthesis such that a substantially stereo-pure oligomer is produced. A non-limiting example of a stereo specific oligomer is depicted below.

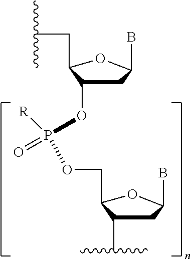

In the above example, each phosphorous of the oligomer has the same stereo configuration. Additional examples include the oligomers described herein. For example, LNAs, ENAs, Tricyclo-DNAs, MCEs, 2′-O-Methyl, 2′-O-MOE, 2′-F, and morpholino-based oligomers can be prepared with stereo-specific phosphorous-containing internucleoside linkages such as, for example, phosphorothioate, phosphodiester, phosphoramidate, phosphorodiamidate, or other phosphorous-containing internucleoside linkages. Stereo specific oligomers, methods of preparation, chiral controlled synthesis, chiral design, and chiral auxiliaries for use in preparation of such oligomers are detailed, for example, in WO2017192664, WO2017192679, WO2017062862, WO2017015575, WO2017015555, WO2015107425, WO2015108048, WO2015108046, WO2015108047, WO2012039448, WO2010064146, WO2011034072, WO2014010250, WO2014012081, WO20130127858, and WO2011005761, each of which is hereby incorporated by reference in its entirety.

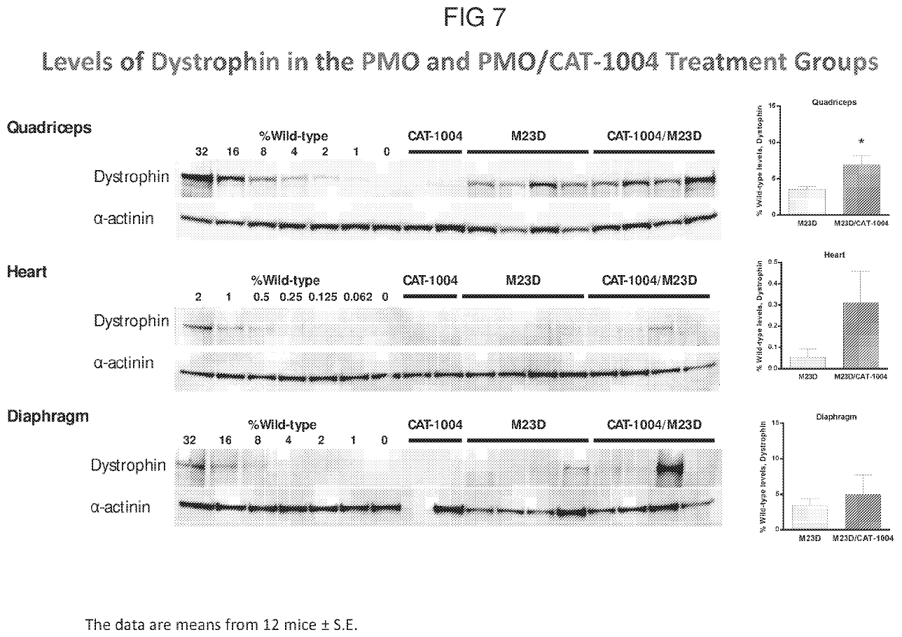

Stereo specific oligomers can have phosphorous-containing internucleoside linkages in an Rₚ or Sₚ configuration. Chiral phosphorous-containing linkages in which the stereo configuration of the linkages is controlled is referred to as “stereopure,” while chiral phosphorous-containing linkages in which the stereo configuration of the linkages is uncontrolled is referred to as “stereorandom.” In certain embodiments, the oligomers of the disclosure comprise a plurality of stereopure and stereorandom linkages, such that the resulting oligomer has stereopure subunits at pre-specified positions of the oligomer. An example of the location of the stereopure subunits is provided in international patent application publication number WO 2017/062862 A2 in FIGS. 7A and 7B. In an embodiment, all the chiral phosphorous-containing linkages in an oligomer are stereorandom. In an embodiment, all the chiral phosphorous-containing linkages in an oligomer are stereopure.

In an embodiment of an oligomer with n chiral phosphorous-containing linkages (where n is an integer of 1 or greater), all n of the chiral phosphorous-containing linkages in the oligomer are stereorandom. In an embodiment of an oligomer with n chiral phosphorous-containing linkages (where n is an integer of 1 or greater), all n of the chiral phosphorous-containing linkages in the oligomer are stereopure. In an embodiment of an oligomer with n chiral phosphorous-containing linkages (where n is an integer of 1 or greater), at least 10% (to the nearest integer) of the n phosphorous-containing linkages in the oligomer are stereopure. In an embodiment of an oligomer with n chiral phosphorous-containing linkages (where n is an integer of 1 or greater), at least 20% (to the nearest integer) of the n phosphorous-containing linkages in the oligomer are stereopure. In an embodiment of an oligomer with n chiral phosphorous-containing linkages (where n is an integer of 1 or greater), at least 30% (to the nearest integer) of the n phosphorous-containing linkages in the oligomer are stereopure. In an embodiment of an oligomer with n chiral phosphorous-containing linkages (where n is an integer of 1 or greater), at least 40% (to the nearest integer) of the n phosphorous-containing linkages in the oligomer are stereopure. In an embodiment of an oligomer with n chiral phosphorous-containing linkages (where n is an integer of 1 or greater), at least 50% (to the nearest integer) of the n phosphorous-containing linkages in the oligomer are stereopure. In an embodiment of an oligomer with n chiral phosphorous-containing linkages (where n is an integer of 1 or greater), at least 60% (to the nearest integer) of the n phosphorous-containing linkages in the oligomer are stereopure. In an embodiment of an oligomer with n chiral phosphorous-containing linkages (where n is an integer of 1 or greater), at least 70% (to the nearest integer) of the n phosphorous-containing linkages in the oligomer are stereopure. In an embodiment of an oligomer with n chiral phosphorous-containing linkages (where n is an integer of 1 or greater), at least 80% (to the nearest integer) of the n phosphorous-containing linkages in the oligomer are stereopure. In an embodiment of an oligomer with n chiral phosphorous-containing linkages (where n is an integer of 1 or greater), at least 90% (to the nearest integer) of the n phosphorous-containing linkages in the oligomer are stereopure.

In an embodiment of an oligomer with n chiral phosphorous-containing linkages (where n is an integer of 1 or greater), the oligomer contains at least 2 contiguous stereopure phosphorous-containing linkages of the same stereo orientation (i.e. either Sₚ or Rₚ). In an embodiment of an oligomer with n chiral phosphorous-containing linkages (where n is an integer of 1 or greater), the oligomer contains at least 3 contiguous stereopure phosphorous-containing linkages of the same stereo orientation (i.e. either Sₚ or Rₚ). In an embodiment of an oligomer with n chiral phosphorous-containing linkages (where n is an integer of 1 or greater), the oligomer contains at least 4 contiguous stereopure phosphorous-containing linkages of the same stereo orientation (i.e. either Sₚ or Rₚ). In an embodiment of an oligomer with n chiral phosphorous-containing linkages (where n is an integer of 1 or greater), the oligomer contains at least 5 contiguous stereopure phosphorous-containing linkages of the same stereo orientation (i.e. either Sₚ or Rₚ). In an embodiment of an oligomer with n chiral phosphorous-containing linkages (where n is an integer of 1 or greater), the oligomer contains at least 6 contiguous stereopure phosphorous-containing linkages of the same stereo orientation (i.e. either Sₚ or Rₚ). In an embodiment of an oligomer with n chiral phosphorous-containing linkages (where n is an integer of 1 or greater), the oligomer contains at least 7 contiguous stereopure phosphorous-containing linkages of the same stereo orientation (i.e. either Sₚ or Rₚ). In an embodiment of an oligomer with n chiral phosphorous-containing linkages (where n is an integer of 1 or greater), the oligomer contains at least 8 contiguous stereopure phosphorous-containing linkages of the same stereo orientation (i.e. either Sₚ or Rₚ). In an embodiment of an oligomer with n chiral phosphorous-containing linkages (where n is an integer of 1 or greater), the oligomer contains at least 9 contiguous stereopure phosphorous-containing linkages of the same stereo orientation (i.e. either Sₚ or Rₚ). In an embodiment of an oligomer with n chiral phosphorous-containing linkages (where n is an integer of 1 or greater), the oligomer contains at least 10 contiguous stereopure phosphorous-containing linkages of the same stereo orientation (i.e. either Sₚ or Rₚ). In an embodiment of an oligomer with n chiral phosphorous-containing linkages (where n is an integer of 1 or greater), the oligomer contains at least 11 contiguous stereopure phosphorous-containing linkages of the same stereo orientation (i.e. either Sₚ or Rₚ). In an embodiment of an oligomer with n chiral phosphorous-containing linkages (where n is an integer of 1 or greater), the oligomer contains at least 12 contiguous stereopure phosphorous-containing linkages of the same stereo orientation (i.e. either Sₚ or Rₚ). In an embodiment of an oligomer with n chiral phosphorous-containing linkages (where n is an integer of 1 or greater), the oligomer contains at least 13 contiguous stereopure phosphorous-containing linkages of the same stereo orientation (i.e. either Sₚ or Rₚ). In an embodiment of an oligomer with n chiral phosphorous-containing linkages (where n is an integer of 1 or greater), the oligomer contains at least 14 contiguous stereopure phosphorous-containing linkages of the same stereo orientation (i.e. either Sₚ or Rₚ). In an embodiment of an oligomer with n chiral phosphorous-containing linkages (where n is an integer of 1 or greater), the oligomer contains at least 15 contiguous stereopure phosphorous-containing linkages of the same stereo orientation (i.e. either Sₚ or Rₚ). In an embodiment of an oligomer with n chiral phosphorous-containing linkages (where n is an integer of 1 or greater), the oligomer contains at least 16 contiguous stereopure phosphorous-containing linkages of the same stereo orientation (i.e. either Sₚ or Rₚ). In an embodiment of an oligomer with n chiral phosphorous-containing linkages (where n is an integer of 1 or greater), the oligomer contains at least 17 contiguous stereopure phosphorous-containing linkages of the same stereo orientation (i.e. either Sₚ or Rₚ). In an embodiment of an oligomer with n chiral phosphorous-containing linkages (where n is an integer of 1 or greater), the oligomer contains at least 18 contiguous stereopure phosphorous-containing linkages of the same stereo orientation (i.e. either Sₚ or Rₚ). In an embodiment of an oligomer with n chiral phosphorous-containing linkages (where n is an integer of 1 or greater), the oligomer contains at least 19 contiguous stereopure phosphorous-containing linkages of the same stereo orientation (i.e. either Sₚ or Rₚ). In an embodiment of an oligomer with n chiral phosphorous-containing linkages (where n is an integer of 1 or greater), the oligomer contains at least 20 contiguous stereopure phosphorous-containing linkages of the same stereo orientation (i.e. either Sₚ or Rₚ).

In certain embodiments, the antisense oligonucleotides of SEQ ID Nos: 1-10 and 20 in Table 3 may be stereospecific oligomers. In certain embodiments, the antisense oligonucleotide of SEQ ID NO: 1 is a stereospecific oligomer.

9. Morpholino Oligomers

Exemplary embodiments of the disclosure relate to phosphorodiamidate morpholino oligomers of the following general structure:

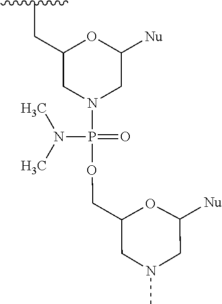

and as described in FIG. 2 of Summerton, J., et al., Antisense & Nucleic Acid Drug Development, 7: 187-195 (1997). Morpholinos as described herein are intended to cover all stereoisomers and tautomers of the foregoing general structure. The synthesis, structures, and binding characteristics of morpholino oligomers are detailed in U.S. Pat. Nos. 5,698,685; 5,217,866; 5,142,047; 5,034,506; 5,166,315; 5,521,063; 5,506,337; 8,076,476; and 8,299,206, all of which are incorporated herein by reference.

In certain embodiments, a morpholino is conjugated at the 5′ or 3′ end of the oligomer with a “tail” moiety to increase its stability and/or solubility. Exemplary tails include:

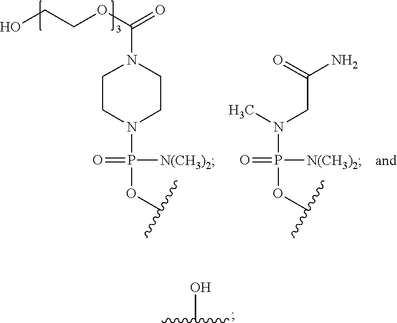

and the distal —OH or —NH₂ of the “tail” moiety is optionally linked to a cell-penetrating peptide.

In certain embodiments, the antisense oligonucleotides of SEQ ID Nos: 1-10 and 20 in Table 3 may be morpholino oligomers. In certain embodiments, the antisense oligonucleotide of SEQ ID NO: 1 is a morpholino oligomer.

10. Nucleobase Modifications and Substitutions

In certain embodiments, antisense oligomers of the disclosure are composed of RNA nucleobases and DNA nucleobases (often referred to in the art simply as “base”). RNA bases are commonly known as adenine (A), uracil (U), cytosine (C) and guanine (G). DNA bases are commonly known as adenine (A), thymine (T), cytosine (C) and guanine (G). In various embodiments, antisense oligomers of the disclosure are composed of cytosine (C), guanine (G), thymine (T), adenine (A), 5-methylcytosine (5mC), uracil (U), and hypoxanthine (I).

In certain embodiments, one or more RNA bases or DNA bases in an oligomer may be modified or substituted with a base other than a RNA base or DNA base. Oligomers containing a modified or substituted base include oligomers in which one or more purine or pyrimidine bases most commonly found in nucleic acids are replaced with less common or non-natural bases.

Purine bases comprise a pyrimidine ring fused to an imidazole ring, as described by the following general formula.

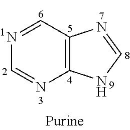

Adenine and guanine are the two purine nucleobases most commonly found in nucleic acids. Other naturally-occurring purines include, but not limited to, N⁶-methyladenine, N²-methylguanine, hypoxanthine, and 7-methylguanine.

Pyrimidine bases comprise a six-membered pyrimidine ring as described by the following general formula.

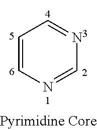

Cytosine, uracil, and thymine are the pyrimidine bases most commonly found in nucleic acids. Other naturally-occurring pyrimidines include, but not limited to, 5-methylcytosine, 5-hydroxymethylcytosine, pseudouracil, and 4-thiouracil. In one embodiment, the oligomers described herein contain thymine bases in place of uracil.

Other suitable bases include, but are not limited to: 2,6-diaminopurine, orotic acid, agmatidine, lysidine, 2-thiopyrimidines (e.g. 2-thiouracil, 2-thiothymine), G-clamp and its derivatives, 5-substituted pyrimidines (e.g. 5-halouracil, 5-propynyluracil, 5-propynylcytosine, 5-aminomethyluracil, 5-hydroxymethyluracil, 5-aminomethylcytosine, 5-hydroxymethylcytosine, Super T), 7-deazaguanine, 7-deazaadenine, 7-aza-2,6-diaminopurine, 8-aza-7-deazaguanine, 8-aza-7-deazaadenine, 8-aza-7-deaza-2,6-diaminopurine, Super G, Super A, and N4-ethylcytosine, or derivatives thereof; N²-cyclopentylguanine (cPent-G), N²-cyclopentyl-2-aminopurine (cPent-AP), and N²-propyl-2-aminopurine (Pr-AP), pseudouracil, or derivatives thereof; and degenerate or universal bases, like 2,6-difluorotoluene or absent bases like abasic sites (e.g. 1-deoxyribose, 1,2-dideoxyribose, l-deoxy-2-O-methylribose; or pyrrolidine derivatives in which the ring oxygen has been replaced with nitrogen (azaribose)). Examples of derivatives of Super A, Super G, and Super T can be found in U.S. Pat. No. 6,683,173 (Epoch Biosciences), which is incorporated here entirely by reference. cPent-G, cPent-AP, and Pr-AP were shown to reduce immunostimulatory effects when incorporated in siRNA (Peacock H. et al. J. Am. Chem. Soc. 2011, 133, 9200). Pseudouracil is a naturally occurring isomerized version of uracil, with a C-glycoside rather than the regular N-glycoside as in uridine. Pseudouridine-containing synthetic mRNA may have an improved safety profile compared to uridine-containing mPvNA (WO 2009127230, incorporated here in its entirety by reference).

Certain nucleobases are particularly useful for increasing the binding affinity of the antisense oligomers of the disclosure. These include 5-substituted pyrimidines, 6-azapyrimidines, and N-2, N-6, and O-6 substituted purines, including 2-aminopropyladenine, 5-propynyluracil, and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications. Additional exemplary modified nucleobases include those wherein at least one hydrogen atom of the nucleobase is replaced with fluorine.

In certain embodiments, the antisense oligonucleotides of SEQ ID Nos: 1-10 and 20 in Table 3 may contain one or more nucleobase modification or substitution. In certain embodiments, the antisense oligonucleotide of SEQ ID NO: 1 may contain one or more nucleobase modification or substitution.

D. Use to Restore the Dystrophin Reading Frame by Exon Skipping

Normal dystrophin mRNA containing all 79 exons will produce normal dystrophin protein. The graphic in FIG. 2 depicts a small section of the dystrophin pre-mRNA and mature mRNA, from exon 47 to exon 53. The shape of each exon depicts how codons are split between exons; of note, one codon consists of three nucleotides. Rectangular shaped exons start and end with complete codons. Arrow shaped exons start with a complete codon but end with a split codon, containing only nucleotide #1 of the codon. Nucleotides #2 and #3 of this codon are contained in the subsequent exon which will start with a chevron shape.

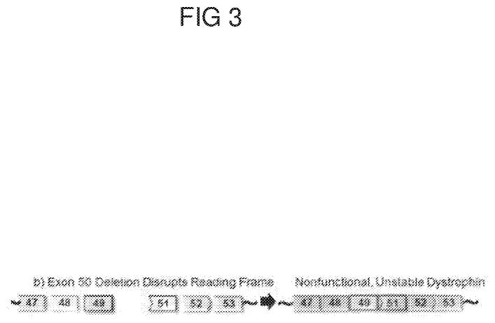

Dystrophin mRNA missing whole exons from the dystrophin gene typically result in DMD. The graphic in FIG. 3 illustrates a type of genetic mutation (deletion of exon 50) that is known to result in DMD. Since exon 49 ends in a complete codon and exon 51 begins with the second nucleotide of a codon, the reading frame after exon 49 is shifted, resulting in out-of-frame mRNA reading frame and incorporation of incorrect amino acids downstream from the mutation. The subsequent absence of a functional C-terminal dystroglycan binding domain results in production of an unstable dystrophin protein.

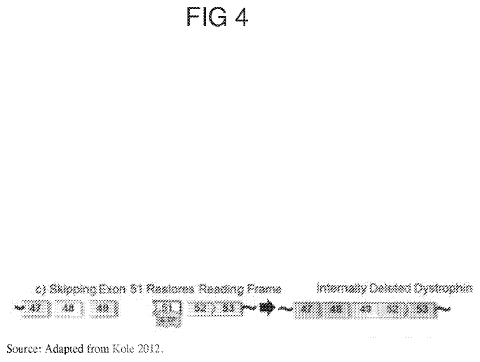

Another exon skipping PMO, eteplirsen, skips exon 51 to restore the mRNA reading frame. Since exon 49 ends in a complete codon and exon 52 begins with the first nucleotide of a codon, deletion of exon 51 restores the reading frame, resulting in production of an internally-shortened dystrophin protein with an intact dystroglycan binding site, similar to an “in-frame” BMD mutation (FIG. 4).

The feasibility of ameliorating the DMD phenotype using exon skipping to restore the dystrophin mRNA open reading frame is supported by nonclinical research. Numerous studies in dystrophic animal models of DMD have shown that restoration of dystrophin by exon skipping leads to reliable improvements in muscle strength and function (Sharp 2011; Yokota 2009; Wu 2008; Wu 2011; Barton-Davis 1999; Goyenvalle 2004; Gregorevic 2006; Yue 2006; Welch 2007; Kawano 2008; Reay 2008; van Putten 2012). A compelling example of this comes from a study in which dystrophin levels following exon skipping (using a PMO) therapy were compared with muscle function in the same tissue. In dystrophic mdx mice, tibialis anterior (TA) muscles treated with a mouse-specific PMO maintained ˜75% of their maximum force capacity after stress-inducing contractions, whereas untreated contralateral TA muscles maintained only ˜25% of their maximum force capacity (p<0.05) (Sharp 2011). In another study, 3 dystrophic CXMD dogs received at (2-5 months of age) exon-skipping therapy using a PMO-specific for their genetic mutation once a week for 5 to 7 weeks or every other week for 22 weeks. Following exon-skipping therapy, all 3 dogs demonstrated extensive, body-wide expression of dystrophin in skeletal muscle, as well as maintained or improved ambulation (15 m running test) relative to baseline. In contrast, untreated age-matched CXMD dogs showed a marked decrease in ambulation over the course of the study (Yokota 2009).

PMOs were shown to have more exon skipping activity at equimolar concentrations than phosphorothioates in both mdx mice and in the humanized DMD (hDMD) mouse model, which expresses the entire human DMD transcript (Heemskirk 2009). In vitro experiments using reverse transcription polymerase chain reaction (RT-PCR) and Western blot (WB) in normal human skeletal muscle cells or muscle cells from DMD patients with different mutations amenable to exon 51 skipping identified eteplirsen as a potent inducer of exon 51 skipping. Eteplirsen-induced exon 51 skipping has been confirmed in vivo in the hDMD mouse model (Arechavala-Gomeza 2007).

Clinical outcomes for analyzing the effect of an antisense oligonucleotide that specifically hybridizes to an exon 53 target region of the Dystrophin pre-mRNA and induces exon 53 skipping include an increase from baseline of percent normal dystrophin , six-minute walk test (6MWT), loss of ambulation (LOA), North Star Ambulatory Assessment (NSAA), pulmonary function tests (PFT), ability to rise (from a supine position) without external support, de novo dystrophin production and other functional measures.

E. Clinical Findings and Outcomes for Golodirsen Administration

Golodirsen (SRP-4053) is being evaluated in an ongoing Phase I/II clinical study (Study 4053-101) in patients who have a confirmed mutation of the DMD gene that is amenable to exon 53 skipping.

Study 4053-101 is a Phase I/II study of SRP-4053 (golodirsen) in DMD patients. This study is a 2-Part, Randomized, Double-Blind, Placebo-Controlled, Dose-Titration, Safety, Tolerability, and Pharmacokinetics Study (Part 1) Followed by an Open-Label Efficacy and Safety Evaluation (Part 2) of SRP-4053 in Patients with Duchenne Muscular Dystrophy Amenable to Exon 53 Skipping. Primary outcome measures include Incidence of Adverse Events [Time Frame: approximately 12 weeks (Part 1)], Change in 6-Minute Walk Test (6MWT) from Baseline [Time Frame: 144 weeks (Part 2)], and Percentage of dystrophin-positive fibers [Time Frame: 48 weeks (Part 2)]. Secondary outcome measures include Drug concentration in plasma [Time Frame: Approximately 12 weeks (Part 1)], Maximum inspiratory pressure (MIP) % predicted, maximum expiratory pressure (MEP) % predicted [Time Frame: 144 weeks (Part 2)].

Further details of this study are found on www.clinicaltrials.org (NCT02310906).

Data from NCT02310906 is described in U.S. Ser. No. 62/553,094, which is incorporated herein by reference. In particular, RT-PCR analysis was performed to confirm exon skipping DMD patients. A summary of the RT-PCR results is shown in Table 2. All 25 patients who received at least 48 weekly doses of SRP-4053 displayed an increase over baseline levels in exon skipping (p<0.001).

TABLE 2
RT-PCR Results Confirm Exon Skipping in DMD Patients
RT-PCR Results

| | Part 2/Pl patients | Part 1 patients | |
|---|---|---|---|
| | 48-51 weeks | 60-76 weeks | All patients |
| No Increase > 0.1 | 0 | 0 | 0 |
| from Baseline | | | |
| Increase > 0.1 | 17 (100.0%) | 8 (100.0%) | 25 (100.0%) |
| from Baseline | | | |
| 95% CI | (80.5%, 100.0%) | (63.1%, 100.0%) | (86.3%, 100.0%) |
| Decrease from | 0 | 0 | 0 |
| Baseline | | | |
| Unchanged from | 0 | 0 | 0 |
| Baseline | | | |
| Increase from | 17 (100.0%) | 8 (100.0%) | 25 (100.0%) |
| Baseline | | | |
| P-value | <0.001 | 0.008 | <0.001 |
| Fold Increase | — | — | 7.3 |

In addition, Western blot analysis was performed to confirm dystrophin production in DMD patients. A summary of the Western blot results is shown in Table 5. Patients demonstrated a statistically significant increase over baseline in dystrophin protein as measured by Western blot.

TABLE 5
Western Blot Results Confirm Dystrophin Production in DMD Patients
Western Blot Results

| | Part 2 patients | Part 1 patients | |
|---|---|---|---|
| | ~48-51 weeks | 60-76 weeks | All |
| | dosing | dosing | patients |
| n | 17 | 8 | 25 |
| Baseline Mean % | 0.09 (0.06) | 0.10 (0.09) | 0.09 (0.07) |
| normal (SD) | | | |
| On treatment | 0.84 (0.64) | 1.40 (1.57) | 1.02 (1.03) |
| Mean % normal (SD) | | | |
| Mean Change from | 0.75 (0.67) | 1.29 (1.51) | 0.92 (1.01) |
| baseline (SD) | | | |
| P-value | <0.001 | 0.008 | P < 0.001 |
| Fold Increase | — | — | |

A positive correlation between exon skipping and de novo dystrophin protein was observed (Spearman-r=0.500, p=0.011).

Analysis of mean fiber intensity demonstrated a statistically significant increase (p<0.001) above baseline in de novo dystrophin and that dystrophin was correctly localized to the sarcolemma membrane.

Exon skipping and sarcolemmal dystrophin localization were observed in all patients.

F. Study 4045-301 (ESSENCE):

Study 4045-301 is a study of SRP-4045 (casimersen) and SRP-4053 (golodirsen) in DMD patients. This study is a double-blind, placebo-controlled, multi-center, 48-week study to evaluate the efficacy and safety of SRP-4045 and SRP-4053. Eligible patients with out-of-frame deletions that may be corrected by skipping exon 45 or 53 will be randomized to receive once weekly intravenous (IV) infusions of 30 mg/kg SRP-4045 or 30 mg/kg SRP-4053 respectively (combined-active group, 66 patients) or placebo (33 patients) for 48 weeks. Clinical efficacy will be assessed at regularly scheduled study visits, including functional tests such as the six minute walk test. All patients will undergo a muscle biopsy at Baseline and a second muscle biopsy over the course of the study. Safety will be assessed through the collection of adverse events (AEs), laboratory tests, electrocardiograms (ECGs), echocardiograms (ECHOs), vital signs, and physical examinations throughout the study. Blood samples will be taken periodically throughout the study to assess the pharmacokinetics of both drugs. Primary outcome measures include Change in 6 Minute Walk Test (6MWT) from Baseline [Time Frame: Baseline to Week 48] and secondary outcome measures include percent normal dystrophin[Time Frame: Baseline to Week 24 and 48 ] and Change in maximum inspiratory pressure (MIP) % predicted, maximum expiratory pressure (MEP) % predicted from Baseline [ Time Frame: Baseline to Week 48]. Further details of this study are found on www.clinicaltrials.org (NCT02500381).

6 Minute Walk Test

Given the pivotal role of ambulation in daily human function and the impact of its inevitable loss in DMD, the 6MWT at year three can be considered an “intermediate” clinical efficacy outcome for Accelerated Approval.

The 6MWT assessments are conducted in a standardized manner according to international guidelines.

Loss of Ambulation

Ambulatory compromise and irreversible loss of ambulation (LOA) are hallmarks of the progressive muscle degeneration characteristic of DMD. It is a reliable overall indicator of the severity of disease progression and strongly correlates with functional measures such as the 6MWT; it is also less influenced by motivational factors. Furthermore, LOA predicts other major disease milestones such as the need for ventilatory support and survival (Bello 2016). Once confined to a wheelchair, other symptoms tend to follow in rapid succession.

Northstar Ambulatory Assessment (NSAA)

The NSAA is a clinician-reported outcome instrument specifically designed to measure function in ambulatory patients with DMD. The 17 items are each scored on a 0-2 ordinal scale and include assessments of abilities such as rising from the floor, climbing and descending a step, 10 meter walk/run and lifting the head.

Ability to Rise without External Support

The ability to rise from supine is a critical activity for DMD patients, is one of the early abilities to be lost and may be predictive of loss of ambulation. It has been suggested that the loss of ability to rise may predict loss of ambulation within 1-2 years.

Pulmonary Function Tests

Respiratory function in DMD is progressively impaired over time as the dystrophic process affects respiratory muscles, including the diaphragm, leading to significant morbidity and mortality. Treated boys tend to have slower deterioration of respiratory muscle function as measured by FVC %predicted when compared to baseline data or natural history data. Additionally, MEP %predicted and MIP %predicted may also decline more slowly with treatment than expected, although the scientific literature on these parameters is more limited.

Antisense Oligonucleotides and Alternative Chemistries

In other embodiments, additional antisense oligonucleotides for use in the present disclosure may be selected from the sequences shown as SEQ ID Nos. 1-10 and 20 in Table 3. In some embodiments, antisense oligonucleotides for use in the present disclosure are found in WO 2004/083432, WO 2012/029986, U.S. Pat. No. 8,084,601, WO 2012/109296, each of which is incorporated herein by reference.

Antisense oligonucleotides may be generated using different chemistries. For example, besides being a PMO, the antisense oligonucleotide may be a 2′-O-methyl-phosphorothioate, i.e., an AON in which the each and every nucleotide in the oligonucleotide is modified at the 2′-position such that the resulting structure has a methoxy group at the 2′-position and all nucleotides in the oligonucleotide are joined by phosphorothioate linkages (in place of phosphodiester linkages found in naturally-occurring RNA and DNA). FIG. 1, where R is methoxy (i.e., -OCH₃) represents the chemical structure of a 2′-O-methyl-phosphorothioate. Drisapersen is an example of a 2′-O-methyl-phosphorothioate antisense oligonucleotide.

Phosphorothioates are known to cause a number of other target organ toxicities in animals, including complement activation and pro-inflammatory effects, coagulopathies, thrombocytopenia, vascular injury, and hepatic Kuppfer cell basophilia (Levin 1998; Monteith 1999; Levin 2001; Henry 2008; Frazier 2014; Engelhardt 2015; Frazier 2015). Thorough evaluations of the developing immune system in juvenile rats, which included T cell-dependent antibody responses and immunophenotyping of peripheral blood T- and B-cell subpopulations (total/helper/cytotoxic T-cells, B-cells, and NK cells), demonstrated that eteplirsen, a PMO, had no adverse effect on the immune response.

In addition to being a morpholino or a 2′-O-methyl-phosphorothioate, the antisense oligonucleotides of the disclosure may also be a peptide nucleic acid (PNA), a locked nucleic acid (LNA), or a bridged nucleic acid (BNA) such as 2′-O,4′-C-ethylene-bridged nucleic acid (ENA).

In some embodiments, the present disclosure provides antisense oligonucleotides capable of binding to a selected target in the dystrophin pre-mRNA to induce efficient and consistent skipping of exon 53. Duchenne muscular dystrophy arises from mutations that preclude the synthesis of a functional dystrophin gene product. These Duchenne muscular dystrophy gene defects are typically nonsense mutations or genomic rearrangements such as deletions, duplications or micro-deletions or insertions that disrupt the reading frame. As the human dystrophin gene is a large and complex gene with the 79 exons being spliced together to generate a mature mRNA with an open reading frame of approximately 11,000 bases, there are many positions where these mutations can occur. Consequently, a comprehensive antisense oligonucleotide based therapy to address many of the different disease-causing mutations in the dystrophin gene will require that many exons can be targeted for removal during the splicing process. Furthermore, the antisense oligonucleotide based therapy may be administered with a non-steroidal anti-inflammatory compound.

Exemplary embodiments of the disclosure relate to morpholino oligonucleotides having phosphorodiamidate backbone linkages. Morpholino oligonucleotides with uncharged backbone linkages, including antisense oligonucleotides, are detailed, for example, in (Summerton and Weller 1997) and in co-owned U.S. Pat. Nos. 5,698,685, 5,217,866, 5,142,047, 5,034,506, 5,166,315, 5,185, 444, 5,521,063, 5,506,337, 8,076,476, and 8,299,206 all of which are expressly incorporated by reference herein.

Important properties of the morpholino-based subunits include: 1) the ability to be linked in a oligomeric form by stable, uncharged backbone linkages; 2) the ability to support a nucleotide base (e.g. adenine, cytosine, guanine, thymidine, uracil and inosine (hypoxanthine)) such that the polymer formed can hybridize with a complementary-base target nucleic acid, including target RNA, Tm values above about 45° C. in relatively short oligonucleotides (e.g., 10-15 bases); 3) the ability of the oligonucleotide to be actively or passively transported into mammalian cells; and 4) the ability of the antisense oligonucleotide:RNA heteroduplex to resist RNAse and RNase H degradation, respectively.

In certain embodiments, the antisense compounds can be prepared by stepwise solid-phase synthesis, employing methods detailed in the references cited above, and below. In some cases, it may be desirable to add one or more additional chemical moieties to the antisense compound, e.g., to enhance pharmacokinetics or to facilitate capture or detection of the compound. Such a moiety, such as a tail moiety described herein, may be covalently attached, according to standard synthetic methods. For example, addition of a polyethylene glycol moiety or other hydrophilic polymer, e.g., one having 1-100 monomeric subunits, may be useful in enhancing solubility.

A reporter moiety, such as fluorescein or a radiolabeled group, may be attached for purposes of detection. Alternatively, the reporter label attached to the oligomer may be a ligand, such as an antigen or biotin, capable of binding a labeled antibody or streptavidin. In selecting a moiety for attachment or modification of an antisense compound, it is generally of course desirable to select chemical compounds of groups that are biocompatible and likely to be tolerated by a subject without undesirable side effects.

Oligomers for use in antisense applications generally range in length from about 10 to about 50 subunits. In some embodiments, antisense oligomers of the disclosure range in length from about 10 to 30 subunits including, for example, 15,16, 17, 18, 19, 20, 21, 22, 23, 24, 25, 26, 27, 28, 29, 30, 31, 32, 33, 34 or 35 subunits. In various embodiments, the oligomers of the disclosure have 25 to 28 subunits.

Each morpholino ring structure supports a base pairing moiety, to form a sequence of base pairing moieties which is typically designed to hybridize to a selected antisense target in a cell or in a subject being treated. The base pairing moiety may be a purine or pyrimidine found in native DNA or RNA (e.g., A, G, C, T or U) or an analog, such as hypoxanthine (the base component of the nucleoside inosine) or 5-methyl cytosine.

The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target.

It is understood in the art that the sequence of an antisense molecule need not be 100% complementary to that of its target sequence to be specifically hybridizable. An antisense molecule is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or treatment, and in the case of in vitro assays, under conditions in which the assays are performed.

While the above method may be used to select antisense molecules capable of deleting any exon from within a protein that is capable of being shortened without affecting its biological function, the exon deletion should not lead to a reading frame shift in the shortened transcribed mRNA. Thus, if in a linear sequence of three exons the end of the first exon encodes two of three nucleotides in a codon and the next exon is deleted then the third exon in the linear sequence must start with a single nucleotide that is capable of completing the nucleotide triplet for a codon. If the third exon does not commence with a single nucleotide there will be a reading frame shift that would lead to the generation of truncated or a non-functional protein.

It will be appreciated that the codon arrangements at the end of exons in structural proteins may not always break at the end of a codon, consequently there may be a need to delete more than one exon from the pre-mRNA to ensure in-frame reading of the mRNA. In such circumstances, a plurality of antisense oligonucleotides may need to be selected by the method of the disclosure wherein each is directed to a different region responsible for inducing splicing in the exons that are to be deleted.

To avoid degradation of pre-mRNA during duplex formation with the antisense molecules, the antisense molecules used in the method may be adapted to minimize or prevent cleavage by endogenous RNase H. This property is highly preferred as the treatment of the RNA with the unmethylated oligonucleotides either intracellularly or in crude extracts that contain RNase H leads to degradation of the pre-mRNA: antisense oligonucleotide duplexes. Any form of modified antisense molecules that is capable of by-passing or not inducing such degradation may be used in the present method. An example of antisense molecules which when duplexed with RNA are not cleaved by cellular RNase H is 2′-O-methyl derivatives. 2′-O-methyl-oligoribonucleotides are very stable in a cellular environment and in animal tissues, and their duplexes with RNA have higher Tm values than their ribo- or deoxyribo-counterparts.

While antisense oligonucleotides are a preferred form of the antisense molecules, the present disclosure comprehends other oligomeric antisense molecules, including but not limited to oligonucleotide mimetics.

In various embodiments, antisense compounds useful in this disclosure include oligonucleotides containing modified backbones or non-natural inter-nucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their inter-nucleoside backbone can also be considered to be oligonucleosides.

It is not necessary for all positions in a given compound to be uniformly modified, and in fact more than one of the aforementioned modifications may be incorporated in a single compound or even at a single nucleoside within an oligonucleotide.

G. Non-Steroidal Anti-Inflammatory Compounds

According to one aspect of the disclosure, there is provided non-steroidal anti-inflammatory compounds capable of treating or reducing inflammation, and/or enhancing muscle regeneration in a subject with Duchenne muscular dystrophy (DMD). In some embodiments, the non-steroidal anti-inflammatory compounds are NF-κB inhibitors.

Duchenne muscular dystrophy is characterized by progressive muscle degeneration and is caused by dystrophin gene mutations that preclude the synthesis of a functional dystrophin gene product. The absence of functional dystrophin results in muscle fibers that are prone to mechanical stress, inflammation of muscle cells, muscle damage, and reduced ability to regenerate muscle tissue. Consequently, non-steroidal anti-inflammatory based therapy administered with antisense oligonucleotide based therapy may address the symptoms of DMD that are caused by inflammation as well as targeting and removing the disease causing mutations in the dystrophin gene.

NF κB Inhibitors

NF-κB is a molecule that is activated in Duchenne's Muscular Dystrophy (DMD) as well as other skeletal muscle disorders and rare diseases. The absence of dystrophin in DMD triggers an increase in NF-κB levels as a result of injury to muscle cell membranes (Donovan, J. (2014)). Elevated NF-κB levels lead to inflammation, tissue damage, and fibrosis, all of which contribute to muscle degeneration and decreased muscle mass in DMD patients. Furthermore, the activation of this signaling molecule results in muscle damage and prevents muscle regeneration.

NF-κB is a family of transcription factors that exists in a cytoplasmic complex with IκB in unstimulated cells (see, e.g., Gilmore, T. D. (2006) Oncogene 25, 6680-6684). Stimulation results in the phosphorylation of Ix13, which leads to its degradation and allows free NF-κB to translocate to the nucleus and activate target genes (Gilmore, T. D. (2006)). Targets that are regulated by NF-κB include pro-inflammatory cytokines, such as TNF-α, IL-6, and IL-1β, and enzymes such as cyclooxygenase-2. Activation of NF-κB can be blocked by mechanisms that prevent IκB degradation and cause NF-κB to be retained in the cytoplasm. For example, degradation of IκB can be blocked pharmacologically by salicylate, which inhibits IKKβ, a kinase that phosporylates IκB, or genetically by the use of a phosphorylation-resistant variant of IκB (Kopp, E. and Ghosh, S. (1994) Science 265, 956-959; Van Antwerp, D. J., et al., (1996) Science 274, 787-789).

The activation of NF-kB results in the degradation of muscle proteins and the induction of pro-inflammatory mediators such as cytokines (e.g., tumor necrosis factor-α (TNF-α), interleukin-6 (IL-6), interleukin-β (IL-β), chemokines, cell adhesion molecules, and tissue degrading enzymes (e.g., matrix metallopeptidase 9 (MMP-9). The activation of NF-κB also suppresses muscle stem cell differentiation, which is needed for muscle regeneration. Specifically, the activation of NF-κB prevents satellite stem cells from differentiating into myoblasts, which are progenitor cells that differentiate to give rise to muscle cells.

In DMD patients, the activation of NF-κB is observed in muscle tissue prior to the onset of other clinical manifestations. In addition, the immune cells and degenerating muscle fibers of DMD patients continually show elevated levels of activated NF-κB. Evidence also suggests that mechanical stress activates NF-κB in muscle and drives NF-κB mediated inflammation. More rapid deterioration of muscle is observed in muscles with increased mechanical stress and inflammation; for example, quadriceps and hamstrings.

Inhibitors of NF-κB may be used to reduce muscle inflammation and enhance muscle regeneration in patients with DMD. Thus, NF-κB inhibitors may provide a benefit to DMD patients by allowing them to retain muscle function for a longer period of time. Agents that reduce NF-κB activity or otherwise block muscle degeneration and/or promote muscle regeneration can be useful in the treatment of DMD, either by themselves or as a combination therapy with other agents that restore dystrophin expression.

Examples of NF-κB inhibitors include NF-kappa B pathway inhibitors, p105-based NF-kappa B super repressor, IMS-088, cimetidine+cyclophosphamide+diclofenac+sulfasalazine, nanocurcumin, denosumab, SCB-633, recombinant anti-RANK-L mAb, recombinant human lymphotoxin derivatives, POP 2, curcumin and resveratrol analogs, NFW9C-25, IB-RA, SKLB-023, KPT-350, EC-70124, REM-1086, AMG-0102, SGD-2083, tarenflurbil, NF-kB inhibitors, cobitolimod, curcumin analogs, CBL-0137, FE-999301, anticancer therapeutics, SPA-0355, KIN-219, NFkappaB decoy oligo program, bardoxolone methyl, TAK1-NF-kBNF-kB inhibitors, S-414114, mesalamine+N-acetylcysteine, CU-042, dual p53-mdm2/NF-kappaB inhibitors, TNF alpha.NF-kB inhibitors, liposomal curcumin, CBL-0137, IB-RA, CPC-551, IMD-0560, AMG-0103, AκBA, KD-018, azelaic acid, mepacrine, NBD peptides, triflusal, KN-013, HMPL-004, IMD-1041, PPL-003, RGN-352, RGN-137. Additional examples of NF-kB inhibitors include edasalonexent (CAT-1004) and CAT-1041. In one embodiment, the NF-kB inhibitor is edasalonexent.

Edasalonexent and CAT-1041 belong to a novel class of orally bioavailable NF-κB inhibitors for the treatment of dystrophic muscle. These compounds are composed of a polyunsaturated fatty acid (PUFA) and salicylic acid, which individually inhibit the activation of cNF-κB, conjugated together by a linker that is only susceptible to hydrolysis by intracellular fatty acid hydrolase. These compounds have been shown to inhibit cNF-κB activation in vitro, and that long-term treatment improves the phenotype of both the mdx mouse and golden retriever muscular dystrophy (GRMD) dog models of DMD (Hammers et al., JCI Insight, 2016; 1(21):e90341. In some embodiments, this class of NF-κB inhibitors can serve as an effective treatment to slow disease progression in DMD patients.

TNFα-mediated regulation of microRNAs that negatively control dystrophin expression has been observed (Fiorillo et al. Cell reports 2015). In particular, TNFα increases dystrophin regulating microRNAs (Fiorillo et al. Cell reports 2015). Therefore, in some embodiments, inhibition of NF-kB should downregulate TNFα and allow for enhanced dystrophin expression in Becker muscular dystrophy patients. DMD patients have essentially no dystrophin expression and, in some embodiments, a combinatorial treatment regimen with a dystrophin restoring agent (e.g., a PMO) and an NF-kB inhibitor may be used to enhance dystrophin expression.

a. Fatty Acid Acetylated Salicylates

Fatty acid acetylated salicylates are compounds that can inhibit NF-κB activity and reduce inflammation (see U.S. Pat. No. 8,173,831, incorporated herein by reference). This class of compounds includes bifunctional small molecules comprising salicylate and omega-3 polyunsaturated fatty acids (PUFAs) joined by a chemical linker. Structurally, a subclass of these compounds can be described by the formula:

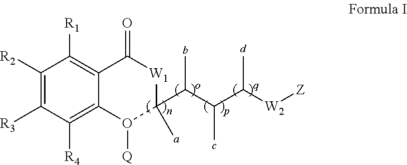

wherein
R₁, R₂, R₃, and R₄ are each independently selected from the group consisting of H, Cl, F, CN, NH₂, —NH(C₁-C₃ alkyl), —N(C₁-C₃ alkyl)₂, —NH(C(O)C₁-C₃ alkyl), —N(C(O)C₁-C₃alkyl)₂, —C(O)H, —C(O)C₁-C₃ alkyl, —C(O)0C₁-C₃ alkyl, —C(O)NH₂, —C(O)NH(C₁-C₃ alkyl), —C(O)N(C₁-C₃alkyl)₂, —C₁-C₃ alkyl, —O—C₁-C₃ alkyl, —S(O)C₁-C₃ alkyl, and —S(O)₂C₁-C₃ alkyl;
W₁ and W₂ are each independently null, O, or NH, or when W₁ and W₂ are both NH, then both W₁ and W₂ can be taken together to form a piperidine moiety;
- - - represents an optional bond that when present requires that Q is null;
a and c are each independently H, CH₃, —OCH₃, —OCH₂CH₃, or C(O)OH;
b is H, CH₃, C(O)OH, or O—Z;
d is H or C(O)OH;
each n, o, p, and q is independently 0 or 1;
each Z is H or

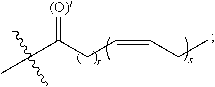

with the proviso that there is at least one

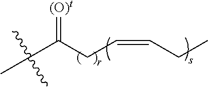

in the compound;
each r is independently 2 or 3;
each s is independently 5 or 6;
each t is independently 0 or 1;
Q is null, C(O)CH₃, Z,

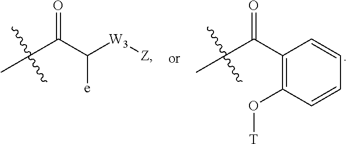

e is H or any one of the side chains of the naturally occurring amino acids;
W₃ is null, —O—, or —N(R)—;
R is H or C₁-C₃ alkyl; and
T is H, C(O)CH₃, or Z. In a subclass of these compounds, W₂ is NH. In a further subclass, r is 2, s is 6, and Z is

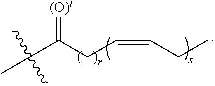

Synthesis of fatty acid acetylated salicylates is described generally in WO 2010/006085 A1, which is hereby incorporated by reference in its entirety.

A key advantage of fatty acid acetylated salicylates in fighting inflammation is the ability of their component parts to function synergistically (see U.S. Pat. No. 8,173,831). Chemical linkers are chosen that are resistant to extracellular degradation but can be cleaved by intracellular enzymes (see U.S. Pat. No. 8,173,831). The chemical linkers attach to portions of salicylate and the omega-3 PUFA that prevent these molecules from exerting their pharmacological effects. Consequently, intact fatty acid acetylated salicylates are inactive, which reduces off-target effects when the compounds are in circulation. Upon entry into a target cell, however, degradation of the chemical linker results in the release of salicylate and the omega-3 PUFA. Salicylate prevents degradation of IκB, which retains NF-κB in the cytoplasm and blocks transcription of pro-inflammatory factors, such as cytokines (Kopp, E. and Ghosh, S. (1994)). Omega-3 PUFAs increase anti-inflammatory cytokines, such as IL-10, and adipokines, such as adiponectin. Increased levels of circulating omega-3 PUFAs correlate with lower levels of TNF-α and IL-6 (Ferrucci, L. et al., (2006) J. Clin. Endocrin. Metab. 91, 439-446). Whereas salicylate and an omega-3 PUFA might enter different cells or tissues when administered separately, fatty acid acetylated salicylates allow the two active molecules to be targeted to the same cells. In addition, because salicylate inhibits pro-inflammatory pathways while the omega-3 PUFA activates anti-inflammatory pathways, fatty acid salicylates prevent inflammation more effectively than do compounds that target just one set of regulatory pathways.

i. Edasalonexent

An example of a fatty acid acetylated salicylate with high therapeutic potential is edasalonexent, also referred to as CAT-1004 (Milne, J. et al., Neuromuscular Disorders, Volume 24, Issue 9, 825 (2014)). N-(2-[(4Z,7Z,10Z,13Z,16Z,19Z)-docosa-4,7,10,13,16,19-hexaenamido]ethyl)-2-hydroxybenzamide], is an orally administered novel small molecule in which salicylic acid and docosahexaenoic acid (DHA) are covalently conjugated through an ethylenediamine linker and that is designed to synergistically leverage the ability of both of these compounds to inhibit NF-κB. CAT-1004, a code name, is also known by its international non-proprietary name “edasalonexent” and is reported to be assigned CAS Registry No. 1204317-86-1 and having the following structure:

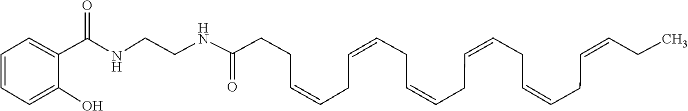 [SMILES: CC/C=C\C/C=C\C/C=C\C/C=C\C/C=C\C/C=C\CCC(=O)NCCNC(=O)c1ccccc1O]

### WHO Drug Information, Vol. 29, No. 4, 2015.

In some embodiments, CAT-1004 can be formulated for oral delivery, for example, in capsules, as described in U.S. Pat. No. 8,173,831, incorporated herein by reference. The PUFA in CAT-1004 is docosahexaenoic acid (DHA) (Milne, J. et al., (2014)). Omega-3 DHA triggers anti-inflammatory pathways via multiple mechanisms (see, e.g., Chapkin, et al., (2009) Prostaglandins Leukot. Essent. Fatty Acids 81, 187-191). CAT-1004 has been shown to enhance muscle regeneration, reduce muscle degeneration and inflammation, and preserve muscle function in mdx mice Milne, J. et al., (2014)). In long-term studies on mdx mice, CAT-1004 treatment results in improved diaphragm function and increased cumulative run distance (Milne, J. et al., (2014)). In a dog model of DMD, CAT-1004 decreases NF-κB activity as evidenced by reduced binding of the p65 subunit to DNA and reduced secretion of the inflammatory mediator TNF-α. In humans, administration of CAT-1004 results in a decrease of biomarkers of inflammation in whole blood. In healthy adult humans, CAT-1004 treatment also lowers levels of the p65 subunit of NF-κB compared to treatment with a placebo or with salicylate and omega-3 DHA as separate molecules.

In some embodiments, treatment is measured by assaying the serum of DMD patients for biomarkers of inflammation. In some embodiments, the treatment results in a reduction in the levels of one or more, or a combination of biomarkers of inflammation. For example, in some embodiments, the biomarkers of inflammation are one or more or a combination of the following: cytokines (such as IL-1, IL-6, TNF-α), C-reactive protein (CRP), leptin, adiponectin, and creatine kinase (CK). In some embodiments, treatment lowers levels of the p65 subunit of NF-κB compared to treatment with a placebo or with salicylate and omega-3 DHA as separate molecules. In some embodiments, biomarkers of inflammation are assayed by methods known in the art; for example, see Rocio Cruz-Guzman et al., BioMed Research International, 2015, incorporated herein by reference. It is contemplated that treatment results in a reduction in the level of one or more of the foregoing biomarkers by at least 5%, 10%, 15%, 20%, 25%, 30%, 35%, 40%, 45%, 50%, 55%, 60%, 65%, 70%, 75%, 80%, 85%, 90%, 95%, 99%, or 100% relative to the level of the biomarker prior to treatment.

ii. CAT-1041

Another fatty acid acetylated salicylate of potential therapeutic value is CAT-1041 . CAT-1041 is a homolog and structurally similar to CAT-1004 but has eicosapentaenoic acid (EPA) as its PUFA moiety. In long-term studies on mdx mice, CAT-1041 treatment preserves muscle function, increases skeletal muscle weight, and reduces muscle fibrosis. CAT-1041 may also reduce cardiomyopathy in mdx mice.

b. Synthesis of CAT-1004

The synthesis of CAT-1004 is described in WO 2010/006085 A1, the contents of which are hereby incorporated herein by reference for all purposes. Ethylenediamine is dissolved in water containing bromoaresal

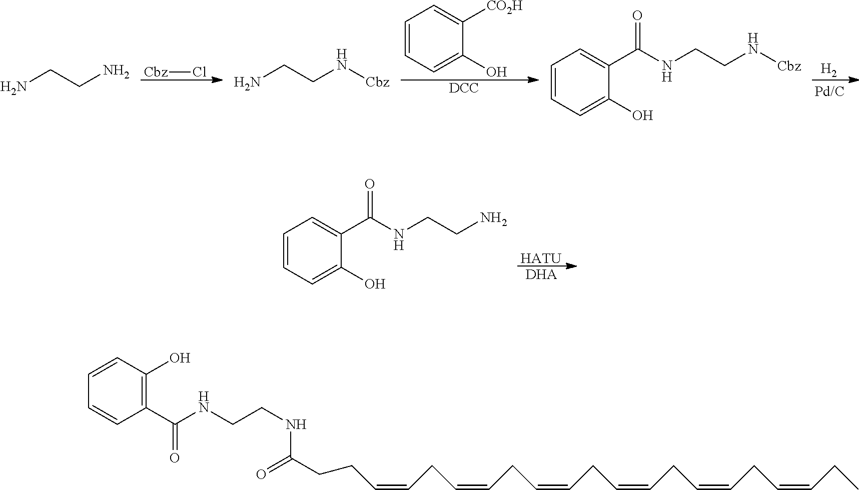

green as an indicator. Methane sulfonic acid in water is added until a blue to pale yellow color transition is just achieved. The solution is diluted with ethanol and vigorously stirred. To the mixture is added the solution of Cbz-CI in dimethoxy ethane and 50% w/v aqueous AcOK at 20° C. simultaneously to maintain the pale yellow-green color of the indicator. After the additions are complete the mixture is stirred and concentrated at low temperature under vacuum to remove the volatiles. The residue is shaken with water and filtered. The filtrate is then washed with toluene, basified with excess 40% aqueous NaOH and extracted with toluene. The organic layer is washed with brine, dried over Na₂SO₄ and evaporated to give benzyl 2-aminoethylcarbamate as an oil.

To a mixture of benzyl 2-aminoethylcarbamate, imidazole, salicylic acid in ethyl acetate is added a solution of DCC in ethyl acetate. The mixture is stirred and filtered. The solution is concentrated under reduced pressure and the crude product is purified by silica chromatography to afford benzyl 2-(2-hydroxybenzamido)ethylcarbamate as a white solid.

A mixture of benzyl 2-(2-hydroxybenzamido)ethylcarbamate and Pd/C in MeOH is stirred under a H₂ atmosphere. The mixture is filtered and concentrated under reduced pressure. The crude product is purified by silica chromatography to afford N-2-(aminoethyl)2-hydroxybenzamide as a white powder.

To a mixture of N-2-(aminoethyl)2-hydroxybenzamide, DHA and Et₃N in CH₃CN is added HATU. The mixture is stirred and concentrated under reduced pressure. The residue is treated with brine and extracted with EtOAc. The combined organic layers are washed with 1M HCl, brine, 5% NaHCO₃ and brine. The organic solution is dried over MgSO₄ and concentrated under reduced pressure. The crude product is purified by silica chromatography to afford N-(2-docosa-4, 7, 10, 13, 16, 19-hexaenamidoethyl)-2-hydroxybenzamide as light yellow oil.

H. mdx Mouse Model of DMD

The mdx mouse is a useful and generally accepted animal model for studying Duchenne's muscular dystrophy (DMD) (Mann et al., Proc. Natl. Acad. Sci., 2001, Jan 2:98(1):42-7, the contents of which are hereby incorporated herein by reference for all purposes). mdx mice are deficient in expression of full-length dystrophin due to a genetic mutation within the dystrophin gene. In particular, mdx dystrophic mice carry a mutation in exon 23 of the dystrophin gene, which causes the synthesis of dystrophin to stop prematurely.

The mutated exon in mdx mice can be removed by targeting it with an antisense oligonucleotide. This results is exon skipping and restores dystrophin expression to levels comparable with those of normal muscle.

mdx mice exhibit phases of marked skeletal muscle degeneration and subsequent regeneration; as the mice age certain muscle types such as the diaphragm show weakness and increased fibrosis.

I. Identification of Non-Steroidal Anti-Inflammatory Compounds

Additional non-steroidal anti-inflammatory compounds can be identified using the mdx mouse model of DMD. For example, mdx mice may be treated with a compound of interest for a period of time (e.g., four weeks, six weeks, eight weeks, three months, four months, five months, six months, etc.) and then tested for a reduction in muscle inflammation, and/or increase in dystrophin. Treatment of mdx mice with compounds that can be used as non-steroidal anti-inflammatory compound of the method described herein will result in the preservation of muscle mass, an increase in dystrophin, and/or improved muscle endurance. Muscle endurance can be assayed by measuring the mean weekly and total running distance based on number of revolutions on a running wheel. Muscle endurance can also be assayed by measuring post-mortem twitch force, titanic force, and specific force generation.

J. Pharmaceutical Compositions and Methods of Treatment

In certain embodiments, the present disclosure provides formulations or compositions suitable for the delivery of antisense oligonucleotides, as described herein. Hence, in certain embodiments, the present disclosure provides pharmaceutically acceptable compositions that comprise an effective amount of an antisense oligonucleotide, formulated together with one or more pharmaceutically acceptable carriers (additives) and/or diluents. While it is possible for the antisense oligonucleotide to be administered alone, in various embodiments, the antisense oligonucleotide is administered as a pharmaceutical formulation (composition). In some embodiments, the antisense oligonucleotide is golodirsen.

In certain embodiments, the present disclosure provides formulations or compositions suitable for the delivery of non-steroidal anti-inflammatory compounds, as described herein.

Hence, in certain embodiments, the present disclosure provides pharmaceutically acceptable compositions that comprise an effective amount of a non-steroidal anti-inflammatory compound, formulated together with one or more pharmaceutically acceptable carriers (additives) and/or diluents. While it is possible for the non-steroidal anti-inflammatory compound to be administered alone, in various embodiments the non-steroidal anti-inflammatory compound is administered as a pharmaceutical formulation (composition). In some embodiments, the non-steroidal anti-inflammatory compound is an NF-κB inhibitor.

The combination therapies of the present disclosure include formulations or compositions suitable for the delivery of antisense oligonucleotides and formulations or compositions suitable for the delivery of non-steroidal anti-inflammatory compounds.

The combination therapies of the present disclosure may be administered alone or with another therapeutic. The additional therapeutic may be administered prior, concurrently or subsequently to the administration of the combination therapy of the present disclosure. For example, the combination therapies of the disclosure may be administered with a steroid and/or an antibiotic. In certain embodiments, the combination therapies of the disclosure are administered to a patient that is on background steroid therapy (e.g., intermittent or chronic/continuous background steroid therapy). One of skill in the art would appreciate that such patients are those who are subject to ongoing, chronic use of steroids (or corticosteroids) on top of which another treatment, such as the combination therapies of the present disclosure, are administered. For example, in some embodiments the patient has been treated with a corticosteroid (e.g., a stable dose of a corticosteroid for four to six, seven, eight, nine, 10, 11, 12, 13, 14, 15, 16, 17, 18, 19, 20, 21, 22, 23, or 24 or more weeks) prior to administration of the combination therapy and continues to receive the steroid therapy. The steroid may be a glucocorticoid or prednisone. Glucocorticoids such as cortisol control carbohydrate, fat and protein metabolism, and are anti-inflammatory by preventing phospholipid release, decreasing eosinophil action and a number of other mechanisms. Mineralocorticoids such as aldosterone control electrolyte and water levels, mainly by promoting sodium retention in the kidney. Corticosteroids are a class of chemicals that includes steroid hormones naturally produced in the adrenal cortex of vertebrates and analogues of these hormones that are synthesized in laboratories. Corticosteroids are involved in a wide range of physiological processes, including stress response, immune response, and regulation of inflammation, carbohydrate metabolism, protein catabolism, blood electrolyte levels, and behavior. Corticosteroids include, but are not limited to, Betamethasone, Budesonide, Cortisone, Dexamethasone, Hydrocortisone, Methylprednisolone, Prednisolone, and Prednisone. One particular steroid of interest that may be administered prior, concurrently or subsequently to the administration of the composition of the present disclosure is deflazacort and formulations thereof (e.g., MP-104, Marathon Pharmaceuticals LLC).

In some embodiments, treatment of patients with the combination therapy may lower the amount of a steroid co-therapy required to maintain a similar level, the same, or even better efficacy than that achieved on a higher dose of the steroid and in the absence of the combination therapy. In some embodiments, patients may be administered dosages of a steroid, such as deflazacort or prednisone, that is at least 5 (e.g., at least 6, 7, 8, 9, 10, 11, 12, 13, 14, 15, 16, 17, 18, 19, 20, 21, 22, 23, 24, 25, 26, 27, 28, 29, 30, 32, 35, 37, 40, 45, 50, 55, 60, 65, or 70) % less than the recommended dose (e.g., as recommended by the CDC/TREAT-NMD guidelines; see, Bushby K, Lynn S, Straub V. Collaborating to bring new therapies to the patient: the TREAT-NMD model. Acta Myo 2009;28:12-15) of steroid for a patient of similar level of disease state or progression. In some embodiments, combination therapy-treated patients are administered between about 75% to about 80% of the recommended dose of a given steroid.

According to the guidelines, the recommended starting dose of prednisone is 0.75 mg/kg/day and that of deflazacort is 0.9 mg/kg/day, given in the morning. Some children experience short-lived behavioral side effects (hyperactivity, mood swings) for a few hours after the medication is given. For these children, administration of the medication in the afternoon may alleviate some of these difficulties. For ambulatory individuals, the dosage is commonly increased as the child grows until he reaches approximately 40 kg in weight. The maximum dose of prednisone is usually capped at approximately 30 mg/day, and that of deflazacort at 36 mg/day. Non-ambulatory teenagers maintained on long-term steroid therapy are usually above 40 kg in weight and the prednisone dosage per kg is often allowed to drift down to the 0.3 to 0.6 mg/kg/day range. While this dosage is less than the approximate 30 mg cap, it demonstrates substantial benefit.

Deciding on a maintenance dose of steroid is a balance between growth of the patient, patient response to steroid therapy, and the burden of side effects. This decision needs to be reviewed at every clinic visit based on the result of the tests done and whether or not side effects are a problem that cannot be managed or tolerated. In DMD patients on a relatively low dosage of steroid (less than the starting dose per kg body weight) who start to show functional decline, it may be necessary to consider a “functional rescue” adjustment. In this situation, the dosage of steroid is increased to the target and the patient is then reevaluated for any benefit in approximately two to three months.

Other agents which can be administered include an antagonist of the ryanodine receptor, such as dantrolene, which has been shown to enhance antisense-mediated exon skipping in patient cells and a mouse model of DMD (G. Kendall et al. Sci Tranl Med 4:164-160 (2012), incorporated herein by reference).

Methods for the delivery of nucleic acid molecules are described, for example, in Akhtar et al., 1992, Trends Cell Bio., 2:139; and Delivery Strategies for Antisense Oligonucleotide Therapeutics, ed. Akhtar; Sullivan et al., PCT WO 94/02595. These and other protocols can be utilized for the delivery of virtually any nucleic acid molecule, including antisense oligonucleotides, e.g., golodirsen.

As detailed below, the pharmaceutical compositions of the present disclosure may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient.

Some examples of materials that can serve as pharmaceutically-acceptable carriers include, without limitation: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

Additional non-limiting examples of agents suitable for formulation with the compound and oligonucleotides of the disclosure include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues; biodegradable polymers, such as poly (DL-lactide-coglycolide) microspheres for sustained release delivery after implantation (Emerich, D F et al., 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999).

The disclosure also features the use of the composition comprising surface-modified liposomes containing poly (ethylene glycol) lipids (PEG-modified, branched and unbranched or combinations thereof, or long-circulating liposomes or stealth liposomes). Antisense oligonucleotides can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

In a further embodiment, the present disclosure includes antisense oligonucleotides, e.g., antisense oligonucleotides that specifically hybridizes to an exon 53 target region of the Dystrophin pre-mRNA and induces exon 53 skipping such as, for example, golodirsen, prepared for delivery as described in U.S. Pat. Nos. 6,692,911, 7,163,695 and 7,070,807. In this regard, in some embodiments, the present disclosure provides antisense oligonucleotides in a composition comprising copolymers of lysine and histidine (HK) (as described in U.S. Pat. Nos. 7,163,695, 7,070,807, and 6,692,911) either alone or in combination with PEG (e.g., branched or unbranched PEG or a mixture of both), in combination with PEG and a targeting moiety or any of the foregoing in combination with a crosslinking agent. In certain embodiments, the present disclosure provides antisense oligonucleotides in a composition comprising gluconic-acid-modified polyhistidine or gluconylated-polyhistidine/transferrin-polylysine. One skilled in the art will also recognize that amino acids with properties similar to His and Lys may be substituted within the composition.

Certain embodiments of antisense oligonucleotides and non-steroidal anti-inflammatory compounds may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present disclosure. These salts can be prepared in situ in the administration vehicle or the dosage form manufacturing process, or by separately reacting a purified compound of the disclosure in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The pharmaceutically acceptable salts of antisense oligonucleotides and/or non-steroidal anti-inflammatory compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In certain embodiments, the antisense oligonucleotides and/or non-steroidal anti-inflammatory compounds may contain one or more acidic functional groups and, thus, is capable of forming pharmaceutically-acceptable salts with pharmaceutically-acceptable bases. The term “pharmaceutically-acceptable salts” in these instances refers to the relatively non-toxic, inorganic and organic base addition salts of compounds of the present disclosure. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, e.g., Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present disclosure include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient that can be combined with a carrier material to produce a single dosage form will vary depending upon the host being treated, the particular mode of administration. The amount of active ingredient which can be combined with a carrier material to produce a single dosage form will generally be that amount of the compound which produces an effect. Generally, out of one hundred percent, this amount will range from about 0.1 percent to about ninety-nine percent of active ingredient. In some embodiments, this amount will range from about 5 percent to about 70 percent, or from about 10 percent to about 30 percent.

In certain embodiments, a formulation of the present disclosure comprises an excipient selected from cyclodextrins, celluloses, liposomes, micelle forming agents, e.g., bile acids, and polymeric carriers, e.g., polyesters and polyanhydrides; and the antisense oligonucleotide and/or non-steroidal anti-inflammatory compound. In certain embodiments, an aforementioned formulation renders orally bioavailable antisense oligonucleotide and/or non-steroidal anti-inflammatory compound.

Methods of preparing these formulations or compositions include the step of bringing into association the antisense oligonucleotide and/or non-steroidal anti-inflammatory compound with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Formulations of the disclosure suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. The antisense oligonucleotide and/or non-steroidal anti-inflammatory compound may also be administered as a bolus, electuary or paste.

In solid dosage forms of the disclosure for oral administration (capsules, tablets, pills, dragees, powders, granules, trouches and the like), the active ingredient may be mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (e.g., gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more compounds of the disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations or dosage forms for the topical or transdermal administration of an oligomer as provided herein include powders, sprays, ointments, pastes, creams, lotions, gels, solutions, patches and inhalants. The antisense oligonucleotide and/or non-steroidal anti-inflammatory compound may be mixed under sterile conditions with a pharmaceutically-acceptable carrier, and with any preservatives, buffers, or propellants which may be required. The ointments, pastes, creams and gels may contain, in addition to an active compound of this disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the antisense oligonucleotide and/or non-steroidal anti-inflammatory compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of an oligomer of the present disclosure to the body. Such dosage forms can be made by dissolving or dispersing the oligomer in the proper medium. Absorption enhancers can also be used to increase the flux of the agent across the skin. The rate of such flux can be controlled by either providing a rate controlling membrane or dispersing the agent in a polymer matrix or gel, among other methods known in the art.

Pharmaceutical compositions suitable for parenteral administration may comprise the antisense oligonucleotide and/or non-steroidal anti-inflammatory compound with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the antisense oligonucleotide and/or non-steroidal anti-inflammatory compound may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility, among other methods known in the art. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms may be made by forming microencapsule matrices of antisense oligonucleotide and/or non-steroidal anti-inflammatory compound in biodegradable polymers such as polylactide-polyglycolide. Depending on the ratio of the antisense oligonucleotide and/or non-steroidal anti-inflammatory compound to polymer, and the nature of the particular polymer employed, the rate of the antisense oligonucleotide and/or non-steroidal anti-inflammatory compound release can be controlled. Examples of other biodegradable polymers include poly(orthoesters) and poly(anhydrides). Depot injectable formulations may also prepared by entrapping the drug in liposomes or microemulsions that are compatible with body tissues.

When the antisense oligonucleotide and/or non-steroidal anti-inflammatory compound is administered as a pharmaceutical, to humans and animals, it can be given per se or as a pharmaceutical composition containing, for example, 0.1 to 99% or 10 to 30%, of active ingredient with a pharmaceutically acceptable carrier.

As noted above, the formulations or preparations of the present disclosure may be given orally, parenterally, systemically, topically, rectally or intramuscular administration. They are typically given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

Regardless of the route of administration selected, the antisense oligonucleotide and/or non-steroidal anti-inflammatory compound, which may be used in a suitable hydrated form, and/or the pharmaceutical compositions of the present disclosure, may be formulated into pharmaceutically-acceptable dosage forms by conventional methods known to those of skill in the art. Actual dosage levels of the active ingredients in the pharmaceutical compositions of this disclosure may be varied so as to obtain an amount of the active ingredient which is effective to achieve the desired response for a particular patient, composition, and mode of administration, without being unacceptably toxic to the patient.

The pharmaceutical compositions of the disclosure may be given by chronic administration to the patient for the treatment of muscular dystrophy. For example, the pharmaceutical compositions may be administered daily, for a period of time of at least several weeks or months or years, or weekly, for a period of time of at least several months or years (e.g., weekly for at least six weeks, weekly for at least 12 weeks, weekly for at least 24 weeks, weekly for at least 48 weeks, weekly for at least 72 weeks, weekly for at least 96 weeks, weekly for at least 120 weeks, weekly for at least 144 weeks, weekly for at least 168 weeks, weekly for at least 180 weeks, weekly for at least 192 weeks, weekly for at least 216 weeks, or weekly for at least 240 weeks).

Alternatively, the pharmaceutical compositions of the disclosure may be given by periodic administration with an interval between doses. For example, the pharmaceutical compositions may be administered at fixed intervals (e.g., weekly, monthly) that may be recurring.

The selected dosage level will depend upon a variety of factors including the activity of the antisense oligonucleotide and/or non-steroidal anti-inflammatory compound, or the ester, salt or amide thereof, the route of administration, the time of administration, the rate of excretion or metabolism of the antisense oligonucleotide and/or non-steroidal anti-inflammatory compound, the rate and extent of absorption, the duration of the treatment, other drugs, compounds and/or materials used with the antisense oligonucleotide and/or non-steroidal anti-inflammatory compound, the age, sex, weight, condition, general health and prior medical history of the patient being treated, and like factors well known in the medical arts. Combination therapies provided herein involve administration of DMD exon-skipping antisense oligonucleotides and anti-inflammatory compounds, to treat subjects afflicted with Duchenne's Muscular Dystrophy (DMD). In some embodiments, the disclosure provides administration of an exon-skipping antisense oligonucleotide and a NF-κB inhibitor to treat subjects having DMD. In some embodiments, the NF-κB inhibitor is CAT-1004 or CAT-1041. In certain embodiments, the exon-skipping antisense oligonucleotide is golodirsen.

In some embodiments, the disclosure provides administration of an exon-skipping antisense oligonucleotide and a NF-κB inhibitor to induce or increase dystrophin protein production in subjects with DMD. In some embodiments, the NF-κB inhibitor is CAT-1004 or CAT-1041. In certain embodiments, the exon-skipping antisense oligonucleotide is golodirsen.

In some embodiments, golodirsen is administered at a dose of 30 mg/kg weekly.

In some embodiments, golodirsen is administered weekly for at least 12 weeks.

In various embodiments, CAT-1004 is administered at a dose of about 33 mg/kg/day, about 67 mg/kg/day, or about 100 mg/kg/day. In some embodiments, CAT-1004 is administered at a dose of about 33 mg/kg, about 67 mg/kg, about 100 mg/kg, about, 125 mg/kg, about 150 mg/kg, about 175 mg/kg, about 200 mg/kg. In some embodiments, CAT-1004 is administered at a dose of about 1 g/day, 2 g/day, 4 g/day, 6 g/day, 8 g/day, and 10 g/day.

In various embodiments, CAT-1004 is administered at a dose of 300 mg, 1000 mg, 2000 mg, 4000 mg, or 6000 mg. In some embodiments, CAT-1004 is administered daily. For example, CAT-1004 may be administered daily for at least 14 days, 1 month, 3 months, 6 months, 9 months, 12 months.

In certain embodiments, the non-steroidal anti-inflammatory compound is administered for at least 12 weeks. In certain embodiments, the non-steroidal anti-inflammatory compound is administered for at least 36 weeks.

In various embodiments, the non-steroidal anti-inflammatory compound is administered prior to, in conjunction with, or subsequent to administration of golodirsen. In some embodiments, golodirsen and the non-steroidal anti-inflammatory compound are administered simultaneously. In some embodiments, golodirsen and the non-steroidal anti-inflammatory compound are administered sequentially. In certain embodiments, golodirsenis administered prior to administration of the non-steroidal anti-inflammatory compound. In various embodiments, the non-steroidal anti-inflammatory compound is administered prior to administration of golodirsen.

In some embodiments, golodirsen is administered intravenously. In some embodiments, golodirsen is administered as an intravenous infusion over 35 to 60 minutes.

In some embodiments, the non-steroidal anti-inflammatory compound is administered orally. In some embodiments, CAT-1004 is formulated for oral delivery, for example, in capsules, as described in U.S. Patent No. 8,173,831, incorporated herein by reference.

In various embodiments, the patient is seven years of age or older. In certain embodiments, the patient is between about 6 months and about 4 years of age. In some embodiments, the patient is between about 4 years of age and 7 years of age.

In some embodiments, combination treatment with golodirsen and a non-steroidal anti-inflammatory compound induces or increases novel dystrophin production, delays disease progression, slows or reduces the loss of ambulation, reduces muscle inflammation, reduces muscle damage, improves muscle function, reduces loss of pulmonary function, and/or enhances muscle regeneration, and any combination thereof. In some embodiments, treatment maintains, delays, or slows disease progression. In some embodiments, treatment maintains ambulation or reduces the loss of ambulation. In some embodiments, treatment maintains pulmonary function or reduces loss of pulmonary function. In some embodiments, treatment maintains or increases a stable walking distance in a patient, as measured by, for example, the 6 Minute Walk Test (6MWT). In some embodiments, treatment maintains, improves, or reduces the time to walk/run 10 meters (i.e., the 10 meter walk/run test). In some embodiments, treatment maintains, improves, or reduces the time to stand from supine (i.e, time to stand test). In some embodiments, treatment maintains, improves, or reduces the time to climb four standard stairs (i.e., the four-stair climb test). In some embodiments, treatment maintains, improves, or reduces muscle inflammation in the patient, as measured by, for example, Mill (e.g., Mill of the leg muscles). In some embodiments, Mill measures a change in the lower leg muscles. In some embodiments, Mill measures T2 and/or fat fraction to identify muscle degeneration. Mill can identify changes in muscle structure and composition caused by inflammation, edema, muscle damage and fat infiltration. In some embodiments, muscle strength is measured by the North Star Ambulatory Assessment. In some embodiments, muscle strength is measured by the pediatric outcomes data collection instrument (PODCI).

In some embodiments, combination treatment with golodirsen and a non-steroidal anti-inflammatory compound of the disclosure reduces muscle inflammation, reduces muscle damage, improves muscle function, and/or enhances muscle regeneration. For example, treatment may stabilize, maintain, improve, or reduce inflammation in the subject. Treatment may also, for example, stabilize, maintain, improve, or reduce muscle damage in the subject. Treatment may, for example, stabilize, maintain, or improve muscle function in the subject. In addition, for example, treatment may stabilize, maintain, improve, or enhance muscle regeneration in the subject. In some embodiments, treatment maintains, improves, or reduces muscle inflammation in the patient, as measured by, for example, magnetic resonance imaging (Mill) (e.g., Mill of the leg muscles) that would be expected without treatment.

In some embodiments, treatment is measured by the 6 Minute Walk Test (6MWT). In some embodiments, treatment is measured by the 10 Meter Walk/Run Test. In various embodiments, the treatment results in a reduction or decrease in muscle inflammation in the patient. In certain embodiments, muscle inflammation in the patient is measured by MRI imaging. In some embodiments, the treatment is measured by the 4-stair climb test. In various embodiments, treatment is measured by the time to stand test. In some embodiments, treatment is measured by the North Star Ambulatory Assessment.

In some embodiments, the method of the disclosure further comprises administering to the patient a corticosteroid. In certain embodiments, the corticosteroid is Betamethasone, Budesonide, Cortisone, Dexamethasone, Hydrocortisone, Methylprednisolone, Prednisolone, Prednisone, or Deflazacort. in various embodiments, the corticosteroid is administered prior to, in conjunction with, or subsequent to administration of golodirsen.

In some embodiments, the method of the disclosure further comprises confirming that the patient has a mutation in the DMD gene that is amenable to exon 53 skipping. In certain embodiments, the method of the disclosure further comprises confirming that the patient has a mutation in the DMD gene that is amenable to exon 53 skipping prior to administering golodirsen.

In some embodiments, the patient has lost the ability to rise independently from supine. In some embodiments, the patient loses the ability to rise independently from supine at least one year prior to treatment with golodirsen. In various embodiments, the patient loses the ability to rise independently from supine within one year of commencing treatment with golodirsen. In certain embodiments, the patient loses the ability to rise independently from supine within two years of commencing treatment with golodirsen.

In some embodiments, the patient maintains ambulation for at least 24 weeks after commencing treatment with golodirsen. In certain embodiments, the patient has a reduction in the loss of ambulation for at least 24 weeks immediately after commencing treatment with golodirsen as compared to a placebo control.

In some embodiments, dystrophin protein production is measured by reverse transcription polymerase chain reaction (RT-PCR), western blot analysis, or immunohistochemistry (IHC).

In some embodiments, the dosage of the antisense oligonucleotide (e.g., golodirsen) is about 30 mg/kg over a period of time sufficient to treat DMD or BMD. In some embodiments, the antisense oligonucleotide is administered to the patient at a dose of between about 25 mg/kg and about 50 mg/kg (e.g., about 25, 26, 27, 28, 29, 30, 31, 32, 33, 34, 35, 36, 37, 38, 39, 40, 41, 42, 43, 44, 45, 46, 47, 48, 49, or 50 mg/kg), e.g., once per week. In some embodiments, the antisense oligonucleotide is administered to the patient at a dose of between about 25 mg/kg and about 50 mg/kg (e.g., about 30 mg/kg to about 50 mg/kg, about 25 mg/kg to about 40 mg/kg, about 28 mg/kg to about 32 mg/kg, or about 30 mg/kg to about 40 mg/kg), e.g., once per week.

In some embodiments, the antisense compound for inducing exon skipping in the human dystrophin pre-mRNA is administered at a lower dose and/or for shorter durations and/or reduced frequency than prior approaches when used as a combination therapy with a non-steroidal anti-inflammatory compound.

In some embodiments, the antisense oligonucleotide is administered intravenously once a week. In certain embodiments, the time of infusion is from about 15 minutes to about 4 hours. In some embodiments, the time of infusion is from about 30 minutes to about 3 hours. In some embodiments, the time of infusion is from about 30 minutes to about 2 hours. In some embodiments, the time of infusion is from about 1 hour to about 2 hours. In some embodiments the time of infusion is from about 30 minutes to about 1 hour. In some embodiments, the time of infusion is about 60 minutes. In some embodiments, the time of infusion is 35 to 60 minutes.

In some embodiments, the dosage on the non-steroidal anti-inflammatory compound (e.g., an NF-κB inhibitor (e.g., CAT-1004)) is about 33 mg/kg, 67 mg/kg, or 100 mg/kg. In some embodiments, the non-steroidal anti-inflammatory compound is administered to the patient at a dose of between about 10 mg/kg and about 1000 mg/kg (e.g., about 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, 500, 550, 600, 650, 700, 750, 800, 850, 900, or 1000 mg/kg), e.g, once per day, twice per day, three times per day, once every other day once per week, biweekly, once per month, or bimonthly. In some embodiments, an effective amount is about 10 mg/kg to about 50 mg/kg, or about 10 mg/kg to about 100 mg/kg, or about 50 mg/kg to about 100 mg/kg, or about 50 mg/kg to about 200 mg/kg, or about 100 mg/kg to about 300 mg/kg, or about 100 mg/kg to about 500 mg/kg, or about 200 mg/kg to about 600 mg/kg, or about 500 mg/kg to about 800 mg/kg, or about 500 mg/kg to about 1000 mg/kg, once per day, twice per day, three times per day, once every other day, once per week, biweekly, once per month, or bimonthly.

Alternatively, dosages may be given in absolute terms, for example, 10 mg, 20 mg, 30 mg, 40 mg, 50 mg, 60 mg, 70 mg, 80 mg, 80 mg, 100 mg, 110 mg, 120 mg, 130 mg, 140 mg, 150 mg, 160 mg, 170 mg, 180 mg, 190 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1500 mg, 2000 mg, 2500 mg, 3000 mg, 3500 mg, 4000 mg, 4500 mg, 5000 mg, 5500 mg, 6000 mg, 6500 mg, 7000 mg, 7500 mg, 8000 mg, 8500 mg, 9000 mg, 9500 mg, or 10,000 mg. The compound may be administered over a period of days, weeks, months, or years.

In some embodiments, the non-steroidal anti-inflammatory compound is administered orally once per day, twice per day, three times per day, once per week, biweekly, once per month, or bimonthly.

The non-steroidal anti-inflammatory compound can be formulated for oral administration, for example, in a tablet or gel cap. Formulations comprising the compounds can be taken with food or in a fasting state. When the formulation is taken with food, the food content may be adjusted to facilitate absorption of the active compound. For example, the formulation may be taken with low-fat or high-fat meals. The formulation can be administered as a single dose or in multiple periodic doses, for example, one, two, or three doses per day. Dosage of the active compound may be adjusted based on the size of the subject.

Administration of the combination therapy (antisense oligonucleotide and non-steroidal anti-inflammatory compound) may be followed by, or concurrent with, administration of an antibiotic, steroid or other agent. The treatment regimen may be adjusted (dose, frequency, route, etc.) as indicated, based on the results of immunoassays, other biochemical tests and physiological examination of the subject under treatment.

Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres, as described herein and known in the art. In certain embodiments, microemulsification technology may be utilized to improve bioavailability of lipophilic (water insoluble) pharmaceutical agents. Examples include Trimetrine (Dordunoo, S. K., et al., Drug Development and Industrial Pharmacy, 17(12), 1685-1713, 1991 and REV 5901 (Sheen, P. C., et al., J Pharm Sci 80(7), 712-714, 1991). Among other benefits, microemulsification provides enhanced bioavailability by preferentially directing absorption to the lymphatic system instead of the circulatory system, which thereby bypasses the liver, and prevents destruction of the compounds in the hepatobiliary circulation.

In one aspect of disclosure, the formulations contain micelles formed from the antisense oligonucleotide and/or non-steroidal anti-inflammatory compound and at least one amphiphilic carrier, in which the micelles have an average diameter of less than about 100 nm. Various embodiments provide micelles having an average diameter less than about 50 nm, and certain embodiments provide micelles having an average diameter less than about 30 nm, or even less than about 20 nm.

While all suitable amphiphilic carriers are contemplated, in various embodiments carriers are generally those that have Generally-Recognized-as-Safe (GRAS) status, and that can both solubilize the compound of the present disclosure and microemulsify it at a later stage when the solution comes into a contact with a complex water phase (such as one found in human gastro-intestinal tract). Usually, amphiphilic ingredients that satisfy these requirements have HLB (hydrophilic to lipophilic balance) values of 2-20, and their structures contain straight chain aliphatic radicals in the range of C-6 to C-20. Examples are polyethylene-glycolized fatty glycerides and polyethylene glycols.

Examples of amphiphilic carriers include saturated and monounsaturated polyethyleneglycolyzed fatty acid glycerides, such as those obtained from fully or partially hydrogenated various vegetable oils. Such oils may advantageously consist of tri-, di-, and mono-fatty acid glycerides and di- and mono-polyethyleneglycol esters of the corresponding fatty acids, including, for example, capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series).

Commercially available amphiphilic carriers may be particularly useful, including Gelucire-series, Labrafil, Labrasol, or Lauroglycol (all manufactured and distributed by Gattefosse Corporation, Saint Priest, France), PEG-mono-oleate, PEG-di-oleate, PEG-mono-laurate and di-laurate, Lecithin, Polysorbate 80, etc (produced and distributed by a number of companies in USA and worldwide).

In certain embodiments, the delivery may occur by use of liposomes, nanocapsules, microparticles, microspheres, lipid particles, vesicles, and the like, for the introduction of the compositions of the present disclosure into suitable host cells. In particular, the compositions of the present disclosure may be formulated for delivery either encapsulated in a lipid particle, a liposome, a vesicle, a nanosphere, a nanoparticle or the like. The formulation and use of such delivery vehicles can be carried out using known and conventional techniques.

Hydrophilic polymers suitable for use in the present disclosure are those which are readily water-soluble, can be covalently attached to a vesicle-forming lipid, and which are tolerated in vivo without toxic effects (i.e., are biocompatible). Suitable polymers include polyethylene glycol (PEG), polylactic (also termed polylactide), polyglycolic acid (also termed polyglycolide), a polylactic-polyglycolic acid copolymer, and polyvinyl alcohol. In certain embodiments, polymers have a molecular weight of from about 100 or 120 daltons up to about 5,000 or 10,000 daltons, or from about 300 daltons to about 5,000 daltons. In other embodiments, the polymer is polyethyleneglycol having a molecular weight of from about 100 to about 5,000 daltons, or having a molecular weight of from about 300 to about 5,000 daltons. In certain embodiments, the polymer is polyethyleneglycol of 750 daltons (PEG(750)). Polymers may also be defined by the number of monomers therein; various embodiments of the present disclosure utilizes polymers of at least about three monomers, such PEG polymers consisting of three monomers (approximately 150 daltons).

Other hydrophilic polymers which may be suitable for use in the present disclosure include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In certain embodiments, a formulation of the present disclosure comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

Cyclodextrins are cyclic oligosaccharides, consisting of 6, 7 or 8 glucose units, designated by the Greek letter α, β, or γ, respectively. The glucose units are linked by α-1,4-glucosidic bonds. As a consequence of the chair conformation of the sugar units, all secondary hydroxyl groups (at C-2, C-3) are located on one side of the ring, while all the primary hydroxyl groups at C-6 are situated on the other side. As a result, the external faces are hydrophilic, making the cyclodextrins water-soluble. In contrast, the cavities of the cyclodextrins are hydrophobic, since they are lined by the hydrogen of atoms C-3 and C-5, and by ether-like oxygens. These matrices allow complexation with a variety of relatively hydrophobic compounds, including, for instance, steroid compounds such as 17α-estradiol (see, e.g., van Uden et al. Plant Cell Tiss. Org. Cult. 38:1-3-113 (1994)). The complexation takes place by Van der Waals interactions and by hydrogen bond formation. For a general review of the chemistry of cyclodextrins, see, Wenz, Agnew. Chem. Int. Ed. Engl., 33:803-822 (1994).

The physico-chemical properties of the cyclodextrin derivatives depend strongly on the kind and the degree of substitution. For example, their solubility in water ranges from insoluble (e.g., triacetyl-beta-cyclodextrin) to 147% soluble (w/v) (G-2-beta-cyclodextrin). In addition, they are soluble in many organic solvents. The properties of the cyclodextrins enable the control over solubility of various formulation components by increasing or decreasing their solubility.

Numerous cyclodextrins and methods for their preparation have been described. For example, Parmeter (I), et al. (U.S. Pat. No. 3,453,259) and Gramera, et al. (U.S. Pat. No. 3,459,731) described electroneutral cyclodextrins. Other derivatives include cyclodextrins with cationic properties [Parmeter (II), U.S. Pat. No. 3,453,257], insoluble crosslinked cyclodextrins (Solms, U.S. Pat. No. 3,420,788), and cyclodextrins with anionic properties [Parmeter (III), U.S. Pat. No. 3,426,011]. Among the cyclodextrin derivatives with anionic properties, carboxylic acids, phosphorous acids, phosphinous acids, phosphonic acids, phosphoric acids, thiophosphonic acids, thiosulphinic acids, and sulfonic acids have been appended to the parent cyclodextrin [see, Parmeter (III), supra]. Furthermore, sulfoalkyl ether cyclodextrin derivatives have been described by Stella, et al. (U.S. Pat. No. 5,134,127).

Liposomes consist of at least one lipid bilayer membrane enclosing an aqueous internal compartment. Liposomes may be characterized by membrane type and by size. Small unilamellar vesicles (SUVs) have a single membrane and typically range between 0.02 and 0.05 μm in diameter; large unilamellar vesicles (LUVS) are typically larger than 0.05 μm. Oligolamellar large vesicles and multilamellar vesicles have multiple, usually concentric, membrane layers and are typically larger than 0.1 μm. Liposomes with several nonconcentric membranes, i.e., several smaller vesicles contained within a larger vesicle, are termed multivesicular vesicles.

One aspect of the present disclosure relates to formulations comprising liposomes containing the antisense oligonucleotide (e.g., golodirsen) and/or the non-steroidal anti-inflammatory compound, where the liposome membrane is formulated to provide a liposome with increased carrying capacity. Alternatively or in addition, the compound of the present disclosure may be contained within, or adsorbed onto, the liposome bilayer of the liposome. The antisense oligonucleotide and/or non-steroidal anti-inflammatory compound may be aggregated with a lipid surfactant and carried within the liposome's internal space; in these cases, the liposome membrane is formulated to resist the disruptive effects of the active agent-surfactant aggregate.

According to some embodiments of the present disclosure, the lipid bilayer of a liposome contains lipids derivatized with polyethylene glycol (PEG), such that the PEG chains extend from the inner surface of the lipid bilayer into the interior space encapsulated by the liposome, and extend from the exterior of the lipid bilayer into the surrounding environment.

Active agents contained within liposomes of the present disclosure are in solubilized form. Aggregates of surfactant and active agent (such as emulsions or micelles containing the active agent of interest) may be entrapped within the interior space of liposomes according to the present disclosure. A surfactant acts to disperse and solubilize the active agent, and may be selected from any suitable aliphatic, cycloaliphatic or aromatic surfactant, including but not limited to biocompatible lysophosphatidylcholines (LPGs) of varying chain lengths (for example, from about C14 to about C20). Polymer-derivatized lipids such as PEG-lipids may also be utilized for micelle formation as they will act to inhibit micelle/membrane fusion, and as the addition of a polymer to surfactant molecules decreases the CMC of the surfactant and aids in micelle formation. Some embodiments, for example, include surfactants with CMOs in the micromolar range; higher CMC surfactants may be utilized to prepare micelles entrapped within liposomes of the present disclosure.

Liposomes according to the present disclosure may be prepared by any of a variety of techniques that are known in the art. See, e.g., U.S. Pat. No. 4,235,871; Published PCT applications WO 96/14057; New RRC, Liposomes: A practical approach, IRL Press, Oxford (1990), pages 33-104; Lasic DD, Liposomes from physics to applications, Elsevier Science Publishers BV, Amsterdam, 1993. For example, liposomes of the present disclosure may be prepared by diffusing a lipid derivatized with a hydrophilic polymer into preformed liposomes, such as by exposing preformed liposomes to micelles composed of lipid-grafted polymers, at lipid concentrations corresponding to the final mole percent of derivatized lipid which is desired in the liposome. Liposomes containing a hydrophilic polymer can also be formed by homogenization, lipid-field hydration, or extrusion techniques, as are known in the art.

In another exemplary formulation procedure, the active agent is first dispersed by sonication in a lysophosphatidylcholine or other low CMC surfactant (including polymer grafted lipids) that readily solubilizes hydrophobic molecules. The resulting micellar suspension of active agent is then used to rehydrate a dried lipid sample that contains a suitable mole percent of polymer-grafted lipid, or cholesterol. The lipid and active agent suspension is then formed into liposomes using extrusion techniques as are known in the art, and the resulting liposomes separated from the unencapsulated solution by standard column separation.

In one aspect of the present disclosure, the liposomes are prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. See e.g., U.S. Pat. No. 4,737,323 (Apr. 12, 1988). In certain embodiments, reagents such as DharmaFECT® and Lipofectamine® may be utilized to introduce polynucleotides or proteins into cells.

The release characteristics of a formulation of the present disclosure depend on the encapsulating material, the concentration of encapsulated drug, and the presence of release modifiers. For example, release can be manipulated to be pH dependent, for example, using a pH sensitive coating that releases only at a low pH, as in the stomach, or a higher pH, as in the intestine. An enteric coating can be used to prevent release from occurring until after passage through the stomach. Multiple coatings or mixtures of cyanamide encapsulated in different materials can be used to obtain an initial release in the stomach, followed by later release in the intestine. Release can also be manipulated by inclusion of salts or pore forming agents, which can increase water uptake or release of drug by diffusion from the capsule. Excipients which modify the solubility of the drug can also be used to control the release rate. Agents which enhance degradation of the matrix or release from the matrix can also be incorporated. They can be added to the drug, added as a separate phase (i.e., as particulates), or can be co-dissolved in the polymer phase depending on the compound. In most cases the amount should be between 0.1 and thirty percent (w/w polymer). Types of degradation enhancers include inorganic salts such as ammonium sulfate and ammonium chloride, organic acids such as citric acid, benzoic acid, and ascorbic acid, inorganic bases such as sodium carbonate, potassium carbonate, calcium carbonate, zinc carbonate, and zinc hydroxide, and organic bases such as protamine sulfate, spermine, choline, ethanolamine, diethanolamine, and triethanolamine and surfactants such as Tween® and Pluronic®. Pore forming agents which add microstructure to the matrices (i.e., water soluble compounds such as inorganic salts and sugars) are added as particulates. The range is typically between one and thirty percent (w/w polymer).

Uptake can also be manipulated by altering residence time of the particles in the gut. This can be achieved, for example, by coating the particle with, or selecting as the encapsulating material, a mucosal adhesive polymer. Examples include most polymers with free carboxyl groups, such as chitosan, celluloses, and especially polyacrylates (as used herein, polyacrylates refers to polymers including acrylate groups and modified acrylate groups such as cyanoacrylates and methacrylates).

The antisense oligonucleotide and/or non-steroidal anti-inflammatory compound may be formulated to be contained within, or, adapted to release by a surgical or medical device or implant. In certain aspects, an implant may be coated or otherwise treated with the antisense oligonucleotide and/or non-steroidal anti-inflammatory compound. For example, hydrogels, or other polymers, such as biocompatible and/or biodegradable polymers, may be used to coat an implant with the compositions of the present disclosure (i.e., the composition may be adapted for use with a medical device by using a hydrogel or other polymer). Polymers and copolymers for coating medical devices with an agent are well-known in the art. Examples of implants include, but are not limited to, stents, drug-eluting stents, sutures, prosthesis, vascular catheters, dialysis catheters, vascular grafts, prosthetic heart valves, cardiac pacemakers, implantable cardioverter defibrillators, IV needles, devices for bone setting and formation, such as pins, screws, plates, and other devices, and artificial tissue matrices for wound healing.

In addition to the methods provided herein, the antisense oligonucleotide and/or non-steroidal anti-inflammatory compound may be formulated for administration in any convenient way for use in human or veterinary medicine, by analogy with other pharmaceuticals. The antisense oligonucleotide and/or non-steroidal anti-inflammatory compound and its corresponding formulation may be administered alone or as a combination therapy with other therapeutic strategies in the treatment of muscular dystrophy, such as myoblast transplantation, stem cell therapies, administration of aminoglycoside antibiotics, proteasome inhibitors, and up-regulation therapies (e.g., upregulation of utrophin, an autosomal paralogue of dystrophin).

The routes of administration described are intended only as a guide since a skilled practitioner will be able to determine readily the optimum route of administration and any dosage for any particular animal and condition. Multiple approaches for introducing functional new genetic material into cells, both in vitro and in vivo have been attempted (Friedmann (1989) Science, 244:1275-1280). These approaches include integration of the gene to be expressed into modified retroviruses (Friedmann (1989) supra; Rosenberg (1991) Cancer Research 51(18), suppl.: 5074S-5079S); integration into non-retrovirus vectors (e.g., adeno-associated viral vectors) (Rosenfeld, et al. (1992) Cell, 68:143-155; Rosenfeld, et al. (1991) Science, 252:431-434); or delivery of a transgene linked to a heterologous promoter-enhancer element via liposomes (Friedmann (1989), supra; Brigham, et al. (1989) Am. J. Med. Sci., 298:278-281; Nabel, et al. (1990) Science, 249:1285-1288; Hazinski, et al. (1991) Am. J. Resp. Cell Molec. Biol., 4:206-209; and Wang and Huang (1987) Proc. Natl. Acad. Sci. (USA), 84:7851-7855); coupled to ligand-specific, cation-based transport systems (Wu and Wu (1988) J. Biol. Chem., 263:14621-14624) or the use of naked DNA, expression vectors (Nabel et al. (1990), supra); Wolff et al. (1990) Science, 247:1465-1468). Direct injection of transgenes into tissue produces only localized expression (Rosenfeld (1992) supra); Rosenfeld et al. (1991) supra; Brigham et al. (1989) supra; Nabel (1990) supra; and Hazinski et al. (1991) supra). The Brigham et al. group (Am. J. Med. Sci. (1989) 298:278-281 and Clinical Research (1991) 39 (abstract)) have reported in vivo transfection only of lungs of mice following either intravenous or intratracheal administration of a DNA liposome complex. An example of a review article of human gene therapy procedures is: Anderson, Science (1992) 256:808-813.

In a further embodiment, pharmaceutical compositions of the disclosure may additionally comprise a carbohydrate as provided in Han et al., Nat. Comms. 7, 10981 (2016) the entirety of which is incorporated herein by reference. In some embodiments, pharmaceutical compositions of the disclosure may comprise 5% of a hexose carbohydrate. For example, pharmaceutical composition of the disclosure may comprise 5% glucose, 5% fructose, or 5% mannose. In certain embodiments, pharmaceutical compositions of the disclosure may comprise 2.5% glucose and 2.5% fructose. In some embodiments, pharmaceutical compositions of the disclosure may comprises a carbohydrate selected from: arabinose present in an amount of 5% by volume, glucose present in an amount of 5% by volume, sorbitol present in an amount of 5% by volume, galactose present in an amount of 5% by volume, fructose present in an amount of 5% by volume, xylitol present in an amount of 5% by volume, mannose present in an amount of 5% by volume, a combination of glucose and fructose each present in an amount of 2.5% by volume, and a combination of glucose present in an amount of 5.7% by volume, fructose present in an amount of 2.86% by volume, and xylitol present in an amount of 1.4% by volume.

K. Kits

The disclosure also provides kits for treatment of a patient with muscular dystrophy (e.g., DMD or BMD) which kit comprises at least an antisense molecule (e.g., one or more antisense oligonucleotides capable of specifically hybridizing to any one or more of exons 1-79 of the dystrophin pre-mRNA, for example, any one of the antisense oligonucleotides set forth as SEQ ID Nos. 1-10and 20 in Table 3 herein), packaged in a suitable container, as well an a non-steroidal anti-inflammatory agent (e.g., an NF-κB inhibitor such as CAT-1004), packaged in a suitable container, together with instructions for its use. The kits may also contain peripheral reagents such as buffers, stabilizers, etc. Those of ordinary skill in the field should appreciate that applications of the above method has wide application for identifying antisense molecules and/or non-steroidal anti-inflammatory compounds suitable for use in the treatment of many other diseases.

In one embodiment, the kit comprises a container comprising edasalonexent, and an optional pharmaceutically acceptable carrier, and a package insert comprising instructions for administration of edasalonexent in combination with golodirsen, an optional pharmaceutically acceptable carrier for treating or delaying progression of DMD in a patient.

In another embodiment, the kit comprises a first container, a second container and a package insert, wherein the first container comprises at least one dose of a medicament comprising golodirsen, the second container comprises at least one dose of a medicament comprising edasalonexent, and the package insert comprises instructions for treating a DMD patient by administration of the medicaments.

In some embodiments, the instructions provide for simultaneous administration of golodirsen and edasalonexent. In some embodiments, the instructions provide for sequential administration of golodirsen and edasalonexent. In some embodiments, the instructions provide for administration of golodirsen prior to administration of edasalonexent. In some embodiments, the instructions provide for administration of edasalonexent prior to administration of golodirsen.

### EXAMPLES

### Materials and Methods

### Preparation of CAT-1004 Feed

A pharmacokinetic dose study of CAT-1004 was performed in mice to determine the concentration of CAT-1004 in the diet that gives an equivalent exposure as CAT-1004 in human. Based on this study a 1% CAT-1004 diet was prepared and stored at either −20° C. or −80° C. The feed was removed from the freezer 24 hours prior to adding it to the mouse cages.

### PMO and CAT-1004 Efficacy Study in Mdx Mice

Wild-type (WT) (C57BL/10ScSn/J) and Mdx (C57BL/10ScSn-Dmd^{mdx}/J) mice were used to test the efficacy of the M23D PMO (AVI-4225) in combination with CAT-1004. 5-week old mice were acquired from Jackson Labs and acclimated for one-week. The treatment duration was 4 weeks and began when the mice were 6 weeks of age. Mice were divided into the following five treatment groups, each with N=12: (1) wild-type mice treated with saline, (2) Mdx mice treated with saline, (3) Mdx mice treated with CAT-1004, (4) Mdx mice treated with the M23D PMO, and (5) Mdx mice treated with the M23D PMO in combination with CAT-1004. Mice were dosed weekly with M23D PMO (AVI-4225) at 40 mg/kg by IV injection and treated with CAT-1004 (1%) in their diet. All non-CAT-1004 animals were fed a normal chow control diet and all non-M23D PMO animals were given weekly IV injections of saline. Food consumption was closely monitored and the feed was changed twice per week. Mice were sacrificed at 10 weeks of age (4 weeks post-first dose). The quadriceps, diaphragm, and heart were harvested from each of the respective treatment groups.

### Exon Skipping, Dystrophin Protein Analysis and Histology

For exon skipping analysis, quadriceps, diaphragm, and heart tissue samples were homogenized. After homogenization, RNA was extracted from each of the tissues using GE RNAspin kits (GE Healthcare Life Sciences CAT No: 25-0500-70). Subsequently, RT-PCR was performed to analyze exon-23 skipping. Exon 23 skipping was determined by Caliper imaging. The expected fragments were 445 bp for non-skipped and 245 bp for skipped. Percentage of skipping was determined using the formula: % skipping=skipped molarity/(unskipped+skipped molarity)×100%.

Dystrophin protein was analyzed by Western blot analysis, and immunohistochemistry. For Western blot analysis, heart, diaphragm and quadriceps tissue samples were shaved using a scalpel and then lysed. Total protein concentration of the protein lysates were measured using Pierce™BCA Protein Assay Kit (ThermoFisher Scientific catalog #23225). 50 ug protein samples were prepared, run on a protein gel via electrophoresis, and transferred to a membrane for Western blotting. The membranes were blocked in 5% nonfat milk for 1 hour at room temperature, and then incubated with 1:1000 anti-dystrophin primary antibody (Abcam, catalog # ab15277) in 5% nonfat milk for 16-18 hours at 4° C. or 2 hours at room temperature, or 1:5000 anti-actinin (Sigma, A7811). After incubation, the membranes were washed and then incubated with 1:10,000 secondary antibodies (goat anti-rabbit HRP-conjugated (BioRad, catalog #1706515) for dystrophin, or goat anti-mouse HRP-conjugated (BioRad, catalog # 1706516) for actinin) for 1 hour at room temperature. The membranes were incubated with Clarity Western ECL Solution (BioRad, catalog #1705061) and then visualized with the ChemiDoc Touch auto-exposure setting.

For immunohistochemistry, frozen quadriceps sections were serially cut and mounted on slides using a cryostat. Sections were rehydrated in PBS and then blocked with Mouse on Mouse (MOM) blocking buffer for 1 hour at room temperature. After the blocking buffer was removed, dystrophin primary antibody (dilution 1:250, rabbit, Abcam, cat #ab15277) and laminin (1:250) was added in an antibody dilution buffer and incubated overnight at 4° C. Primary antibody as removed and the sections were washed prior to incubation with secondary antibody Alexa-Fluoro 488 goat anti-rabbit (1:10000 dilution) for 1-2 hours at room temperature. After washing, the sections were rinsed and placed on glass slides with mounting media with DAPI.

To perform histology studies, serial sections were taken from each of the respective tissues. Hematoxylin and Eosin (H&E) staining as well as picrosirius red staining was performed. Specifically, tissues were fixed in ice-cold acetone for 5 minutes and then rehydrated in descending ethanol solutions. The rehydrated sections were dipped in hematoxylin, rinsed with tap water, dipped in 70% ethanol, and then dipped in eosin. The tissue was then dehydrated, dipped in Xylenes and then mounted on slides in 2:1 permount:xylenes solution. For picrosirius red staining, rehydrated tissues were incubated in pircosirius red solution for one hour at room temperature. The tissue was then rinsed with 0.5% acetic acid and then absolute alcohol, prior to being mounted in 2:1 permount:xylenes solution.

### Example 1

### CAT-1004 in Combination with M23D PMO Reduces Inflammation and Fibrosis in Mdx Mice.

To assess the effectiveness of a combination treatment of an exon skipping antisense oligonucleotide and an NF-Kb inhibitor in Duchenne muscular dystrophy, M23D PMO and CAT-1004 were utilized in the Mdx mouse model. The effect on inflammation and fibrosis was determined by analyzing samples of muscle taken from the quadriceps, of (1) wild-type mice treated with saline, (2) mdx mice treated with saline, (3) mdx mice treated with CAT-1004, (4) mdx mice treated with the M23D PMO, and (5) mdx mice treated with the M23D PMO in combination with CAT-1004. The tissue sections were analyzed for fibrosis by picrosirius red staining and for inflammation and fibrosis by Hematoxylin and Eosin (H&E) staining, as described in the Materials and Methods section above.

Treatment of Mdx mice with either M23D PMO or CAT-1004 as monotherapies resulted in a reduction of inflammation and fibrosis as compared to Mdx mice treated with saline. Surprisingly, treatment of Mdx mice with the M23D PMO in combination with CAT-1004 resulted in reduced inflammation and fibrosis as compared with mice treated with CAT-1004 alone or M23D alone (FIG. 9). These results indicate the combination treatment enhances muscle fiber integrity.

### Example 2

### Exon Skipping and Dystrophin Production in Mdx Mice Treated with the M23D PMO and the M23D PMO in Combination with CAT-1004

To analyze the extent of exon skipping and dystrophin production in mice treated with the M23D PMO in combination with CAT-1004, samples of muscle were taken from the quadriceps, diaphragm, and heart of (1) wild-type mice treated with saline, (2) mdx mice treated with saline, (3) mdx mice treated with CAT-1004, (4) mdx mice treated with the M23D PMO, and (5) mdx mice treated with the M23D PMO in combination with CAT-1004. RT-PCR analysis for exon 23 skipping was performed as well as Western blot analysis to determine dystrophin protein levels.

Exon skipping was observed in the muscle of the quadriceps, diaphragm, and heart of the Mdx mice treated with the M23D PMO as well as mice treated with the M23D PMO in combination with CAT-1004 (FIG. 10). Surprisingly, enhanced dystrophin production was observed in the muscle of the quadriceps, diaphragm, and heart of the mice treated with the M23D PMO in combination with CAT-1004 as compared to treatment with M23D PMO monotherapy (FIG. 11). These results indicated the increase in dystrophin levels extended to the heart, a tissue known to have low efficiency of dystrophin upregulation by these agents when used alone. Notably, neither exon skipping nor dystrophin production were observed in mdx mice treated with CAT-1004 monotherapy (FIGS. 10 and 11).

### Example 3

### Immunohistochemical analysis of Dystrophin Expression in the Quadriceps

To further analyze dystrophin expression, immunohistochemical analysis was performed in sections of muscle taken from the quadriceps of (1) wild-type mice treated with saline, (2) mdx mice treated with saline, (3) mdx mice treated with CAT-1004, (4) mdx mice treated with the M23D PMO, and (5) mdx mice treated with the M23D PMO in combination with CAT-1004.

Tissue sections were stained with both dystrophin and laminin. The results are shown in FIG. 12. An increase in dystrophin expression was observed in Mdx mice treated with the M23D PMO monotherapy as well as the M23D PMO in combination with CAT-1004 as compared to Mdx control mice treated with saline or Mdx mice treated with CAT-1004 monotherapy. These results indicated that combination treatment further enhanced sarcolemmal dystrophin

All publications and patent applications cited in this specification are herein incorporated by reference as if each individual publication or patent application were specifically and individually indicated to be incorporated by reference.

### REFERENCES

American Thoracic Society (ATS). ATS statement: guidelines for the six-minute walk test. Am J Respir Crit Care Med. 2002; 166: 111-117.
Aartsma-Rus A, Fokkema I, Verschuuren J, Ginjaar I, van Deutekom J, van Ommen G J, et al. Theoretic applicability of antisense-mediated exon skipping for Duchenne muscular dystrophy mutations. Human Mutation. 2009; 30(3):293-9.
Anthony K, Cirak S, Torelli S, et al. Dystrophin quantification and clinical correlations in Becker muscular dystrophy: implications for clinical trials. Brain: a journal of neurology. December 2011; 134(Pt 12):3547-3559.
Anthony K, Arechavala-Gomeza V, Taylor L, et al. Dystrophin quantification Biological and translational research implications. Neurology. 2014a Nov 25; 83(22):2062-9.
Anthony K, Arechavala-Gomeza V, Ricotti V, et al. Biochemical characterization of patients with in-frame or out-of-frame DMD deletions pertinent to exon 44 or 45 skipping. JAMA neurology. 2014b; 71(1):32-40.
Arechavala-Gomeza V, Graham I R, Popplewell L J, Adams A M, Aartsma-Rus A, Kinali M, et al. Comparative analysis of antisense oligonucleotide sequences for targeted skipping of exon 51 during dystrophin pre-mRNA splicing in human muscle. Hum Gene Ther 2007; 18:798-810.
Barton-Davis E R, Cordier L, Shoturma D I, Leland S E, Sweeny H L. Aminoglycoside antibiotics restore dystrophin function to skeletal muscles of mdx mice. J Clin Invest. 1999 Aug; 104(4):375-81.
Beenakker E A, Fock J M, Van Tol M J, Maurits N M, Koopman H M, Brouwer O F, et al. Intermittent prednisone therapy in Duchenne muscular dystrophy: a randomized controlled trial. Archives of Neurology. 2005; 62(1):128-32.
Bello L; Morgenroth L P, Gordish-Dressman H, Hoffman E P, McDonald C P, Cirak S, on behalf of the CINRG investigators (2016* pre-publication provided courtesy of Craig McDonald, MD, Study Chair of the CINRG Duchenne Natural History Study)
Bello L, Gordish-Dressman H, Morgenroth L P, Henricson E K, Duong T, Hoffman E P, Cnaan A, McDonald C M; CINRG Investigators. Prednisone/prednisolone and deflazacort regimens in the CINRG Duchenne Natural History Study. Neurology. 2015 September; 85(12):1048-55.
Bendixen R M, Senesac C, Lott D J, Vandenborne K. Participation and quality of life in children with Duchenne muscular dystrophy using the International Classification of Functioning, Disability, and Health. Health and Quality of Life Outcomes. 2012; 10:43.
Bendixen R M, Lott D J, Senesac C, Mathur S, Vandenborne K. Participation in daily life activities and its relationship to strength and functional measures in boys with Duchenne muscular dystrophy. Disability and Rehabilitation. 2014; 36(22):1918-23.
Biggar W D, Gingras M, Fehlings D L, Harris V A, Steele C A. Deflazacort treatment of Duchenne muscular dystrophy. The Journal of pediatrics. 2001; 138(1):45-50.
Biggar W D, Harris V A, Eliasoph L, Alman B. Long-term benefits of deflazacort treatment for boys with Duchenne muscular dystrophy in their second decade. Neuromuscular Disorders. 2006; 16(4):249-55.
Bovolenta M, Scotton C, Falzarano M S, Gualandi F, Ferlini A. Rapid, comprehensive analysis of the dystrophin transcript by a custom micro-fluidic exome array. Hum Mutat. 2012 March; 33(3):572-81.
Bladen C L, Salgado D, Monges S, Foncuberta M E, Kekou K, et al. The TREAT-NMD DMD Global Database: Analysis of More than 7,000 Duchenne Muscular Dystrophy Mutations. Hum Mutat. 2015 April; 36(4): 395-402.
Blake D J, Weir A, Newey S E, Davies K E. Function and genetics of dystrophin and dystrophin-related proteins in muscle. Physiological reviews. 2002; 82(2):291-329.
Brooke M H, Fenichel G M, Griggs R C, Mendell J R, Moxley R, Florence J, et al. Duchenne muscular dystrophy: patterns of clinical progression and effects of supportive therapy. Neurology. 1989; 39(4):475-81.
Bushby K M. Genetic and clinical correlations of Xp21 muscular dystrophy. J Inher Metab Dis. 1992; 15(4):551-564.
Bushby K M, Gardner-Medwin D. The clinical, genetic and dystrophin characteristics of Becker muscular dystrophy. I. Natural history. Journal of neurology. 1993a; 240(2):98-104.
Bushby K M, Gardner-Medwin D, Nicholson L V, et al. The clinical, genetic and dystrophin characteristics of Becker muscular dystrophy. II. Correlation of phenotype with genetic and protein abnormalities. Journal of neurology. February 1993b; 240(2):105-112.
Bushby K, Finkel R, Birnkrant D J, Case L E, Clemens P R, Cripe L, et al. Diagnosis and management of Duchenne muscular dystrophy, part 1: diagnosis, and pharmacological and psychosocial management. The Lancet Neurology. 2010a; 9(1):77-93.
Bushby K, Finkel R, Birnkrant D J, Case L E, Clemens P R, Cripe L, et al. Diagnosis and management of Duchenne muscular dystrophy, part 2: implementation of multidisciplinary care. The Lancet Neurology. 2010b; 9(2):177-89.
Buyse G M, Goemans N, van den Hauwe M, Meier T. Effects of glucocorticoids and idebenone on respiratory function in patients with duchenne muscular dystrophy. Pediatr Pulmonol. 2013 September; 48(9):912-20.
Buyse G M, Voit T, Schara U, Straathof C S, D'Angelo M G, Bernert G, et al; DELOS Study Group. Efficacy of idebenone on respiratory function in patients with Duchenne muscular dystrophy not using glucocorticoids (DELOS): a double-blind randomised placebo-controlled phase 3 trial. Lancet. 2015 May 2; 385(9979):1748-57.
Chen Y W, Nagaraju K, Bakay M, McIntyre O, Rawat R, Shi R, et al. Early onset of inflammation and later involvement of TGFbeta in Duchenne muscular dystrophy. Neurology. 2005; 65(6):826-34.

Ciafaloni E, Fox D J, Pandya S, Westfield C P, Puzhankara S, Romitti P A, et al. Delayed diagnosis in Duchenne muscular dystrophy: data from the Muscular Dystrophy Surveillance, Tracking, and Research Network (MD STARnet). J Pediatr. 2009; 155(3):380-5.

Cirak S, Arechavala-Gomeza V, Guglieri M, Feng L, Torelli S, Anthony K, et al. Exon skipping and dystrophin restoration in patients with Duchenne muscular dystrophy after systemic phosphorodiamidate morpholino oligomer treatment: an open-label, phase 2, dose escalation study. Lancet. 2011; 378(9791):595-605.

Cirak S, Feng L, Anthony K, Arechavala-Gomeza V, Torelli S, Sewry C, et al. Restoration of the dystrophin-associated glycoprotein complex after exon skipping therapy in Duchenne muscular dystrophy. Molecular therapy: the journal of the American Society of Gene Therapy. Mol Ther. 2012; 20(2):462-7.
Eagle M, Baudouin S V, Chandler C, Giddings D R, Bullock R, Bushby K. Survival in Duchenne muscular dystrophy: improvements in life expectancy since 1967 and the impact of home nocturnal ventilation. Neuromuscular disorders. 2002; 12(10):926-9.
Emery A E. The muscular dystrophies. Lancet. 2002; 359(9307):687-95.
Engelhardt J A, Fant P, Guionaud S, Henry S P, Leach M W, Louden C, et al. Scientific and regulatory policy committee points-to-consider paper*: Drug-induced vascular injury associated with nonsmall molecule therapeutics in preclinical development: Part 2. Antisense oligonucleotides. Toxicol Pathol 2015; 43(7):935-44.
Food and Drug Administration (FDA). Measuring Dystrophin in Dystrophinopathy Patients and Interpreting the Data Workshop. March 2015a.
http://www.fda.gov/Drugs/NewsEvents/ucm432429.htm.
Food and Drug Administration (FDA). FDA Draft Guidance for Industry, Duchenne Muscular Dystrophy and Related Dystrophinopathies: Developing Drugs for Treatment, June 2015b. Available from: http://www.fda.gov/downloads/Drugs/GuidanceComplianceRegulatoryInformation/Guidances/U CM450229.pdf
Frazier K S, Sobry C, Derr V, Adams M J, Den Besten C, De Kimpe S, et al. Species-specific inflammatory responses as a primary component for the development of glomerular lesions in mice and monkeys following chronic administration of a second-generation antisense oligonucleotide. Toxicol Pathol. 2014; 42:923-35.
Frazier K S. Antisense oligonucleotide therapies: The promise and the challenges from a toxicologic pathologist's perspective. Toxicol Pathol. 2015; 43:78-89.
Goemans N M, Tulinius M, van den Akker J T, Burm B E, Ekhart P F, et al. Systemic administration of PRO051 in Duchenne's muscular dystrophy. N Engl J Med. 2011; 364(16):1513-22.
Goemans N, van den Hauwe M, Wilson R, van Impe A, Klingels K, Buyse G. Ambulatory capacity and disease progression as measured by the 6-minute-walk-distance in Duchenne muscular dystrophy subjects on daily corticosteroids. Neuromuscular disorders: NMD. 2013; 23(8):618-23.
Goemans N. Drisapersen Efficacy and Safety in Duchenne Muscular Dystrophy: Results of a Phase III, Randomized, Double-Blind, Placebo-Controlled Trial (Study DMD114044). Presented at 2013World Muscle Society (Late Breaking Presentation). Asilomar, Calif.
Goemans N, Voit T, McDonald C, Watson C, Kraus J, et al. Drisapersen treatment for Duchenne muscular dystrophy: results of a 96-week follow-up of an open-label extension study following 2 placebo-controlled trials. 66th Annual Meeting of the American Academy of Neurology (AAN); April 26-May 3, 2014; Philadelphia, Pa., USA
Goldberg L R, Hausmanowa-Petrusewicz I, Fidzianska A, Duggan D J, Steinberg L S, Hoffman E P. A dystrophin missense mutation showing persistence of dystrophin and dystrophin-associated proteins yet a severe phenotype. Annals of neurology. December 1998; 44(6):971-976.
Goyenvalle A, Vulin A, Fougerousse F, Leturcq F, Kaplan J C, Garcia L, Danos O. Rescue of dystrophic muscle through U7 snRNA-mediated exon skipping. Science. 2004 Dec. 3; 306(5702):1796-9.
Gregorevic P, Allen J M, Minami E, Blankenship M J, Haraguchi M, Meuse L, et al. rAAV6-microdystrophin preserves muscle function and extends lifespan in severely dystrophic mice. Nat Med. 2006 July; 12(7):787-9.
Hahn A, Bach J R, Delaubier A, Renardel-Irani A, Guillou C, Rideau Y. Clinical implications of maximal respiratory pressure determinations for individuals with Duchenne muscular dystrophy. Arch Phys Med Rehabil. 1997 January; 78(1):1-6.
Hathout Y, Brody E, Clemens P R, Cripe L, DeLisle R K, Furlong P, et al. Large-scale serum protein biomarker discovery in Duchenne muscular dystrophy. Proceedings of the National Academy of Sciences of the United States of America. 2015; 112(23):7153-8.
The Harriet Lane Handbook, 20th Edition. Mobile Medicine Series, Johns Hopkins Hospital, Branden Engorn, MD and Jamie Flerlage, MD. 2015.
Heemskerk H A, de Winter C L, de Kimpe S J, et al. In vivo comparison of 2′-O-methyl phosphorothioate and morpholino antisense oligonucleotides for Duchenne muscular dystrophy exon skipping. The journal of gene medicine. March 2009; 11(3):257-266.
Henricson E K, Abresch R T, Cnaan A, Hu F, Duong T, Arrieta A, et al. The cooperative international neuromuscular research group Duchenne natural history study: glucocorticoid treatment preserves clinically meaningful functional milestones and reduces rate of disease progression as measured by manual muscle testing and other commonly used clinical trial outcome measures. Muscle & Nerve. 2013a; 48(1):55-67.
Henricson E, Abresch R, Han J, Nicorici A, Keller E G, et al. The 6-Minute Walk Test and Person-Reported Outcomes in Boys with Duchenne Muscular Dystrophy and Typically Developing Controls: Longitudinal Comparisons and Clinically-Meaningful Changes Over One Year. Version 1. PLoS Curr. 2013b Jul. 8; 5: ecurrents.md.9e17658b007eb79fcd6f723089f79e06.
Henry S P, Kim T-W, Kramer-Strickland K, Zanardi T, Fey R A, Levin A A. Toxicologic properties of 2′-O-methoxyethyl chimeric antisense inhibitors in animals and man. In: Antisense Drug Technology—Principles, Strategies, and Applications, 2nd Ed. (Crooke S T, ed.), pp. 327-63, New York: CRC Press, 2008.
Hoffman E P, Brown R H, Jr., Kunkel L M. Dystrophin: The protein product of the duchenne muscular dystrophy locus. Cell. 1987; 51(6):919-28.
Hoffman E P, Fischbeck K H, Brown R H, et al. Characterization of dystrophin in muscle-biopsy specimens from patients with Duchenne's or Becker's muscular dystrophy. The New England journal of medicine. May 26 1988; 318(21):1363-1368.
Hoffman E P, Kunkel L M, Angelini C, et al. Improved diagnosis of Becker muscular dystrophy by dystrophin testing. Neurology. August 1989; 39(8):1011-1017.
Humbertclaude V, Hamroun D, Bezzou K, Berard C, Boespflug-Tanguy O, et al. Motor and respiratory heterogeneity in Duchenne patients: implication for clinical trials. Eur J Paediatr Neurol. 2012 March; 16(2):149-60.
International Conference on Harmonisation: Choice of Control Group and Related Design 2340 and Conduct Issues in Clinical Trials (ICH E-10), Food and Drug Administration, DHHS, 2341 July 2000.
Kaspar R W, Allen H D, Ray W C, Alvarez C E, Kissel J T, Pestronk A, et al. Analysis of dystrophin deletion mutations predicts age of cardiomyopathy onset in becker muscular dystrophy. Circulation Cardiovascular genetics. 2009; 2(6):544-51.
Kawano R, Ishizaki M, Maeda Y, Uchida Y, Kimura E, Uchino M. Transduction of full-length dystrophin to multiple skeletal muscles improves motor performance and life span in utrophin/dystrophin double knockout mice. Mol Ther. 2008 May; 16(5):825-31.
Khirani S, Ramirez A, Aubertin G, Boule M, Chemouny C, et al. Respiratory muscle decline in Duchenne muscular dystrophy. Pediatr Pulmonol. 2014; 49(5):473-81.
Kinali M, Main M, Eliahoo J, Messina S, Knight R K, Lehovsky J, et al. Predictive factors for the development of scoliosis in Duchenne muscular dystrophy. European journal of paediatric neurology:EJPN:official journal of the European Paediatric Neurology Society. 2007; 11(3):160-6.
Kobayashi Y M and Campbell K P. Skeletal Muscle Dystrophin-Glycoprotein Complex and Muscular Dystrophy: Muscle Fundamental Biology and Mechanisms of Disease Volume 2. (J A Hill and E N Olson), pp. 935-942. Academic Press. 2012.
Kohler M, Clarenbach C F, Bahler C, Brack T, Russi E W, Bloch K E. Disability and survival in Duchenne muscular dystrophy. Journal of Neurology, Neurosurgery, and Psychiatry. 2009; 80(3):320-5.
Kole R, Leppert B J. Targeting mRNA Splicing as a Potential Treatment for Duchenne Muscular Dystrophy. Discovery Medicine. 2012; 14(74):59-69.
Lenk U, Oexle K, Voit T, et al. A cysteine 3340 substitution in the dystroglycan-binding domain of dystrophin associated with Duchenne muscular dystrophy, mental retardation and absence of the ERG b-wave. Human molecular genetics. July 1996; 5(7):973-975.
Levin A A, Monteith D K, Leeds J M, Nicklin P L, Geary R S, Butler M, et al. Toxicity of oligodeoxynucleotide therapeutic agents: Antisense Research and Application (Crooke S T), pp. 169-215, Heidelberg: Springer-Verlag, 1998.
Levin A A, Henry S P, Monteith D K, Templin M V. Toxicity of oligodeoxynucleotides: Antisense Drug Technology—Principles, Strategies, and Applications, 1st Ed. (Crooke S T), pp. 201-267, Marcel Dekker, 2001.
Magliano. Psychological and practical difficulties among parents and healthy siblings of children with Duchenne vs. Becker muscular dystrophy: an Italian comparative study. Acta Myol. 2014 December; 33(3):136-43.
Manzur A Y, Kuntzer T, Pike M, Swan A. Glucocorticoid corticosteroids for Duchenne muscular dystrophy. The Cochrane Database of Systematic Reviews. 2009(2):CD003725.
Mayer O H, Finkel R S, Rummey C, Benton M J, Glanzman A M, Flickinger J, Lindstrom B M, Meier T. Characterization of pulmonary function in Duchenne Muscular Dystrophy. Pediatr Pulmonol. 2015 May; 50(5):487-94.
Mayhew A, Cano S, Scott E, Eagle M, Bushby K, et al. Moving towards meaningful measurement: Rasch analysis of the North Star Ambulatory Assessment in Duchenne muscular dystrophy. Dev Med Child Neurol 2011; 53:535-542.
Mazzone E S, Messina S, Vasco G, Main M, Eagle M, et al. Reliability of the North Star Ambulatory Assessment in a multicentric setting. Neuromuscul Disord 2009; 9: 458-461
Mazzone E, Martinelli D, Berardinelli A, Messina S, D'Amico A, et al. North Star Ambulatory Assessment, 6-minute walk test and timed items in ambulant boys with Duchenne muscular dystrophy. Neuromuscul Disord 2010; 20: 712-716.
Mazzone E S, Pane M, Sormani M P, Scalise R, Berardinelli A, Messina S, et al. 24 month longitudinal data in ambulant boys with Duchenne muscular dystrophy. PloS one. 2013;8(1):e52512.
McDonald C M, Abresch R T, Carter G T, Fowler W M, Jr., Johnson E R, Kilmer D D. Profiles of neuromuscular diseases. Becker's muscular dystrophy. American Journal of Physical Medicine & R/Association of Academic Physiatrists. 1995; 74(5 Suppl):593-103.
McDonald C M, Henricson E K, Han J J, Abresch R T, Nicorici A, Elfring G L, et al. The 6-minute walk test as a new outcome measure in Duchenne muscular dystrophy. Muscle Nerve. 2010a April; 41(4):500-10.
McDonald C M, Henricson E K, Han J J, Abresch R T, Nicorici A, Atkinson L, et al. The 6-minute walk test in Duchenne/Becker muscular dystrophy: longitudinal observations. Muscle & Nerve. 2010b; 42(6):966-74.
McDonald C M, Henricson E K, Abresch R T, Han J J, Escolar D M, Florence J M, et al. The cooperative international neuromuscular research group Duchenne natural history study—a longitudinal investigation in the era of glucocorticoid therapy: design of protocol and the methods used. Muscle & Nerve. 2013b; 48(1):32-54.
McGowan M P, Tardif J C, Ceska R, Burgess L J, Soran H, et al. Randomized, placebo-controlled trial of mipomersen in patients with severe hypercholesterolemia receiving maximally tolerated lipid-lowering therapy. PLoS One. 2012; 7(11):e49006.
McMillan H J, Gregas M, Darras B T, and Kang P B. Serum Transaminase Levels in Boys With Duchenne and Becker Muscular Dystrophy. Pediatr. 2011; 127(1):132-6.
MedCalc: Online Clinical Calculators. (n.d.). Retrieved Oct. 25, 2015
Mendell J R, Shilling C, Leslie N D, Flanigan K M, al-Dahhak R, Gastier-Foster J, et al. Evidence-based path to newborn screening for Duchenne muscular dystrophy. Annals of Neurology. 2012; 71(3):304-13.
Mendell J R, Rodino-Klapac L R, Sahenk Z, Roush K, Bird L, et al. Eteplirsen for the treatment of Duchenne muscular dystrophy. Ann Neurol. 2013 November; 74(5):637-47.
Mendell J R, Goemans N, Lowes L P, Alfano L N, Berry K, Shao J, et al. Longitudinal effect of eteplirsen vs. historical control on ambulation in DMD. Annals of Neurology. 2016 February; 79(2):257-71.
Miller M R, Hankinson J, Brusasco V, Burgos F, Casaburi R, Coates A, et al. Standardisation of spirometry. 2005; 26(2):319-38.
Moat S J, Bradley D M, Salmon R, Clarke A, Hartley L. Newborn bloodspot screening for Duchenne muscular dystrophy: 21 years experience in Wales (UK). European Journal of Human Genetics. 2013; 21(10):1049-53.
Monteith D K, Homer M J, Gillett N A, Butler M, Geary R, Burckin T, et al. Evaluation of the renal effects of an antisense phosphorothioate oligodeoxynucleotide in monkeys. Toxicol Pathol. 1999; 27(3):307-17.
Moxley R T 3rd, Pandya S, Ciafaloni E, Fox D J, Campbell K. Change in natural history of Duchenne muscular dystrophy with long-term corticosteroid treatment: implications for management. J Child Neurol. 2010 September; 25(9):1116-29.
Nguyen, T. M., Ginjaar, H. B., Van Ommen, G. J. B., and Morris, G. E. Monoclonal antibodies for dystrophin analysis: epitope mapping and improved binding to SDS-treated muscle sections. Biochem. J. 1992; 288:663-668.
Nicholson L V, Johnson M A, Bushby K M, et al. Integrated study of 100 patients with Xp21 linked muscular dystrophy using clinical, genetic, immunochemical, and histopathological data. Part 1. Trends across the clinical groups. Journal of medical genetics. 1993 September; 30(9):728-736. Pane M, Mazzone E S, Sormani M P, Messina S, Vita G L, Fanelli L, et al. 6 Minute walk test in Duchenne M D patients with different mutations: 12 month changes. PloS one. 2014a; 9(1):e83400.
Pane M, Mazzone E S, Sivo S, Sormani M P, Messina S, A D A, et al. Long term natural history data in ambulant boys with Duchenne muscular dystrophy: 36-month changes. PloS one. 2014b; 9(10):e108205.
Polgar G, Promadhat V. Pulmonary function testing in children: techniques and standards. Philadelphia: W B Saunders C, 1971.
Pradhan S, Ghosh D, Srivastava N K, Kumar A, Mittal B, Pandey C M, et al. Prednisolone in Duchenne muscular dystrophy with imminent loss of ambulation. Journal of Neurology. 2006; 253(10):1309-16.
Quanjer P H, Stanojevic S, Cole T J, Baur X, Hall G L, Culver B H, Enright P L, Hankinson J L, Ip M S, Zheng J, Stocks J. Initiative EGLF. Multi-ethnic reference values for spirometry for the 3-95-yr age range: the global lung function 2012 equations. Eur Resp J 2012; 40:1324-1343.
Reay D P, Bilbao R, Koppanati B M, Cai L, O'Day T L, Jiang Z, et al. Full-length dystrophin gene transfer to the mdx mouse in utero. Gene Ther. 2008 April; 15(7):531-6.
Ricotti V, Ridout D A, Scott E, Quinlivan R, Robb S A, Manzur A Y, et al. Long-term benefits and adverse effects of intermittent versus daily glucocorticoids in boys with Duchenne muscular dystrophy. Journal of neurology, neurosurgery, and psychiatry. 2013; 84(6):698-705.
Ricotti V, Ridout D A, Pane M, Main M, Mayhew A, Mercuri E, et al. The NorthStar Ambulatory Assessment in Duchenne muscular dystrophy: considerations for the design of clinical trials. Journal of Neurology, Neurosurgery, and Psychiatry. 2015; 0:1-7.
Sanger F, Coulson A R. A rapid method for determining sequences in DNA by primed synthesis with DNA polymerase. J Mol Biol 1975; 94 (3): 441-8.
Sasinowski F J, Panico E B, Valentine J E. Quantum of Effectiveness Evidence in FDA's Approval of Orphan Drugs: Update, July 2010 to June 2014. Therapeutic Innovation & Regulatory Science 2015 April.
Schram G, Fournier A, Leduc H, Dandah N, Therien J, Vanasse M, et al. All-cause mortality and cardiovascular outcomes with prophylactic steroid therapy in Duchenne muscular dystrophy. Journal of the American College of Cardiology. 2013; 61(9):948-54.
Schwartz G J and Gauthier B. A simple estimate of glomerular filtration rate in adolescent boys. J Pediatr. 1985 March; 106(3):522-6.
Scott E, Eagle M, Mayhew A, Freeman J, Main M, Sheehan J, et al. Development of a functional assessment scale for ambulatory boys with Duchenne muscular dystrophy. Physiother Res Int. 2012; 17(2):101-9.
Sharp P S, Bye-a-Jee H, Wells D J. Physiological characterization of muscle strength with variable levels of dystrophin restoration in mdx mice following local antisense therapy. Mol Ther 2011; 19(1):165-171.
Spurney C, Shimizu R, Morgenroth L P, Kolski H, Gordish-Dressman H, Clemens P R; CINRG Investigators. Cooperative International Neuromuscular Research Group Duchenne Natural History Study demonstrates insufficient diagnosis and treatment of cardiomyopathy in Duchenne muscular dystrophy. Muscle Nerve. 2014 August; 50(2):250-6.
Takeuchi F, Yonemoto N, Nakamura H, Shimizu R, Komaki H, Mori-Yoshimura M, et al. Prednisolone improves walking in Japanese Duchenne muscular dystrophy patients. Journal of neurology. 2013; 260(12):3023-9.
Taylor L E, Kaminoh Y J, Rodesch C K, Flanigan K M. Quantification of dystrophin immunofluorescence in dystrophinopathy muscle specimens. Neuropathology and applied neurobiology. October 2012; 38(6):591-601.
Tennyson C N, Klamut H J, Worton R G. The human dystrophin gene requires 16 hours to be transcribed and is cotranscriptionally spliced. Nat Genet. 1995; 9(2):184-90.
Thomas T O, Thomas M M, Burnette W B, Markham L W. Correlation of heart rate and cardiac dysfunction in Duchenne muscular dystrophy. Pediatr Cardiol. 2012; 33(7):1175-9.
Uzark K, King E, Cripe L, Spicer R, Sage J, Kinnett K, et al. Health-related quality of life in children and adolescents with Duchenne muscular dystrophy. Pediatrics. 2012; 130(6):e1559-66.
van den Bergen J C, Wokke B H, Janson A A, et al. Dystrophin levels and clinical severity in Becker muscular dystrophy patients. Journal of neurology, neurosurgery, and psychiatry. July 2014; 85(7):747-753.
Van Essen A J, Verheij J B G M, Reefhuis J, Fidler V, Begeer J H, et al. The natural history of Duchenne muscular dystrophy: analysis of data from a Dutch survey and review of age-related events. Online Leyden Muscular Dystrophy pages. 2014. [Accessed 15 Nov. 2015] Available: http://www.dmd.nl/.
van Putten M, Hulsker M, Nadarajah V D, van Heiningen S H, van Huizen E, van Iterson M, et al. The effects of low levels of dystrophin on mouse muscle function and pathology. PLoS One. 2012; 7(2): e31937.
van Ruiten H J, Straub V, Bushby K, Guglieri M. Improving recognition of Duchenne muscular dystrophy: a retrospective case note review. Archives of Disease in Childhood. 2014; 99(12): 1074-7.
Verhaart I E, Heemskerk H, Karnaoukh T G, Kolfschoten I G, Vroon A, van Ommen G J, van Deutekom J C, Aartsma-Rus A. Prednisolone treatment does not interfere with 2′-O-methyl phosphorothioate antisense-mediated exon skipping in Duchenne muscular dystrophy. Hum Gene Ther. 2012 March; 23(3):262-73.
Viollet L, Gailey S, Thornton D J, Friedman N R, Flanigan K, et al. Utility of Cystatin C to monitor renal function in Duchenne muscular dystrophy. Muscle Nerve. 2009; 40(3):438-42.
Voit T, Topaloglu H, Straub V, et al. Safety and efficacy of drisapersen for the treatment of Duchenne muscular dystrophy (DEMAND II): an exploratory, randomised, placebo-controlled phase 2 study. Lancet Neurol 2014; 13:987-996.
Wei X, Dai Y, Yu P, Qu N, Lan Z, et al. Targeted next-generation sequencing as a comprehensive test for patients with and female carriers of DMD/BMD: a multi-population diagnostic study. Eur J Hum Genet. 2014 January; 22(1): 110-118.
Welch E M, Barton E R, Zhuo J, Tomizawa Y, Friesen W J, Trifillis P, et al. PTC124 targets genetic disorders caused by nonsense mutations. Nature. 2007 May; 447(7140):87-91.
Wilson S H, Cooke N T, Edwards R H, Spiro S G. Predicted normal values for maximal respiratory pressures in Caucasian adults and children. Thorax. 1984 July; 39(7):535-8.
Wu B, Moulton H M, Iversen P L, Jiang J, Li J, Li J, et al. Effective rescue of dystrophin improves cardiac function in dystrophin-deficient mice by a modified morpholino oligomer. Proc Natl Acad Sci USA. 2008 September; 105(39):14814-9.
Wu B, Xiao B, Cloer C, Shaban M, Sali A, Lu P, et al. One-year treatment of morpholino antisense oligomer improves skeletal and cardiac muscle functions in dystrophic mdx mice. Mol Ther 2011; 19(3):576-583.
Wu B, Lu P, Cloer C, Shaban M, Grewal S, Milazi S, et al. Long-Term Rescue of Dystrophin Expression and Improvement in Muscle Pathology and Function in Dystrophic mdx Mice by Peptide-Conjugated Morpholino. The American Journal of Pathology. 2012; 181(2):392-400.
Yokota T, Lu Q L, Partridge T, Kobayashi M, Nakamura A, Takeda S, Hoffman E. Efficacy of systemic morpholino exon-skipping in Duchenne dystrophy dogs. Ann Neurol. 2009 June; 65(6):667-76.
Yue Y, Liu M, Duan D. C-terminal-truncated microdystrophin recruits dystrobrevin and syntrophin to the dystrophin-associated glycoprotein complex and reduces muscular dystrophy in symptomatic utrophin/dystrophin double-knockout mice. Mol Ther. 2006 July; 14(1):79-87.
Zatz M, Rappaport D, Vainzof M et al. Serum creatine-kinase (CK) and pyruvate-kinase (PK) activities in Duchenne (DMD) as compared with Becker (BMD) muscular dystrophy. J Neur Sci. 1991; 102(2):190-6.
Zaharieva I T, Calissano M, Scoto M, Preston M, Cirak S, Feng L, et al. Dystromirs as serum biomarkers for monitoring the disease severity in Duchenne muscular Dystrophy. PloS one. 2013; 8(11):e80263.

### SEQUENCE LISTING

In the following Table 3, any “T” that is shown, or all, can be replaced with a “U” and any “U” that is shown, or all, can be replaced by “T”.

In the following Table 3, “X” is “U” or “T” and “C” is 5-methylcytosine.

TABLE 3

| SEQ ID | | |
|---|---|---|
| NO | SEQUENCE | NUCLEOTIDE SEQUENCE (5′-3′) |
| 1 | SRP-4053 (+36+60) | GTTGCCTCCGGTTCTGAAGGTGTTC |
| 2 | PRO053 (+36+60) | GUUGCCUCCGGUUCUGAAGGUGUUC |
| 3 | WO 2004/083432 (SIN: 29) | CUGUUGCCUCCGGUUCUG |
| 4 | WO 2012/029986 (SIN: 11) | CCTCCGGTTCTGAAGGTGTTCTTGT |
| 5 | WO 2012/029986 (SIN:35) | CCTCCGGTTCTGAAGGTGTTC |
| 6 | U.S. Pat. No. 8,084,601 | CXGXXGCCXCCGGXXCXGAAGGXGXXCXXG |
| | (SIN: 10) | |
| 7 | U.S. Pat. No. 8,084,601 | CAACXGXXGCCXCCGGXXCXGAAGGXGXXC |
| | (SIN: 11) | |
| 8 | U.S. Pat. No. 8,084,601 | XXGCCXCCGGXXCXGAAGGXGXXCXXGXAC |
| | (SIN: 12) | |
| 9 | WO 2012/109296 (SIN: 116) | CAACTGTTGCCTCCGGTTCTGAAG |
| 10 | M23D (AVI-4225) | GGCCAAACCTCGGCTTACCTGAAAT |
| 11 | (RXR)₄ | RXRRXRRXRRXR |
| 12 | (RFF)₃R | RFFRFFRFFR |
| 13 | (RXR)₄XB | RXRRXRRXRRXRXB |
| 14 | (RFF)₃RXB | RFFRFFRFFRXB |
| 15 | (RFF)₃RG | RFFRFFRFFR |
| 16 | R₅G | RRRRRG |
| 17 | R₅ | RRRRR |
| 18 | R₆G | RRRRRRG |
| 19 | R₆ | RRRRRR |
| 20 | Viltolarsen | CCTCCGGTTCTGAAGGTGTTC |

1. A method for treating Duchenne muscular dystrophy (DMD) in a patient in need thereof having a mutation of the DMD gene that is amenable to exon 53 skipping, comprising
administering to the patient an effective amount of golodirsen and an effective amount of a non-steroidal anti-inflammatory compound,
thereby treating the patient with DMD.

2. The method of claim 1, wherein the non-steroidal anti-inflammatory compound is an NF-kB inhibitor.

3. The method of claim 2, wherein the NF-kB inhibitor is selected from edasalonexent or CAT-1041 or pharmaceutically acceptable salts thereof.

4. The method of claim 1, wherein golodirsen is administered at a dose of 30 mg/kg weekly.

5. The method of claim 3, wherein edasalonexent is administered at a dose of 67 mg/kg/day.

6. The method of claim 3, wherein edasalonexent is administered at a dose of 100 mg/kg/day.

7. The method of claim 1, wherein the non-steroidal anti-inflammatory compound is administered for at least 12 weeks prior to initially administering golodirsen.

8. The method of claim 1, wherein golodirsen and the non-steroidal anti-inflammatory compound are administered simultaneously or sequentially.

9. The method of claim 8, wherein golodirsen is administered prior to the administration of the non-steroidal anti-inflammatory compound.

10. The method of claim 8, wherein the non-steroidal anti-inflammatory compound is administered prior to the administration of golodirsen.

11. The method of any of the preceding claims, wherein treatment results in reduced muscle inflammation in the patient relative to administration of golodirsen or the non-steroidal anti-inflammatory compound alone.

12. The method of any of the preceding claims, wherein treatment results in reduced muscle fibrosis in the patient relative to either golodirsen or the non-steroidal anti-inflammatory compound alone.

13. The method of any of the preceding claims, wherein treatment results in increased dystrophin in the patient relative to administration of golodirsen or the non-steroidal anti-inflammatory compound alone.

14. A method for inducing or increasing dystrophin protein production in a patient with Duchenne muscular dystrophy (DMD) in need thereof who has a mutation of the DMD gene that is amenable to exon 53 skipping, comprising administering to the patient an effective amount of golodirsen; and an effective amount of a non-steroidal anti-inflammatory compound, thereby inducing or increasing dystrophin protein production in the patient.

15. The method of claim 14, wherein the non-steroidal anti-inflammatory compound is an NF-kB inhibitor.

16. The method of claim 15, wherein the NF-kB inhibitor is selected from edasalonexent or CAT-1041 or pharmaceutically acceptable salts thereof.

17. The method of any of the preceding claims, wherein golodirsen and the non-steroidal anti-inflammatory compound are administered simultaneously.

18. The method of any of the preceding claims, wherein golodirsen and the non-steroidal anti-inflammatory compound are administered sequentially.

19. Use of golodirsen, and an optional pharmaceutically acceptable carrier, in the manufacture of a medicament for treating or delaying progression of DMD in a patient, wherein the medicament comprises golodirsen and an optional pharmaceutically acceptable carrier, and wherein the treatment comprises administration of the medicament in combination with edasalonexent, and an optional pharmaceutically acceptable carrier.

20. Golodirsen, and an optional pharmaceutically acceptable carrier, for use in treating or delaying progression of DMD in a patient, wherein the treatment comprises administration of golodirsen in combination with a second composition, wherein the second composition comprises edasalonexent and an optional pharmaceutically acceptable carrier.

21. A kit comprising a container comprising edasalonexent, and an optional pharmaceutically acceptable carrier, and a package insert comprising instructions for administration of edasalonexent in combination with a golodirsen, an optional pharmaceutically acceptable carrier for treating or delaying progression of DMD in a patient.
